SEQUENCE LISTING (763 sequences)

SEQ ID NO: 1 - DNA, 351 bp, synthetic construct
  misc_feature 1..351: note = Synthetic
  source 1..351: mol_type = other DNA
caggtccagctggtgcagtctgggggaggcttggtacagcctggggggtccctgagactctcctgtgcagcctctggatttactctaagtagttacgacatgcactgggtccgccaatctacaggaaaaggtctggagtgggtctcagctattggttctaccggtgacacatactatccaggctccgtgaagggccgattcaccatcaccagagaaaaagccaagaactccgtgtatcttcaaatgaacagcctgagagccggggacacggctgtgtattactgtgtaagagaggggtgggaggtaccctttgactactggggccagggaaccctggtcactgtctcctca

SEQ ID NO: 2 - protein, 117 aa, synthetic construct
  REGION 1..117: note = Synthetic
QVQLVQSGGGLVQPGGSLRLSCAASGFTLSSYDMHWVRQSTGKGLEWVSAIGSTGDTYYPGSVKGRFTITREKAKNSVYLQMNSLRAGDTAVYYCVREGWEVPFDYWGQGTLVTVSS

SEQ ID NO: 3 - DNA, 24 bp, synthetic construct
  misc_feature 1..24: note = Synthetic
  source 1..24: mol_type = other DNA
ggatttactctaagtagttacgac

SEQ ID NO: 4 - protein, 8 aa, synthetic construct
  REGION 1..8: note = Synthetic
GFTLSSYD

SEQ ID NO: 5 - DNA, 21 bp, synthetic construct
  misc_feature 1..21: note = Synthetic
  source 1..21: mol_type = other DNA
attggttctaccggtgacaca

SEQ ID NO: 6 - protein, 7 aa, synthetic construct
  REGION 1..7: note = Synthetic
IGSTGDT

SEQ ID NO: 7 - DNA, 33 bp, synthetic construct
  misc_feature 1..33: note = Synthetic
  source 1..33: mol_type = other DNA
gtaagagaggggtgggaggtaccctttgactac

SEQ ID NO: 8 - protein, 11 aa, synthetic construct
  REGION 1..11: note = Synthetic
VREGWEVPFDY

SEQ ID NO: 9 - DNA, 327 bp, synthetic construct
  misc_feature 1..327: note = Synthetic
  source 1..327: mol_type = other DNA
gacatccagatgacccagtctccagccaccctgtctgtgtctccaggggaaagagccgccctctcctgcagggccagtcagagtgttagcagcaacttagcctggtaccaccagaaacctggccaggctcccaggctcctcatctatggtgcatccaccagggccactggtatcccagccaggttcagtggcattgggtctgggacagagttcactctcattatcagcagcctgcagtctgaagattttgcattttatttctgtcagcagtataataactggcctccattcactttcggccctgggaccaaggtggagatcaaacga

SEQ ID NO: 10 - protein, 109 aa, synthetic construct
  REGION 1..109: note = Synthetic
DIQMTQSPATLSVSPGERAALSCRASQSVSSNLAWYHQKPGQAPRLLIYGASTRATGIPARFSGIGSGTEFTLIISSLQSEDFAFYFCQQYNNWPPFTFGPGTKVEIKR

SEQ ID NO: 11 - DNA, 18 bp, synthetic construct
  misc_feature 1..18: note = Synthetic
  source 1..18: mol_type = other DNA
cagagtgttagcagcaac

SEQ ID NO: 12 - protein, 6 aa, synthetic construct
  REGION 1..6: note = Synthetic
QSVSSN

SEQ ID NO: 13-14 - skipped (2 entries)

SEQ ID NO: 15 - DNA, 30 bp, synthetic construct
  misc_feature 1..30: note = Synthetic
  source 1..30: mol_type = other DNA
cagcagtataataactggcctccattcact

SEQ ID NO: 16 - protein, 10 aa, synthetic construct
  REGION 1..10: note = Synthetic
QQYNNWPPFT

SEQ ID NO: 17 - DNA, 351 bp, synthetic construct
  misc_feature 1..351: note = Synthetic
  source 1..351: mol_type = other DNA
gaggtgcagctggtggagtctgggggaggcttggtacagcctggggggtccctgagactctcctgtgcagcctctggatttactctaagtagttacgacatgcactgggtccgccaatctacaggaaaaggtctggagtgggtctcagctattggttctaccggtgacacatactatccaggctccgtgaagggccgattcaccatcaccagagaaaaagccaagaactccgtgtatcttcaaatgaacagcctgagagccggggacacggctgtgtattactgtgtaagagaggggtgggaggtaccctttgactactggggccagggaaccctggtcaccgtctcctca

SEQ ID NO: 18 - protein, 117 aa, synthetic construct
  REGION 1..117: note = Synthetic
EVQLVESGGGLVQPGGSLRLSCAASGFTLSSYDMHWVRQSTGKGLEWVSAIGSTGDTYYPGSVKGRFTITREKAKNSVYLQMNSLRAGDTAVYYCVREGWEVPFDYWGQGTLVTVSS

SEQ ID NO: 19 - DNA, 324 bp, synthetic construct
  misc_feature 1..324: note = Synthetic
  source 1..324: mol_type = other DNA
gaaatagtgatgacgcagtctccagccaccctgtctgtgtctccaggggaaagagccgccctctcctgcagggccagtcagagtgttagcagcaacttagcctggtaccaccagaaacctggccaggctcccaggctcctcatctatggtgcatccaccagggccactggtatcccagccaggttcagtggcattgggtctgggacagagttcactctcattatcagcagcctgcagtctgaagattttgcattttatttctgtcagcagtataataactggcctccattcactttcggccctgggaccaaagtggatatcaaa

SEQ ID NO: 20 - protein, 108 aa, synthetic construct
  REGION 1..108: note = Synthetic
EIVMTQSPATLSVSPGERAALSCRASQSVSSNLAWYHQKPGQAPRLLIYGASTRATGIPARFSGIGSGTEFTLIISSLQSEDFAFYFCQQYNNWPPFTFGPGTKVDIK

SEQ ID NO: 21 - DNA, 351 bp, synthetic construct
  misc_feature 1..351: note = Synthetic
  source 1..351: mol_type = other DNA
gaggtgcagctggtggagtctgggggaggcttggtacagcctggggggtccctgagactctcctgtgcagcctctggatttactctaagtagttacgacatgcactgggtccgccaagctacaggaaaaggtctggagtgggtctcagctattggttctaccggtgacacatactatccaggctccgtgaagggccgattcaccatctccagagaaaatgccaagaactccttgtatcttcaaatgaacagcctgagagccggggacacggctgtgtattactgtgtaagagaggggtgggaggtaccctttgactactggggccagggaaccctggtcaccgtctcctca

SEQ ID NO: 22 - protein, 117 aa, synthetic construct
  REGION 1..117: note = Synthetic
EVQLVESGGGLVQPGGSLRLSCAASGFTLSSYDMHWVRQATGKGLEWVSAIGSTGDTYYPGSVKGRFTISRENAKNSLYLQMNSLRAGDTAVYYCVREGWEVPFDYWGQGTLVTVSS

SEQ ID NO: 23 - DNA, 324 bp, synthetic construct
  misc_feature 1..324: note = Synthetic
  source 1..324: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctccattcactttcggccctgggaccaaagtggatatcaaa

SEQ ID NO: 24 - protein, 108 aa, synthetic construct
  REGION 1..108: note = Synthetic
EIVMTQSPATLSVSPGERATLSCRASQSVSSNLAWYQQKPGQAPRLLIYGASTRATGIPARFSGSGSGTEFTLTISSLQSEDFAVYYCQQYNNWPPFTFGPGTKVDIK

SEQ ID NO: 25 - DNA, 342 bp, synthetic construct
  misc_feature 1..342: note = Synthetic
  source 1..342: mol_type = other DNA
caggtgcagctggtgcagtctgggggaggcgtggtccagcctgggaggtccctgagactctcctgtgcagcgtctggattcaccttcagtagctatggcatgcactgggtccgccaggctccaggcaaggggctggagtgggtggcgtttataggatttgatggaagtaatatacattatggagactccgtgaggggccgaatcatcatatccagagacaattccgagaacacgttgtatctggaaatgaacagcctgagagccgaggacacggcaatgtactattgtgcgagagagaagggtttagactggggccagggaaccacggtcaccgtctcctca

SEQ ID NO: 26 - protein, 114 aa, synthetic construct
  REGION 1..114: note = Synthetic
QVQLVQSGGGVVQPGRSLRLSCAASGFTFSSYGMHWVRQAPGKGLEWVAFIGFDGSNIHYGDSVRGRIIISRDNSENTLYLEMNSLRAEDTAMYYCAREKGLDWGQGTTVTVSS

SEQ ID NO: 27 - DNA, 24 bp, synthetic construct
  misc_feature 1..24: note = Synthetic
  source 1..24: mol_type = other DNA
ggattcaccttcagtagctatggc

SEQ ID NO: 28 - protein, 8 aa, synthetic construct
  REGION 1..8: note = Synthetic
GFTFSSYG

SEQ ID NO: 29 - DNA, 24 bp, synthetic construct
  misc_feature 1..24: note = Synthetic
  source 1..24: mol_type = other DNA
ataggatttgatggaagtaatata

SEQ ID NO: 30 - protein, 8 aa, synthetic construct
  REGION 1..8: note = Synthetic
IGFDGSNI

SEQ ID NO: 31 - DNA, 21 bp, synthetic construct
  misc_feature 1..21: note = Synthetic
  source 1..21: mol_type = other DNA
gcgagagagaagggtttagac

SEQ ID NO: 32 - protein, 7 aa, synthetic construct
  REGION 1..7: note = Synthetic
AREKGLD

SEQ ID NO: 33 - DNA, 321 bp, synthetic construct
  misc_feature 1..321: note = Synthetic
  source 1..321: mol_type = other DNA
gccatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattacacttttggccaggggaccaaggtggaaatcaaacga

SEQ ID NO: 34 - protein, 107 aa, synthetic construct
  REGION 1..107: note = Synthetic
AIQMTQSPSTLSASVGDRVTITCRASQSISSWLAWYQQKPGKAPKLLIYKASSLESGVPSRFSGSGSGTEFTLTISSLQPDDFATYYCQQYNSYYTFGQGTKVEIKR

SEQ ID NO: 35 - DNA, 18 bp, synthetic construct
  misc_feature 1..18: note = Synthetic
  source 1..18: mol_type = other DNA
cagagtattagtagctgg

SEQ ID NO: 36 - protein, 6 aa, synthetic construct
  REGION 1..6: note = Synthetic
QSISSW

SEQ ID NO: 37-38 - skipped (2 entries)

SEQ ID NO: 39 - DNA, 24 bp, synthetic construct
  misc_feature 1..24: note = Synthetic
  source 1..24: mol_type = other DNA
caacagtataatagttattacact

SEQ ID NO: 40 - protein, 8 aa, synthetic construct
  REGION 1..8: note = Synthetic
QQYNSYYT

SEQ ID NO: 41 - DNA, 342 bp, synthetic construct
  misc_feature 1..342: note = Synthetic
  source 1..342: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcgtttataggatttgatggaagtaatatacattatggagactccgtgaggggccgaatcatcatatccagagacaattccgagaacacgttgtatctggaaatgaacagcctgagagccgaggacacggcaatgtactattgtgcgagagagaagggtttagactggggccagggaaccctggtcaccgtctcctca

SEQ ID NO: 42 - protein, 114 aa, synthetic construct
  REGION 1..114: note = Synthetic
QVQLVESGGGVVQPGRSLRLSCAASGFTFSSYGMHWVRQAPGKGLEWVAFIGFDGSNIHYGDSVRGRIIISRDNSENTLYLEMNSLRAEDTAMYYCAREKGLDWGQGTLVTVSS

SEQ ID NO: 43 - DNA, 318 bp, synthetic construct
  misc_feature 1..318: note = Synthetic
  source 1..318: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattacacttttggccaggggaccaagctggagatcaaa

SEQ ID NO: 44 - protein, 106 aa, synthetic construct
  REGION 1..106: note = Synthetic
DIQMTQSPSTLSASVGDRVTITCRASQSISSWLAWYQQKPGKAPKLLIYKASSLESGVPSRFSGSGSGTEFTLTISSLQPDDFATYYCQQYNSYYTFGQGTKLEIK

SEQ ID NO: 45 - DNA, 342 bp, synthetic construct
  misc_feature 1..342: note = Synthetic
  source 1..342: mol_type = other DNA
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttataggatttgatggaagtaatatatactatgcagactccgtgaagggccgattcaccatctccagagacaattccaagaacacgctgtatctgcaaatgaacagcctgagagctgaggacacggctgtgtattactgtgcgagagagaagggtttagactggggccagggaaccctggtcaccgtctcctca

SEQ ID NO: 46 - protein, 114 aa, synthetic construct
  REGION 1..114: note = Synthetic
QVQLVESGGGVVQPGRSLRLSCAASGFTFSSYGMHWVRQAPGKGLEWVAVIGFDGSNIYYADSVKGRFTISRDNSKNTLYLQMNSLRAEDTAVYYCAREKGLDWGQGTLVTVSS

SEQ ID NO: 47 - DNA, 319 bp, synthetic construct
  misc_feature 1..319: note = Synthetic
  source 1..319: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattacacttttggccaggggaccaagctggagatcaaac

SEQ ID NO: 48 - protein, 106 aa, synthetic construct; identical to SEQ ID NO: 44
  REGION 1..106: note = Synthetic

SEQ ID NO: 49 - DNA, 342 bp, synthetic construct
  misc_feature 1..342: note = Synthetic
  source 1..342: mol_type = other DNA
caggtgcagctgcaggagtctgggggaggcgtggtccagcctgggaggtccctgagactctcctgtgcagcgtctggattcaccttcagtagctatggcatgcactgggtccgccaggctccaggcaaggggctggagtgggtggcgtttataggatttgatggaagtaatatatattatggagactccgtgaggggccgaatcatcatatccagagacaattccgagaacacgttgtatctggaaatgaacagcctgagagccgaggacacggcagtgtattattgtgcgagagagaagggtttagactggggccagggaaccctggtcactgtctcctca

SEQ ID NO: 50 - protein, 114 aa, synthetic construct
  REGION 1..114: note = Synthetic
QVQLQESGGGVVQPGRSLRLSCAASGFTFSSYGMHWVRQAPGKGLEWVAFIGFDGSNIYYGDSVRGRIIISRDNSENTLYLEMNSLRAEDTAVYYCAREKGLDWGQGTLVTVSS

SEQ ID NO: 51 - DNA, 24 bp, synthetic construct
  misc_feature 1..24: note = Synthetic
  source 1..24: mol_type = other DNA
ggattcaccttcagtagctatggc

SEQ ID NO: 52 - protein, 8 aa, synthetic construct
  REGION 1..8: note = Synthetic
GFTFSSYG

SEQ ID NO: 53 - DNA, 24 bp, synthetic construct
  misc_feature 1..24: note = Synthetic
  source 1..24: mol_type = other DNA
ataggatttgatggaagtaatata

SEQ ID NO: 54 - protein, 8 aa, synthetic construct
  REGION 1..8: note = Synthetic
IGFDGSNI

SEQ ID NO: 55 - DNA, 21 bp, synthetic construct
  misc_feature 1..21: note = Synthetic
  source 1..21: mol_type = other DNA
gcgagagagaagggtttagac

SEQ ID NO: 56 - protein, 7 aa, synthetic construct
  REGION 1..7: note = Synthetic
AREKGLD

SEQ ID NO: 57 - DNA, 342 bp, synthetic construct
  misc_feature 1..342: note = Synthetic
  source 1..342: mol_type = other DNA
gccatccagatgacccagtctccagactccctggctgtgtctctgggcgagagggccaccatcaactgcaagtccagccagagtgtttttcacacctccaacaataagaactacttagtttggtatcagcagaaaccaggacagcctcctaagttgctcctttactgggcctctacccgggaatccggggtccctgaccgattcagtggcagcgggtctgggacagatttcactctcaccatcagcagcctgcaggctgaagatgtggcaaattattactgtcaccaatattacagtattccgtggacgttcggccaagggaccaaggtggagatcaaacga

SEQ ID NO: 58 - protein, 114 aa, synthetic construct
  REGION 1..114: note = Synthetic
AIQMTQSPDSLAVSLGERATINCKSSQSVFHTSNNKNYLVWYQQKPGQPPKLLLYWASTRESGVPDRFSGSGSGTDFTLTISSLQAEDVANYYCHQYYSIPWTFGQGTKVEIKR

SEQ ID NO: 59 - DNA, 36 bp, synthetic construct
  misc_feature 1..36: note = Synthetic
  source 1..36: mol_type = other DNA
cagagtgtttttcacacctccaacaataagaactac

SEQ ID NO: 60 - protein, 12 aa, synthetic construct
  REGION 1..12: note = Synthetic
QSVFHTSNNKNY

SEQ ID NO: 61-62 - skipped (2 entries)

SEQ ID NO: 63 - DNA, 27 bp, synthetic construct
  misc_feature 1..27: note = Synthetic
  source 1..27: mol_type = other DNA
caccaatattacagtattccgtggacg

SEQ ID NO: 64 - protein, 9 aa, synthetic construct
  REGION 1..9: note = Synthetic
HQYYSIPWT

SEQ ID NO: 65 - DNA, 342 bp, synthetic construct
  misc_feature 1..342: note = Synthetic
  source 1..342: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcgtttataggatttgatggaagtaatatatattatggagactccgtgaggggccgaatcatcatatccagagacaattccgagaacacgttgtatctggaaatgaacagcctgagagccgaggacacggcagtgtattattgtgcgagagagaagggtttagactggggccagggaaccctggtcaccgtctcctca

SEQ ID NO: 66 - protein, 114 aa, synthetic construct
  REGION 1..114: note = Synthetic
QVQLVESGGGVVQPGRSLRLSCAASGFTFSSYGMHWVRQAPGKGLEWVAFIGFDGSNIYYGDSVRGRIIISRDNSENTLYLEMNSLRAEDTAVYYCAREKGLDWGQGTLVTVSS

SEQ ID NO: 67 - DNA, 339 bp, synthetic construct
  misc_feature 1..339: note = Synthetic
  source 1..339: mol_type = other DNA
gacatcgtgatgacccagtctccagactccctggctgtgtctctgggcgagagggccaccatcaactgcaagtccagccagagtgtttttcacacctccaacaataagaactacttagtttggtatcagcagaaaccaggacagcctcctaagttgctcctttactgggcctctacccgggaatccggggtccctgaccgattcagtggcagcgggtctgggacagatttcactctcaccatcagcagcctgcaggctgaagatgtggcaaattattactgtcaccaatattacagtattccgtggacgttcggccaagggaccaaggtggaaatcaaa

SEQ ID NO: 68 - protein, 113 aa, synthetic construct
  REGION 1..113: note = Synthetic
DIVMTQSPDSLAVSLGERATINCKSSQSVFHTSNNKNYLVWYQQKPGQPPKLLLYWASTRESGVPDRFSGSGSGTDFTLTISSLQAEDVANYYCHQYYSIPWTFGQGTKVEIK

SEQ ID NO: 69 - DNA, 342 bp, synthetic construct; identical to SEQ ID NO: 45
  misc_feature 1..342: note = Synthetic
  source 1..342: mol_type = other DNA

SEQ ID NO: 70 - protein, 114 aa, synthetic construct; identical to SEQ ID NO: 46
  REGION 1..114: note = Synthetic

SEQ ID NO: 71 - DNA, 339 bp, synthetic construct
  misc_feature 1..339: note = Synthetic
  source 1..339: mol_type = other DNA
gacatcgtgatgacccagtctccagactccctggctgtgtctctgggcgagagggccaccatcaactgcaagtccagccagagtgtttttcacacctccaacaataagaactacttagcttggtaccagcagaaaccaggacagcctcctaagctgctcatttactgggcctctacccgggaatccggggtccctgaccgattcagtggcagcgggtctgggacagatttcactctcaccatcagcagcctgcaggctgaagatgtggcagtttattactgtcaccaatattacagtattccgtggacgttcggccaagggaccaaggtggaaatcaaa

SEQ ID NO: 72 - protein, 113 aa, synthetic construct
  REGION 1..113: note = Synthetic
DIVMTQSPDSLAVSLGERATINCKSSQSVFHTSNNKNYLAWYQQKPGQPPKLLIYWASTRESGVPDRFSGSGSGTDFTLTISSLQAEDVAVYYCHQYYSIPWTFGQGTKVEIK

SEQ ID NO: 73 - DNA, 354 bp, synthetic construct
  misc_feature 1..354: note = Synthetic
  source 1..354: mol_type = other DNA
gaagtgcagctggtgcagtctgggggaggcttggtacagcctggggggtccctgagactctcctgtgcagcctctggattcacctttaacaactatgccatgaactgggtccgccaggctccaggaaagggactggactgggtctcaactattagtggtagcggtggtactacaaactacgcagactccgtgaagggccgtttcattatttcccgagacagttccaaacacacgctgtatctgcaaatgaacagcctgagagccgaggacacggccgtatattactgtgcgaaagattctaactggggaaatttcgatctctggggccgtggcaccacggtcactgtctcctca

SEQ ID NO: 74 - protein, 118 aa, synthetic construct
  REGION 1..118: note = Synthetic
EVQLVQSGGGLVQPGGSLRLSCAASGFTFNNYAMNWVRQAPGKGLDWVSTISGSGGTTNYADSVKGRFIISRDSSKHTLYLQMNSLRAEDTAVYYCAKDSNWGNFDLWGRGTTVTVSS

SEQ ID NO: 75 - DNA, 24 bp, synthetic construct
  misc_feature 1..24: note = Synthetic
  source 1..24: mol_type = other DNA
ggattcacctttaacaactatgcc

SEQ ID NO: 76 - protein, 8 aa, synthetic construct
  REGION 1..8: note = Synthetic
GFTFNNYA

SEQ ID NO: 77 - DNA, 24 bp, synthetic construct
  misc_feature 1..24: note = Synthetic
  source 1..24: mol_type = other DNA
attagtggtagcggtggtactaca

SEQ ID NO: 78 - protein, 8 aa, synthetic construct
  REGION 1..8: note = Synthetic
ISGSGGTT

SEQ ID NO: 79 - DNA, 33 bp, synthetic construct
  misc_feature 1..33: note = Synthetic
  source 1..33: mol_type = other DNA
gcgaaagattctaactggggaaatttcgatctc

SEQ ID NO: 80 - protein, 11 aa, synthetic construct
  REGION 1..11: note = Synthetic
AKDSNWGNFDL

SEQ ID NO: 81 - DNA, 342 bp, synthetic construct
  misc_feature 1..342: note = Synthetic
  source 1..342: mol_type = other DNA
gacatccagatgacccagtctccagactccctggctgtgtctctgggcgagagggccaccatcaactgcaagtccagccagagtgttttatacaggtccaacaataggaacttcttaggttggtaccagcagaaaccagggcagcctcctaatctactcatttactgggcatctacccgggaatccggggtccctgaccgattcagtggcagcgggtctgggacagatttcactctcaccatcagcagcctgcaggctgaagatgtggcagtttattactgtcaacaatattatactactccgtacacttttggccaggggaccaaggtggaaatcaaacga

SEQ ID NO: 82 - protein, 114 aa, synthetic construct
  REGION 1..114: note = Synthetic
DIQMTQSPDSLAVSLGERATINCKSSQSVLYRSNNRNFLGWYQQKPGQPPNLLIYWASTRESGVPDRFSGSGSGTDFTLTISSLQAEDVAVYYCQQYYTTPYTFGQGTKVEIKR

SEQ ID NO: 83 - DNA, 36 bp, synthetic construct
  misc_feature 1..36: note = Synthetic
  source 1..36: mol_type = other DNA
cagagtgttttatacaggtccaacaataggaacttc

SEQ ID NO: 84 - protein, 12 aa, synthetic construct
  REGION 1..12: note = Synthetic
QSVLYRSNNRNF

SEQ ID NO: 85-86 - skipped (2 entries)

SEQ ID NO: 87 - DNA, 27 bp, synthetic construct
  misc_feature 1..27: note = Synthetic
  source 1..27: mol_type = other DNA
caacaatattatactactccgtacact

SEQ ID NO: 88 - protein, 9 aa, synthetic construct
  REGION 1..9: note = Synthetic
QQYYTTPYT

SEQ ID NO: 89 - DNA, 354 bp, synthetic construct
  misc_feature 1..354: note = Synthetic
  source 1..354: mol_type = other DNA
gaggtgcagctggtggagtctgggggaggcttggtacagcctggggggtccctgagactctcctgtgcagcctctggattcacctttaacaactatgccatgaactgggtccgccaggctccaggaaagggactggactgggtctcaactattagtggtagcggtggtactacaaactacgcagactccgtgaagggccgtttcattatttcccgagacagttccaaacacacgctgtatctgcaaatgaacagcctgagagccgaggacacggccgtatattactgtgcgaaagattctaactggggaaatttcgatctctggggccgtggcaccctggtcactgtctcctca

SEQ ID NO: 90 - protein, 118 aa, synthetic construct [sequence omitted]

SEQ ID NO: 91 - DNA, 339 bp, synthetic construct
  misc_feature 1..339: note = Synthetic
  source 1..339: mol_type = other DNA
gacatcgtgatgacccagtctccagactccctggctgtgtctctgggcgagagggccaccatcaactgcaagtccagccagagtgttttatacaggtccaacaataggaacttcttaggttggtaccagcagaaaccagggcagcctcctaatctactcatttactgggcatctacccgggaatccggggtccctgaccgattcagtggcagcgggtctgggacagatttcactctcaccatcagcagcctgcaggctgaagatgtggcagtttattactgtcaacaatattatactactccgtacacttttggccaggggaccaagctggagatcaaa

SEQ ID NO: 92 - protein, 113 aa, synthetic construct [sequence omitted]

SEQ ID NO: 93 - DNA, 354 bp, synthetic construct
  misc_feature 1..354: note = Synthetic
  source 1..354: mol_type = other DNA
gaggtgcagctggtggagtctgggggaggcttggtacagcctggggggtccctgagactctcctgtgcagcctctggattcacctttaacaactatgccatgagctgggtccgccaggctccagggaaggggctggagtgggtctcagctattagtggtagcggtggtactacatactacgcagactccgtgaagggccggttcaccatctccagagacaattccaagaacacgctgtatctgcaaatgaacagcctgagagccgaggacacggccgtatattactgtgcgaaagattctaactggggaaatttcgatctctggggccgtggcaccctggtcactgtctcctca

SEQ ID NO: 94 - protein, 118 aa, synthetic construct
  REGION 1..118: note = Synthetic
EVQLVESGGGLVQPGGSLRLSCAASGFTFNNYAMSWVRQAPGKGLEWVSAISGSGGTTYYADSVKGRFTISRDNSKNTLYLQMNSLRAEDTAVYYCAKDSNWGNFDLWGRGTLVTVSS

SEQ ID NO: 95 - DNA, 339 bp, synthetic construct
  misc_feature 1..339: note = Synthetic
  source 1..339: mol_type = other DNA
gacatcgtgatgacccagtctccagactccctggctgtgtctctgggcgagagggccaccatcaactgcaagtccagccagagtgttttatacaggtccaacaataggaacttcttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcaacaatattatactactccgtacacttttggccaggggaccaagctggagatcaaa

SEQ ID NO: 96 - protein, 113 aa, synthetic construct
  REGION 1..113: note = Synthetic
DIVMTQSPDSLAVSLGERATINCKSSQSVLYRSNNRNFLAWYQQKPGQPPKLLIYWASTRESGVPDRFSGSGSGTDFTLTISSLQAEDVAVYYCQQYYTTPYTFGQGTKLEIK

SEQ ID NO: 97 - DNA, 351 bp, synthetic construct
  misc_feature 1..351: note = Synthetic
  source 1..351: mol_type = other DNA
caggtgcagctggtgcagtctgggggaggcttggtacagcctggggggtccctgagactctcctgtgcagtctctggattcaccctcagtagctacgatatgcactgggtccgccaacctacaggaaaaggtctggagtgggtctcagctattggttctactggtgacacatactatccaggctccgtgaagggccgattcaccatctccagagaaaatgccaagaactccttgtatcttcaaatgaacagcctgagagccggggacacggctgtgtattactgtgcaagagagggatgggacgtaccctttgacttctggggccagggaaccctggtcaccgtctcctca

SEQ ID NO: 98 - protein, 117 aa, synthetic construct
  REGION 1..117: note = Synthetic
QVQLVQSGGGLVQPGGSLRLSCAVSGFTLSSYDMHWVRQPTGKGLEWVSAIGSTGDTYYPGSVKGRFTISRENAKNSLYLQMNSLRAGDTAVYYCAREGWDVPFDFWGQGTLVTVSS

SEQ ID NO: 99 - DNA, 24 bp, synthetic construct
  misc_feature 1..24: note = Synthetic
  source 1..24: mol_type = other DNA
ggattcaccctcagtagctacgat

SEQ ID NO: 100 - protein, 8 aa, synthetic construct
  REGION 1..8: note = Synthetic
GFTLSSYD

SEQ ID NO: 101 - DNA, 21 bp, synthetic construct
  misc_feature 1..21: note = Synthetic
  source 1..21: mol_type = other DNA
attggttctactggtgacaca

SEQ ID NO: 102 - protein, 7 aa, synthetic construct
  REGION 1..7: note = Synthetic
IGSTGDT

SEQ ID NO: 103 - DNA, 33 bp, synthetic construct
  misc_feature 1..33: note = Synthetic
  source 1..33: mol_type = other DNA
gcaagagagggatgggacgtaccctttgacttc

SEQ ID NO: 104 - protein, 11 aa, synthetic construct
  REGION 1..11: note = Synthetic
AREGWDVPFDF

SEQ ID NO: 105 - DNA, 324 bp, synthetic construct
  misc_feature 1..324: note = Synthetic
  source 1..324: mol_type = other DNA
gccatccagttgacccagtctccatcctccctgtctgcatctgtaggagacagagtcaccatcacttgccgggcaagtcaggacattagaaatgatttaggctggtatcagcagaaaccagggaaagcccctaagctcctgatctatgctgcatccagtttacaaagtggggtcccatcacggttcagcggcagtggatctggcacagatttcactctcaccatcagcagcctgcagcctgaagattttgcaacttattactgtctacaagattacaattacccgtggacgttcggccaagggaccaaggtggagatcaaacga

SEQ ID NO: 106 - protein, 108 aa, synthetic construct
  REGION 1..108: note = Synthetic
AIQLTQSPSSLSASVGDRVTITCRASQDIRNDLGWYQQKPGKAPKLLIYAASSLQSGVPSRFSGSGSGTDFTLTISSLQPEDFATYYCLQDYNYPWTFGQGTKVEIKR

SEQ ID NO: 107 - DNA, 18 bp, synthetic construct
  misc_feature 1..18: note = Synthetic
  source 1..18: mol_type = other DNA
caggacattagaaatgat

SEQ ID NO: 108 - protein, 6 aa, synthetic construct
  REGION 1..6: note = Synthetic
QDIRND

SEQ ID NO: 109-110 - skipped (2 entries)

SEQ ID NO: 111 - DNA, 27 bp, synthetic construct
  misc_feature 1..27: note = Synthetic
  source 1..27: mol_type = other DNA
ctacaagattacaattacccgtggacg

SEQ ID NO: 112 - protein, 9 aa, synthetic construct
  REGION 1..9: note = Synthetic
LQDYNYPWT

SEQ ID NO: 113 - DNA, 351 bp, synthetic construct
  misc_feature 1..351: note = Synthetic
  source 1..351: mol_type = other DNA
gaggtgcagctggtggagtctgggggaggcttggtacagcctggggggtccctgagactctcctgtgcagtctctggattcaccctcagtagctacgatatgcactgggtccgccaacctacaggaaaaggtctggagtgggtctcagctattggttctactggtgacacatactatccaggctccgtgaagggccgattcaccatctccagagaaaatgccaagaactccttgtatcttcaaatgaacagcctgagagccggggacacggctgtgtattactgtgcaagagagggatgggacgtaccctttgacttctggggccagggaaccctggtcaccgtctcctca

SEQ ID NO: 114 - protein, 117 aa, synthetic construct
  REGION 1..117: note = Synthetic
EVQLVESGGGLVQPGGSLRLSCAVSGFTLSSYDMHWVRQPTGKGLEWVSAIGSTGDTYYPGSVKGRFTISRENAKNSLYLQMNSLRAGDTAVYYCAREGWDVPFDFWGQGTLVTVSS

SEQ ID NO: 115 - DNA, 321 bp, synthetic construct
  misc_feature 1..321: note = Synthetic
  source 1..321: mol_type = other DNA
gccatccagatgacccagtctccatcctccctgtctgcatctgtaggagacagagtcaccatcacttgccgggcaagtcaggacattagaaatgatttaggctggtatcagcagaaaccagggaaagcccctaagctcctgatctatgctgcatccagtttacaaagtggggtcccatcacggttcagcggcagtggatctggcacagatttcactctcaccatcagcagcctgcagcctgaagattttgcaacttattactgtctacaagattacaattacccgtggacgttcggccaagggaccaaggtggaaatcaaa

SEQ ID NO: 116 - protein, 107 aa, synthetic construct
  REGION 1..107: note = Synthetic
AIQMTQSPSSLSASVGDRVTITCRASQDIRNDLGWYQQKPGKAPKLLIYAASSLQSGVPSRFSGSGSGTDFTLTISSLQPEDFATYYCLQDYNYPWTFGQGTKVEIK

SEQ ID NO: 117 - DNA, 351 bp, synthetic construct
  misc_feature 1..351: note = Synthetic
  source 1..351: mol_type = other DNA
gaggtgcagctggtggagtctgggggaggcttggtacagcctggggggtccctgagactctcctgtgcagcctctggattcaccctcagtagctacgatatgcactgggtccgccaagctacaggaaaaggtctggagtgggtctcagctattggttctactggtgacacatactatccaggctccgtgaagggccgattcaccatctccagagaaaatgccaagaactccttgtatcttcaaatgaacagcctgagagccggggacacggctgtgtattactgtgcaagagagggatgggacgtaccctttgacttctggggccagggaaccctggtcaccgtctcctca

SEQ ID NO: 118 - protein, 117 aa, synthetic construct
  REGION 1..117: note = Synthetic
EVQLVESGGGLVQPGGSLRLSCAASGFTLSSYDMHWVRQATGKGLEWVSAIGSTGDTYYPGSVKGRFTISRENAKNSLYLQMNSLRAGDTAVYYCAREGWDVPFDFWGQGTLVTVSS

SEQ ID NO: 119 - DNA, 321 bp, synthetic construct
  misc_feature 1..321: note = Synthetic
  source 1..321: mol_type = other DNA
gccatccagatgacccagtctccatcctccctgtctgcatctgtaggagacagagtcaccatcacttgccgggcaagtcaggacattagaaatgatttaggctggtatcagcagaaaccagggaaagcccctaagctcctgatctatgctgcatccagtttacaaagtggggtcccatcaaggttcagcggcagtggatctggcacagatttcactctcaccatcagcagcctgcagcctgaagattttgcaacttattactgtctacaagattacaattacccgtggacgttcggccaagggaccaaggtggaaatcaaa

SEQ ID NO: 120 - protein, 107 aa, synthetic construct; identical to SEQ ID NO: 116
  REGION 1..107: note = Synthetic

SEQ ID NO: 121 - DNA, 384 bp, synthetic construct
  misc_feature 1..384: note = Synthetic
  source 1..384: mol_type = other DNA
caggtgcagctgcaggagtcggggccaggactggtgaagccttcggagaccctgtccctcacctgcactgtctctggggactccatcaatacttactactggagctggttccggcagcccccagggaagggactggagtggattgggtatatctattatagtggaaccaccaactacaacccctccctcaagagtcgagtcaccatatcaatagacacgcccaggaaccagttctccctgaagctgatctctgtgaccgcagcggacacggccgtgtattactgtgcgagagagaggattactatgattcggggagttaccctctactattactcctacggtatggacgtctggggccaagggaccacggtcaccgtctcctca

SEQ ID NO: 122 - protein, 128 aa, synthetic construct
  REGION 1..128: note = Synthetic
QVQLQESGPGLVKPSETLSLTCTVSGDSINTYYWSWFRQPPGKGLEWIGYIYYSGTTNYNPSLKSRVTISIDTPRNQFSLKLISVTAADTAVYYCARERITMIRGVTLYYYSYGMDVWGQGTTVTVSS

SEQ ID NO: 123 - DNA, 24 bp, synthetic construct
  misc_feature 1..24: note = Synthetic
  source 1..24: mol_type = other DNA
ggggactccatcaatacttactac

SEQ ID NO: 124 - protein, 8 aa, synthetic construct
  REGION 1..8: note = Synthetic
GDSINTYY

SEQ ID NO: 125 - DNA, 21 bp, synthetic construct
  misc_feature 1..21: note = Synthetic
  source 1..21: mol_type = other DNA
atctattatagtggaaccacc

SEQ ID NO: 126 - protein, 7 aa, synthetic construct
  REGION 1..7: note = Synthetic
IYYSGTT

SEQ ID NO: 127 - DNA, 66 bp, synthetic construct
  misc_feature 1..66: note = Synthetic
  source 1..66: mol_type = other DNA
gcgagagagaggattactatgattcggggagttaccctctactattactcctacggtatggacgtc

SEQ ID NO: 128 - protein, 22 aa, synthetic construct
  REGION 1..22: note = Synthetic
ARERITMIRGVTLYYYSYGMDV

SEQ ID NO: 129 - DNA, 324 bp, synthetic construct
  misc_feature 1..324: note = Synthetic
  source 1..324: mol_type = other DNA
gacatccagatgacccagtctccatccttcctgtctgcatctgtaggagacagagtcaccatcacttgctgggccagtcaggacattagcagttatttagcctggtatcagcaaaaaccagggatagcccctaagctcctgatctatgctgcatccactttgcaaagtggggtcccatcaaggttcggcggcagtggatctgggacagaattcactctcacaatcagcagcctgcagcctgaagattttgcaacttattactgtcaacagcttaatagttaccctcggacgttcggccaagggaccaaggtggaaatcaaacga

SEQ ID NO: 130 - protein, 108 aa, synthetic construct
  REGION 1..108: note = Synthetic
DIQMTQSPSFLSASVGDRVTITCWASQDISSYLAWYQQKPGIAPKLLIYAASTLQSGVPSRFGGSGSGTEFTLTISSLQPEDFATYYCQQLNSYPRTFGQGTKVEIKR

SEQ ID NO: 131 - DNA, 18 bp, synthetic construct
  misc_feature 1..18: note = Synthetic
  source 1..18: mol_type = other DNA
caggacattagcagttat

SEQ ID NO: 132 - protein, 6 aa, synthetic construct
  REGION 1..6: note = Synthetic
QDISSY

SEQ ID NO: 133-134 - skipped (2 entries)

SEQ ID NO: 135 - DNA, 27 bp, synthetic construct
  misc_feature 1..27: note = Synthetic
  source 1..27: mol_type = other DNA
caacagcttaatagttaccctcggacg

SEQ ID NO: 136 - protein, 9 aa, synthetic construct
  REGION 1..9: note = Synthetic
QQLNSYPRT

SEQ ID NO: 137 - DNA, 384 bp, synthetic construct; identical to SEQ ID NO: 121
  misc_feature 1..384: note = Synthetic
  source 1..384: mol_type = other DNA

SEQ ID NO: 138 - protein, 128 aa, synthetic construct; identical to SEQ ID NO: 122
  REGION 1..128: note = Synthetic

SEQ ID NO: 139 - DNA, 321 bp, synthetic construct
  misc_feature 1..321: note = Synthetic
  source 1..321: mol_type = other DNA
gacatccagatgacccagtctccatccttcctgtctgcatctgtaggagacagagtcaccatcacttgctgggccagtcaggacattagcagttatttagcctggtatcagcaaaaaccagggatagcccctaagctcctgatctatgctgcatccactttgcaaagtggggtcccatcaaggttcggcggcagtggatctgggacagaattcactctcacaatcagcagcctgcagcctgaagattttgcaacttattactgtcaacagcttaatagttaccctcggacgttcggccaagggaccaaggtggaaatcaaa

SEQ ID NO: 140 - protein, 107 aa, synthetic construct
  REGION 1..107: note = Synthetic
DIQMTQSPSFLSASVGDRVTITCWASQDISSYLAWYQQKPGIAPKLLIYAASTLQSGVPSRFGGSGSGTEFTLTISSLQPEDFATYYCQQLNSYPRTFGQGTKVEIK

SEQ ID NO: 141 - DNA, 384 bp, synthetic construct
  misc_feature 1..384: note = Synthetic
  source 1..384: mol_type = other DNA
caggtgcagctgcaggagtcgggcccaggactggtgaagccttcggagaccctgtccctcacctgcactgtctctggggactccatcaatacttactactggagctggatccggcagcccccagggaagggactggagtggattgggtatatctattatagtggaaccaccaactacaacccctccctcaagagtcgagtcaccatatcagtagacacgtccaagaaccagttctccctgaagctgagctctgtgaccgctgcggacacggccgtgtattactgtgcgagagagaggattactatgattcggggagttaccctctactattactcctacggtatggacgtctggggccaagggaccacggtcaccgtctcctca

SEQ ID NO: 142 - protein, 128 aa, synthetic construct
  REGION 1..128: note = Synthetic
QVQLQESGPGLVKPSETLSLTCTVSGDSINTYYWSWIRQPPGKGLEWIGYIYYSGTTNYNPSLKSRVTISVDTSKNQFSLKLSSVTAADTAVYYCARERITMIRGVTLYYYSYGMDVWGQGTTVTVSS

SEQ ID NO: 143 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgccgggcaagtcaggacattagcagttatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtcaacagcttaatagttaccctcggacgttcggccaagggaccaaggtggaaatcaaa

SEQ ID NO: 144 - protein, 107 aa, synthetic construct
  REGION 1..107: note = Synthetic
DIQMTQSPSSLSASVGDRVTITCRASQDISSYLGWYQQKPGKAPKRLIYAASSLQSGVPSRFSGSGSGTEFTLTISSLQPEDFATYYCQQLNSYPRTFGQGTKVEIK

SEQ ID NO: 145 - DNA, 378 bp, synthetic construct
  misc_feature 1..378: note = Synthetic
  source 1..378: mol_type = other DNA
caggtgcagctggtgcagtctggagctgaggtgaagaagcctggggcctcagtgaaggtctcctgcaaggcttctggttacacctttaccaactatggtatcagctgggtgcgacaggcccctggacaaggacttgagttaatgggatggattagtggttacaatggtaacacaaactatgcacaagaactccaggccagagtcaccatgaccacagacacatccacgagcacagcctacatggagctgaggaacctgagatctgacgacacggccgtatattactgtgcgagagatagagtcgttgtagcagctgctaattactacttttattctatggacgtctggggccaagggaccacggtcaccgtctcctca

SEQ ID NO: 146 - protein, 126 aa, synthetic construct
  REGION 1..126: note = Synthetic
QVQLVQSGAEVKKPGASVKVSCKASGYTFTNYGISWVRQAPGQGLELMGWISGYNGNTNYAQELQARVTMTTDTSTSTAYMELRNLRSDDTAVYYCARDRVVVAAANYYFYSMDVWGQGTTVTVSS

SEQ ID NO: 147 - DNA, 24 bp, synthetic construct
  misc_feature 1..24: note = Synthetic
  source 1..24: mol_type = other DNA
ggttacacctttaccaactatggt

SEQ ID NO: 148 - protein, 8 aa, synthetic construct
  REGION 1..8: note = Synthetic
GYTFTNYG

SEQ ID NO: 149 - DNA, 24 bp, synthetic construct
  misc_feature 1..24: note = Synthetic
  source 1..24: mol_type = other DNA
attagtggttacaatggtaacaca

SEQ ID NO: 150 - protein, 8 aa, synthetic construct
  REGION 1..8: note = Synthetic
ISGYNGNT

SEQ ID NO: 151 - DNA, 57 bp, synthetic construct
  misc_feature 1..57: note = Synthetic
  source 1..57: mol_type = other DNA
gcgagagatagagtcgttgtagcagctgctaattactacttttattctatggacgtc

SEQ ID NO: 152 - protein, 19 aa, synthetic construct
  REGION 1..19: note = Synthetic
ARDRVVVAAANYYFYSMDV

SEQ ID NO: 153 - DNA, 339 bp, synthetic construct
  misc_feature 1..339: note = Synthetic
  source 1..339: mol_type = other DNA
gccatccagatgacccagtctccactctccctgtccgtcacccttggacagccggcctccatctcctgcaggtctagtcaaagcctcgtatacagtgatggagacacctacttgaattggtttcagcagaggccaggccaatctccaaggcgcctaatttataaggtttctaaccgggactctggggtcccagacagattcagcggcagtgggtcaggcactgctttcacactgaaaatcagcggggtggaggccgaggatgttggggtttactactgcatgcaagctacacactggcctcggacgttcggccaagggaccaaggtggaaatcaaacga

SEQ ID NO: 154 - protein, 113 aa, synthetic construct
  REGION 1..113: note = Synthetic
AIQMTQSPLSLSVTLGQPASISCRSSQSLVYSDGDTYLNWFQQRPGQSPRRLIYKVSNRDSGVPDRFSGSGSGTAFTLKISGVEAEDVGVYYCMQATHWPRTFGQGTKVEIKR

SEQ ID NO: 155 - DNA, 33 bp, synthetic construct
  misc_feature 1..33: note = Synthetic
  source 1..33: mol_type = other DNA
caaagcctcgtatacagtgatggagacacctac

SEQ ID NO: 156 - protein, 11 aa, synthetic construct
  REGION 1..11: note = Synthetic
QSLVYSDGDTY

SEQ ID NO: 157-158 - skipped (2 entries)

SEQ ID NO: 159 - DNA, 27 bp, synthetic construct
  misc_feature 1..27: note = Synthetic
  source 1..27: mol_type = other DNA
atgcaagctacacactggcctcggacg

SEQ ID NO: 160 - protein, 9 aa, synthetic construct
  REGION 1..9: note = Synthetic
MQATHWPRT

SEQ ID NO: 161 - DNA, 378 bp, synthetic construct
  misc_feature 1..378: note = Synthetic
  source 1..378: mol_type = other DNA
caggttcagctggtgcagtctggagctgaggtgaagaagcctggggcctcagtgaaggtctcctgcaaggcttctggttacacctttaccaactatggtatcagctgggtgcgacaggcccctggacaaggacttgagttaatgggatggattagtggttacaatggtaacacaaactatgcacaagaactccaggccagagtcaccatgaccacagacacatccacgagcacagcctacatggagctgaggaacctgagatctgacgacacggccgtatattactgtgcgagagatagagtcgttgtagcagctgctaattactacttttattctatggacgtctggggccaagggaccacggtcaccgtctcctca

SEQ ID NO: 162 - protein, 126 aa, synthetic construct; identical to SEQ ID NO: 146
  REGION 1..126: note = Synthetic

SEQ ID NO: 163 - DNA, 336 bp, synthetic construct
  misc_feature 1..336: note = Synthetic
  source 1..336: mol_type = other DNA
gatgttgtgatgactcagtctccactctccctgtccgtcacccttggacagccggcctccatctcctgcaggtctagtcaaagcctcgtatacagtgatggagacacctacttgaattggtttcagcagaggccaggccaatctccaaggcgcctaatttataaggtttctaaccgggactctggggtcccagacagattcagcggcagtgggtcaggcactgctttcacactgaaaatcagcggggtggaggccgaggatgttggggtttactactgcatgcaagctacacactggcctcggacgttcggccaagggaccaaggtggaaatcaaa

SEQ ID NO: 164 - protein, 112 aa, synthetic construct
  REGION 1..112: note = Synthetic
DVVMTQSPLSLSVTLGQPASISCRSSQSLVYSDGDTYLNWFQQRPGQSPRRLIYKVSNRDSGVPDRFSGSGSGTAFTLKISGVEAEDVGVYYCMQATHWPRTFGQGTKVEIK

SEQ ID NO: 165 - DNA, 378 bp, synthetic construct
  misc_feature 1..378: note = Synthetic
  source 1..378: mol_type = other DNA
caggttcagctggtgcagtctggagctgaggtgaagaagcctggggcctcagtgaaggtctcctgcaaggcttctggttacacctttaccaactatggtatcagctgggtgcgacaggcccctggacaagggcttgagtggatgggatggattagtggttacaatggtaacacaaactatgcacagaagctccagggcagagtcaccatgaccacagacacatccacgagcacagcctacatggagctgaggagcctgagatctgacgacacggccgtgtattactgtgcgagagatagagtcgttgtagcagctgctaattactacttttattctatggacgtctggggccaagggaccacggtcaccgtctcctca

SEQ ID NO: 166 - protein, 126 aa, synthetic construct
  REGION 1..126: note = Synthetic
QVQLVQSGAEVKKPGASVKVSCKASGYTFTNYGISWVRQAPGQGLEWMGWISGYNGNTNYAQKLQGRVTMTTDTSTSTAYMELRSLRSDDTAVYYCARDRVVVAAANYYFYSMDVWGQGTTVTVSS

SEQ ID NO: 167 - DNA, 336 bp, synthetic construct
  misc_feature 1..336: note = Synthetic
  source 1..336: mol_type = other DNA
gatgttgtgatgactcagtctccactctccctgcccgtcacccttggacagccggcctccatctcctgcaggtctagtcaaagcctcgtatacagtgatggagacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaagctacacactggcctcggacgttcggccaagggaccaaggtggaaatcaaa

SEQ ID NO: 168 - protein, 112 aa, synthetic construct
  REGION 1..112: note = Synthetic
DVVMTQSPLSLPVTLGQPASISCRSSQSLVYSDGDTYLNWFQQRPGQSPRRLIYKVSNRDSGVPDRFSGSGSGTDFTLKISRVEAEDVGVYYCMQATHWPRTFGQGTKVEIK

SEQ ID NO: 169 - DNA, 375 bp, synthetic construct
  misc_feature 1..375: note = Synthetic
  source 1..375: mol_type = other DNA
caggtccacttgaaggagtctggtcctacgctggtgaaacccacacagaccctcacgctgacctgcaccttctctggattctcactcatcactagtggagtgggtgtgggctggattcgtcagccccccggaaaggccctggagtggcttgcactcatttattggaatggtgataagcgctacagcccatctctgaagagcaggctcaccatcaccaaggacacctccaaaaaccaggtggtccttacaatgaccaacatggaccctgtggacacagccacatattactgtgcacacaggataactgaaactagttactacttctactacggtatggacgtctggggccaagggaccacggtcaccgtctcctca

SEQ ID NO: 170 - protein, 125 aa, synthetic construct
  REGION 1..125: note = Synthetic
QVHLKESGPTLVKPTQTLTLTCTFSGFSLITSGVGVGWIRQPPGKALEWLALIYWNGDKRYSPSLKSRLTITKDTSKNQVVLTMTNMDPVDTATYYCAHRITETSYYFYYGMDVWGQGTTVTVSS

SEQ ID NO: 171 - DNA, 30 bp, synthetic construct
  misc_feature 1..30: note = Synthetic
  source 1..30: mol_type = other DNA
ggattctcactcatcactagtggagtgggt

SEQ ID NO: 172 - protein, 10 aa, synthetic construct
  REGION 1..10: note = Synthetic
GFSLITSGVG

SEQ ID NO: 173 - DNA, 21 bp, synthetic construct
  misc_feature 1..21: note = Synthetic
  source 1..21: mol_type = other DNA
atttattggaatggtgataag

SEQ ID NO: 174 - protein, 7 aa, synthetic construct
  REGION 1..7: note = Synthetic
IYWNGDK

SEQ ID NO: 175 - DNA, 51 bp, synthetic construct
  misc_feature 1..51: note = Synthetic
  source 1..51: mol_type = other DNA
gcacacaggataactgaaactagttactacttctactacggtatggacgtc

SEQ ID NO: 176 - protein, 17 aa, synthetic construct
  REGION 1..17: note = Synthetic
AHRITETSYYFYYGMDV

SEQ ID NO: 177 - DNA, 339 bp, synthetic construct
  misc_feature 1..339: note = Synthetic
  source 1..339: mol_type = other DNA
gacatccagatgacccagtctccactctccctgcccgtcacccctggagagccggcctccatctcctgcaggtctagtcagagcctcctgcatagtcatggatacgactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgctcactttcggcggagggaccaaggtggaaatcaaacga

SEQ ID NO: 178 - protein, 113 aa, synthetic construct
  REGION 1..113: note = Synthetic
DIQMTQSPLSLPVTPGEPASISCRSSQSLLHSHGYDYLDWYLQKPGQSPQLLIYLGSNRASGVPDRFSGSGSGTDFTLKISRVEAEDVGVYYCMQALQTPLTFGGGTKVEIKR

SEQ ID NO: 179 - DNA, 33 bp, synthetic construct
  misc_feature 1..33: note = Synthetic
  source 1..33: mol_type = other DNA
cagagcctcctgcatagtcatggatacgactat

SEQ ID NO: 180 - protein, 11 aa, synthetic construct
  REGION 1..11: note = Synthetic
QSLLHSHGYDY

SEQ ID NO: 181-182 - skipped (2 entries)

SEQ ID NO: 183 - DNA, 27 bp, synthetic construct
  misc_feature 1..27: note = Synthetic
  source 1..27: mol_type = other DNA
atgcaagctctacaaactccgctcact

SEQ ID NO: 184 - protein, 9 aa, synthetic construct
  REGION 1..9: note = Synthetic
MQALQTPLT

SEQ ID NO: 185 - DNA, 375 bp, synthetic construct
  misc_feature 1..375: note = Synthetic
  source 1..375: mol_type = other DNA
cagatcaccttgaaggagtctggtcctacgctggtgaaacccacacagaccctcacgctgacctgcaccttctctggattctcactcatcactagtggagtgggtgtgggctggattcgtcagccccccggaaaggccctggagtggcttgcactcatttattggaatggtgataagcgctacagcccatctctgaagagcaggctcaccatcaccaaggacacctccaaaaaccaggtggtccttacaatgaccaacatggaccctgtggacacagccacatattactgtgcacacaggataactgaaactagttactacttctactacggtatggacgtctggggccaagggaccacggtcaccgtctcctca

SEQ ID NO: 186 - protein, 125 aa, synthetic construct
  REGION 1..125: note = Synthetic
QITLKESGPTLVKPTQTLTLTCTFSGFSLITSGVGVGWIRQPPGKALEWLALIYWNGDKRYSPSLKSRLTITKDTSKNQVVLTMTNMDPVDTATYYCAHRITETSYYFYYGMDVWGQGTTVTVSS

SEQ ID NO: 187 - DNA, 336 bp, synthetic construct
  misc_feature 1..336: note = Synthetic
  source 1..336: mol_type = other DNA
gatattgtgatgactcagtctccactctccctgcccgtcacccctggagagccggcctccatctcctgcaggtctagtcagagcctcctgcatagtcatggatacgactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgctcactttcggcggagggaccaaggtggagatcaaa

SEQ ID NO: 188 - protein, 112 aa, synthetic construct
  REGION 1..112: note = Synthetic
DIVMTQSPLSLPVTPGEPASISCRSSQSLLHSHGYDYLDWYLQKPGQSPQLLIYLGSNRASGVPDRFSGSGSGTDFTLKISRVEAEDVGVYYCMQALQTPLTFGGGTKVEIK

SEQ ID NO: 189 - DNA, 375 bp, synthetic construct
  misc_feature 1..375: note = Synthetic
  source 1..375: mol_type = other DNA
cagatcaccttgaaggagtctggtcctacgctggtgaaacccacacagaccctcacgctgacctgcaccttctctggattctcactcatcactagtggagtgggtgtgggctggatccgtcagcccccaggaaaggccctggagtggcttgcactcatttattggaatggtgataagcgctacagcccatctctgaagagcaggctcaccatcaccaaggacacctccaaaaaccaggtggtccttacaatgaccaacatggaccctgtggacacagccacatattactgtgcacacaggataactgaaactagttactacttctactacggtatggacgtctggggccaagggaccacggtcaccgtctcctca

SEQ ID NO: 190 - protein, 125 aa, synthetic construct; identical to SEQ ID NO: 186
  REGION 1..125: note = Synthetic

SEQ ID NO: 191 - DNA, 336 bp, synthetic construct; identical to SEQ ID NO: 187
  misc_feature 1..336: note = Synthetic
  source 1..336: mol_type = other DNA

SEQ ID NO: 192 - protein, 112 aa, synthetic construct; identical to SEQ ID NO: 188
  REGION 1..112: note = Synthetic

SEQ ID NO: 193 - DNA, 375 bp, synthetic construct
  misc_feature 1..375: note = Synthetic
  source 1..375: mol_type = other DNA
cagatcaccttgaaggagtctggtcctactctggtgaaaccctcacagaccctcacgctgacctgcaccttctctgggttctcactcagcactagtggagtgggtgtgggctggatccgtcagcccccaggaaaggccctggagtggcttgcactcatttattggaattctgataagcgctacagcccatctctgaagagcaggctcaccatcaccaaggacacctccaaaaaccaggtagtccttacaatgaccaacatggaccctgtggacacagccacatattactgtgcacacagacatgacagctcgtcctactacttctactacggtatggacgtctggggccaagggatcacggtcaccgtctcctca

SEQ ID NO: 194 - protein, 125 aa, synthetic construct
  REGION 1..125: note = Synthetic
QITLKESGPTLVKPSQTLTLTCTFSGFSLSTSGVGVGWIRQPPGKALEWLALIYWNSDKRYSPSLKSRLTITKDTSKNQVVLTMTNMDPVDTATYYCAHRHDSSSYYFYYGMDVWGQGITVTVSS

SEQ ID NO: 195 - DNA, 30 bp, synthetic construct
  misc_feature 1..30: note = Synthetic
  source 1..30: mol_type = other DNA
gggttctcactcagcactagtggagtgggt

SEQ ID NO: 196 - protein, 10 aa, synthetic construct
  REGION 1..10: note = Synthetic
GFSLSTSGVG

SEQ ID NO: 197 - DNA, 21 bp, synthetic construct
  misc_feature 1..21: note = Synthetic
  source 1..21: mol_type = other DNA
atttattggaattctgataag

SEQ ID NO: 198 - protein, 7 aa, synthetic construct
  REGION 1..7: note = Synthetic
IYWNSDK

SEQ ID NO: 199 - DNA, 51 bp, synthetic construct
  misc_feature 1..51: note = Synthetic
  source 1..51: mol_type = other DNA
gcacacagacatgacagctcgtcctactacttctactacggtatggacgtc

SEQ ID NO: 200 - protein, 17 aa, synthetic construct
  REGION 1..17: note = Synthetic
AHRHDSSSYYFYYGMDV

SEQ ID NO: 201 - DNA, 339 bp, synthetic construct
  misc_feature 1..339: note = Synthetic
  source 1..339: mol_type = other DNA
gacatccagatgacccagtctccgctctccctgcccgtcacccctggagagccggcctccatctcctgcaggtctagtcagagcctcctccatagtcatggatacaactatttggattggtacctgcagaagccagggcagtctccacaactcctgatctatttgggttctaatcgggcctccggggtccctgacaggttcagtggcggtggatcaggcacagattttacactgaaaatcagcagagtggaggctgaggatgttgggatttattactgcatgcaagctctacagactcctctcactttcggcggagggaccaaggtggagatcaaacga

SEQ ID NO: 202 - protein, 113 aa, synthetic construct
  REGION 1..113: note = Synthetic
DIQMTQSPLSLPVTPGEPASISCRSSQSLLHSHGYNYLDWYLQKPGQSPQLLIYLGSNRASGVPDRFSGGGSGTDFTLKISRVEAEDVGIYYCMQALQTPLTFGGGTKVEIKR

SEQ ID NO: 203 - DNA, 33 bp, synthetic construct
  misc_feature 1..33: note = Synthetic
  source 1..33: mol_type = other DNA
cagagcctcctccatagtcatggatacaactat

SEQ ID NO: 204 - protein, 11 aa, synthetic construct
  REGION 1..11: note = Synthetic
QSLLHSHGYNY

SEQ ID NO: 205-206 - skipped (2 entries)

SEQ ID NO: 207 - DNA, 27 bp, synthetic construct
  misc_feature 1..27: note = Synthetic
  source 1..27: mol_type = other DNA
atgcaagctctacagactcctctcact

SEQ ID NO: 208 - protein, 9 aa, synthetic construct
  REGION 1..9: note = Synthetic
MQALQTPLT

SEQ ID NO: 209 - DNA, 375 bp, synthetic construct
  misc_feature 1..375: note = Synthetic
  source 1..375: mol_type = other DNA
cagatcaccttgaaggagtctggtcctactctggtgaaaccctcacagaccctcacgctgacctgcaccttctctgggttctcactcagcactagtggagtgggtgtgggctggatccgtcagcccccaggaaaggccctggagtggcttgcactcatttattggaattctgataagcgctacagcccatctctgaagagcaggctcaccatcaccaaggacacctccaaaaaccaggtagtccttacaatgaccaacatggaccctgtggacacagccacatattactgtgcacacagacatgacagctcgtcctactacttctactacggtatggacgtctggggccaagggaccacggtcaccgtctcctca

SEQ ID NO: 210 - protein, 125 aa, synthetic construct
  REGION 1..125: note = Synthetic
QITLKESGPTLVKPSQTLTLTCTFSGFSLSTSGVGVGWIRQPPGKALEWLALIYWNSDKRYSPSLKSRLTITKDTSKNQVVLTMTNMDPVDTATYYCAHRHDSSSYYFYYGMDVWGQGTTVTVSS

SEQ ID NO: 211 - DNA, 336 bp, synthetic construct
  misc_feature 1..336: note = Synthetic
  source 1..336: mol_type = other DNA
gatattgtgatgactcagtctccgctctccctgcccgtcacccctggagagccggcctccatctcctgcaggtctagtcagagcctcctccatagtcatggatacaactatttggattggtacctgcagaagccagggcagtctccacaactcctgatctatttgggttctaatcgggcctccggggtccctgacaggttcagtggcggtggatcaggcacagattttacactgaaaatcagcagagtggaggctgaggatgttgggatttattactgcatgcaagctctacagactcctctcactttcggcggagggaccaaggtggagatcaaa

SEQ ID NO: 212 - protein, 112 aa, synthetic construct
  REGION 1..112: note = Synthetic
DIVMTQSPLSLPVTPGEPASISCRSSQSLLHSHGYNYLDWYLQKPGQSPQLLIYLGSNRASGVPDRFSGGGSGTDFTLKISRVEAEDVGIYYCMQALQTPLTFGGGTKVEIK

SEQ ID NO: 213 - DNA, 375 bp, synthetic construct
  misc_feature 1..375: note = Synthetic
  source 1..375: mol_type = other DNA
cagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttattggaattctgataagcgctacagcccatctctgaagagcaggctcaccatcaccaaggacacctccaaaaaccaggtggtccttacaatgaccaacatggaccctgtggacacagccacatattactgtgcacacagacatgacagctcgtcctactacttctactacggtatggacgtctggggccaagggaccacggtcaccgtctcctca

SEQ ID NO: 214 - protein, 125 aa, synthetic construct
  REGION 1..125: note = Synthetic
QITLKESGPTLVKPTQTLTLTCTFSGFSLSTSGVGVGWIRQPPGKALEWLALIYWNSDKRYSPSLKSRLTITKDTSKNQVVLTMTNMDPVDTATYYCAHRHDSSSYYFYYGMDVWGQGTTVTVSS

SEQ ID NO: 215 - DNA, 336 bp, synthetic construct
  misc_feature 1..336: note = Synthetic
  source 1..336: mol_type = other DNA
gatattgtgatgactcagtctccactctccctgcccgtcacccctggagagccggcctccatctcctgcaggtctagtcagagcctcctccatagtcatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacagactcctctcactttcggcggagggaccaaggtggagatcaaa

SEQ ID NO: 216 - protein, 112 aa, synthetic construct
  REGION 1..112: note = Synthetic
DIVMTQSPLSLPVTPGEPASISCRSSQSLLHSHGYNYLDWYLQKPGQSPQLLIYLGSNRASGVPDRFSGSGSGTDFTLKISRVEAEDVGVYYCMQALQTPLTFGGGTKVEIK

SEQ ID NO: 217 - DNA, 381 bp, synthetic construct
  misc_feature 1..381: note = Synthetic
  source 1..381: mol_type = other DNA
gagatgcaactggtggagtctgggggaggcttggtccagcctggggggtccctgagactctcctgtgcagcctctggattcacctttagtagtcactggatgaagtgggtccgccaggctccagggaaggggctggagtgggtggccaacataaaccaagatggaagtgagaaatactatgtggactctgtgaagggccgattcaccatctccagagacaacgccaagaactcactgtttctgcaaatgaacagcctgagagccgaggacacggctgtgtattactgtgcgagagatattgtactaatggtctatgatatggactactactactacggtatggacgtctggggccaagggaccacggtcaccgtctcctca

SEQ ID NO: 218 - protein, 127 aa, synthetic construct
  REGION 1..127: note = Synthetic
EMQLVESGGGLVQPGGSLRLSCAASGFTFSSHWMKWVRQAPGKGLEWVANINQDGSEKYYVDSVKGRFTISRDNAKNSLFLQMNSLRAEDTAVYYCARDIVLMVYDMDYYYYGMDVWGQGTTVTVSS

SEQ ID NO: 219 - DNA, 24 bp, synthetic construct
  misc_feature 1..24: note = Synthetic
  source 1..24: mol_type = other DNA
ggattcacctttagtagtcactgg

SEQ ID NO: 220 - protein, 8 aa, synthetic construct
  REGION 1..8: note = Synthetic
GFTFSSHW

SEQ ID NO: 221 - DNA, 24 bp, synthetic construct
  misc_feature 1..24: note = Synthetic
  source 1..24: mol_type = other DNA
ataaaccaagatggaagtgagaaa

SEQ ID NO: 222 - protein, 8 aa, synthetic construct
  REGION 1..8: note = Synthetic
INQDGSEK

SEQ ID NO: 223 - DNA, 60 bp, synthetic construct
  misc_feature 1..60: note = Synthetic
  source 1..60: mol_type = other DNA
gcgagagatattgtactaatggtctatgatatggactactactactacggtatggacgtc

SEQ ID NO: 224 - protein, 20 aa, synthetic construct
  REGION 1..20: note = Synthetic
ARDIVLMVYDMDYYYYGMDV

SEQ ID NO: 225 - DNA, 336 bp, synthetic construct
  misc_feature 1..336: note = Synthetic
  source 1..336: mol_type = other DNA
gatattgtgatgactcagtctccactctccctgcccgtcacccctggagagccggcctccatctcctgcaggtctagtcagagcctcctgcatagtaatggaaacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaaactctacaaactccgctcactttcggcggagggaccaaggtggagatcaaa

SEQ ID NO: 226 - protein, 112 aa, synthetic construct
  REGION 1..112: note = Synthetic
DIVMTQSPLSLPVTPGEPASISCRSSQSLLHSNGNNYLDWYLQKPGQSPQLLIYLGSNRASGVPDRFSGSGSGTDFTLKISRVEAEDVGVYYCMQTLQTPLTFGGGTKVEIK

SEQ ID NO: 227 - DNA, 33 bp, synthetic construct
  misc_feature 1..33: note = Synthetic
  source 1..33: mol_type = other DNA
cagagcctcctgcatagtaatggaaacaactat

SEQ ID NO: 228 - protein, 11 aa, synthetic construct
  REGION 1..11: note = Synthetic
QSLLHSNGNNY

SEQ ID NO: 229-230 - skipped (2 entries)

SEQ ID NO: 231 - DNA, 27 bp, synthetic construct
  misc_feature 1..27: note = Synthetic
  source 1..27: mol_type = other DNA
atgcaaactctacaaactccgctcact

SEQ ID NO: 232 - protein, 9 aa, synthetic construct
  REGION 1..9: note = Synthetic
MQTLQTPLT

SEQ ID NO: 233 - DNA, 381 bp, synthetic construct
  misc_feature 1..381: note = Synthetic
  source 1..381: mol_type = other DNA
gaggtgcagctggtggagtctgggggaggcttggtccagcctggggggtccctgagactctcctgtgcagcctctggattcacctttagtagtcactggatgaagtgggtccgccaggctccagggaaggggctggagtgggtggccaacataaaccaagatggaagtgagaaatactatgtggactctgtgaagggccgattcaccatctccagagacaacgccaagaactcactgtttctgcaaatgaacagcctgagagccgaggacacggctgtgtattactgtgcgagagatattgtactaatggtctatgatatggactactactactacggtatggacgtctggggccaagggaccacggtcaccgtctcctca

SEQ ID NO: 234 - protein, 127 aa, synthetic construct
  REGION 1..127: note = Synthetic
EVQLVESGGGLVQPGGSLRLSCAASGFTFSSHWMKWVRQAPGKGLEWVANINQDGSEKYYVDSVKGRFTISRDNAKNSLFLQMNSLRAEDTAVYYCARDIVLMVYDMDYYYYGMDVWGQGTTVTVSS

SEQ ID NO: 235 - DNA, 336 bp, synthetic construct; identical to SEQ ID NO: 225
  misc_feature 1..336: note = Synthetic
  source 1..336: mol_type = other DNA

SEQ ID NO: 236 - protein, 112 aa, synthetic construct; identical to SEQ ID NO: 226
  REGION 1..112: note = Synthetic

SEQ ID NO: 237 - DNA, 381 bp, synthetic construct
  misc_feature 1..381: note = Synthetic
  source 1..381: mol_type = other DNA
gaggtgcagctggtggagtctgggggaggcttggtccagcctggggggtccctgagactctcctgtgcagcctctggattcacctttagtagtcactggatgagctgggtccgccaggctccagggaaggggctggagtgggtggccaacataaaccaagatggaagtgagaaatactatgtggactctgtgaagggccgattcaccatctccagagacaacgccaagaactcactgtatctgcaaatgaacagcctgagagccgaggacacggctgtgtattactgtgcgagagatattgtactaatggtctatgatatggactactactactacggtatggacgtctgggggcaagggaccacggtcaccgtctcctca

SEQ ID NO: 238 - protein, 127 aa, synthetic construct
  REGION 1..127: note = Synthetic
EVQLVESGGGLVQPGGSLRLSCAASGFTFSSHWMSWVRQAPGKGLEWVANINQDGSEKYYVDSVKGRFTISRDNAKNSLYLQMNSLRAEDTAVYYCARDIVLMVYDMDYYYYGMDVWGQGTTVTVSS

SEQ ID NO: 239 - DNA, 336 bp, synthetic construct; identical to SEQ ID NO: 225
  misc_feature 1..336: note = Synthetic
  source 1..336: mol_type = other DNA

SEQ ID NO: 240 - protein, 112 aa, synthetic construct; identical to SEQ ID NO: 226
  REGION 1..112: note = Synthetic

SEQ ID NO: 241 - DNA, 381 bp, synthetic construct
  misc_feature 1..381: note = Synthetic
  source 1..381: mol_type = other DNA
caggtgcagctggtggagtctgggggaggcgtggtccagcctgggaggtccctgagactctcctgtgcagtctctggattcaccttcagtagctatggcatgcactgggtccgccaggctccaggcaaggggctggagtgggtggcagctatatcatatgatggaagtaataaatactatgtagactccgtgaagggccgattcaccatctccagagacaattccaagaaaacgctgtatctgcaaatgaacagcctgagagctgaggacacggctgtgtataattgtgcgaaaaatattgtactagtgatgtatgatatagactatcactactatgggatggacgtctggggccaagggaccacggtcaccgtctcctca

SEQ ID NO: 242 - protein, 127 aa, synthetic construct
  REGION 1..127: note = Synthetic
QVQLVESGGGVVQPGRSLRLSCAVSGFTFSSYGMHWVRQAPGKGLEWVAAISYDGSNKYYVDSVKGRFTISRDNSKKTLYLQMNSLRAEDTAVYNCAKNIVLVMYDIDYHYYGMDVWGQGTTVTVSS

SEQ ID NO: 243 - DNA, 24 bp, synthetic construct
  misc_feature 1..24: note = Synthetic
  source 1..24: mol_type = other DNA
ggattcaccttcagtagctatggc

SEQ ID NO: 244 - protein, 8 aa, synthetic construct
  REGION 1..8: note = Synthetic
GFTFSSYG

SEQ ID NO: 245 - DNA, 24 bp, synthetic construct
  misc_feature 1..24: note = Synthetic
  source 1..24: mol_type = other DNA
atatcatatgatggaagtaataaa

SEQ ID NO: 246 - protein, 8 aa, synthetic construct
  REGION 1..8: note = Synthetic
ISYDGSNK

SEQ ID NO: 247 - DNA, 60 bp, synthetic construct
  misc_feature 1..60: note = Synthetic
  source 1..60: mol_type = other DNA
gcgaaaaatattgtactagtgatgtatgatatagactatcactactatgggatggacgtc

SEQ ID NO: 248 - protein, 20 aa, synthetic construct
  REGION 1..20: note = Synthetic
AKNIVLVMYDIDYHYYGMDV

SEQ ID NO: 249 - DNA, 336 bp, synthetic construct
  misc_feature 1..336: note = Synthetic
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacaactcctgatctatttgggttttaatcgggcctccggggtccctgacaggttcagtggcagtggatcaggcacagattttacactgaaaatcagcagagtggaggctgaggatgttggggtttattactgcatgcaagctctacaaactcctctcactttcggcggagggaccaaggtggagatcaga

SEQ ID NO: 250 - protein, 112 aa, synthetic construct
  REGION 1..112: note = Synthetic
DIVMTQSPLSLPVTPGEPASISCRSSQSLLHSNGYNYLDWYLQKPGQSPQLLIYLGFNRASGVPDRFSGSGSGTDFTLKISRVEAEDVGVYYCMQALQTPLTFGGGTKVEIR

SEQ ID NO: 251 - DNA, 33 bp, synthetic construct
  misc_feature 1..33: note = Synthetic
  source 1..33: mol_type = other DNA
cagagcctcctgcatagtaatggatacaactat

SEQ ID NO: 252 - protein, 11 aa, synthetic construct
  REGION 1..11: note = Synthetic
QSLLHSNGYNY

SEQ ID NO: 253-254 - skipped (2 entries)

SEQ ID NO: 255 - DNA, 27 bp, synthetic construct
  misc_feature 1..27: note = Synthetic
  source 1..27: mol_type = other DNA
atgcaagctctacaaactcctctcact

SEQ ID NO: 256 - protein, 9 aa, synthetic construct
  REGION 1..9: note = Synthetic
MQALQTPLT

SEQ ID NO: 257 - DNA, 381 bp, synthetic construct; identical to SEQ ID NO: 241
  misc_feature 1..381: note = Synthetic
  source 1..381: mol_type = other DNA

SEQ ID NO: 258 - protein, 127 aa, synthetic construct; identical to SEQ ID NO: 242
  REGION 1..127: note = Synthetic

SEQ ID NO: 259 - DNA, 336 bp, synthetic construct
  misc_feature 1..336: note = Synthetic
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacaactcctgatctatttgggttttaatcgggcctccggggtccctgacaggttcagtggcagtggatcaggcacagattttacactgaaaatcagcagagtggaggctgaggatgttggggtttattactgcatgcaagctctacaaactcctctcactttcggcggagggaccaaggtggagatcaaa

SEQ ID NO: 260 - protein, 112 aa, synthetic construct
  REGION 1..112: note = Synthetic
DIVMTQSPLSLPVTPGEPASISCRSSQSLLHSNGYNYLDWYLQKPGQSPQLLIYLGFNRASGVPDRFSGSGSGTDFTLKISRVEAEDVGVYYCMQALQTPLTFGGGTKVEIK

SEQ ID NO: 261 - DNA, 381 bp, synthetic construct
  misc_feature 1..381: note = Synthetic
  source 1..381: mol_type = other DNA
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaaaatattgtactagtgatgtatgatatagactatcactactatgggatggacgtctgggggcaagggaccacggtcaccgtctcctca

SEQ ID NO: 262 - protein, 127 aa, synthetic construct
  REGION 1..127: note = Synthetic
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKNIVLVMYDIDYHYYGMDVWGQGTTVTVSS

SEQ ID NO: 263 - DNA, 336 bp, synthetic construct
  misc_feature 1..336: note = Synthetic
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttttaatcgggcctccggggtccctgacaggttcagtggcagtggatcaggcacagattttacactgaaaatcagcagagtggaggctgaggatgttggggtttattactgcatgcaagctctacaaactcctctcactttcggcggagggaccaaggtggagatcaaa

SEQ ID NO: 264 - protein, 112 aa, synthetic construct; identical to SEQ ID NO: 260
  REGION 1..112: note = Synthetic

SEQ ID NO: 265 - DNA, 381 bp, synthetic construct; identical to SEQ ID NO: 241
  misc_feature 1..381: note = Synthetic
  source 1..381: mol_type = other DNA

SEQ ID NO: 266 - protein, 127 aa, synthetic construct; identical to SEQ ID NO: 242
  REGION 1..127: note = Synthetic

SEQ ID NO: 267 - DNA, 24 bp, synthetic construct
  misc_feature 1..24: note = Synthetic
  source 1..24: mol_type = other DNA
ggattcaccttcagtagctatggc

SEQ ID NO: 268 - protein, 8 aa, synthetic construct
  REGION 1..8: note = Synthetic
GFTFSSYG

SEQ ID NO: 269 - DNA, 24 bp, synthetic construct
  misc_feature 1..24: note = Synthetic
  source 1..24: mol_type = other DNA
atatcatatgatggaagtaataaa

SEQ ID NO: 270 - protein, 8 aa, synthetic construct
  REGION 1..8: note = Synthetic
ISYDGSNK

SEQ ID NO: 271 - DNA, 60 bp, synthetic construct; identical to SEQ ID NO: 247
  misc_feature 1..60: note = Synthetic
  source 1..60: mol_type = other DNA

SEQ ID NO: 272 - protein, 20 aa, synthetic construct
  REGION 1..20: note = Synthetic
AKNIVLVMYDIDYHYYGMDV

SEQ ID NO: 273 - DNA, 336 bp, synthetic construct; identical to SEQ ID NO: 249
  misc_feature 1..336: note = Synthetic
  source 1..336: mol_type = other DNA

SEQ ID NO: 274 - protein, 112 aa, synthetic construct; identical to SEQ ID NO: 250
  REGION 1..112: note = Synthetic

SEQ ID NO: 275 - DNA, 33 bp, synthetic construct; identical to SEQ ID NO: 251
  misc_feature 1..33: note = Synthetic
  source 1..33: mol_type = other DNA

SEQ ID NO: 276 - protein, 11 aa, synthetic construct
  REGION 1..11: note = Synthetic
QSLLHSNGYNY

SEQ ID NO: 277-278 - skipped (2 entries)

SEQ ID NO: 279 - DNA, 27 bp, synthetic construct
  misc_feature 1..27: note = Synthetic
  source 1..27: mol_type = other DNA
atgcaagctctacaaactcctctcact

SEQ ID NO: 280 - protein, 9 aa, synthetic construct
  REGION 1..9: note = Synthetic
MQALQTPLT

SEQ ID NO: 281 - DNA, 381 bp, synthetic construct; identical to SEQ ID NO: 241
  misc_feature 1..381: note = Synthetic
  source 1..381: mol_type = other DNA

SEQ ID NO: 282 - protein, 127 aa, synthetic construct; identical to SEQ ID NO: 242
  REGION 1..127: note = Synthetic

SEQ ID NO: 283 - DNA, 336 bp, synthetic construct; identical to SEQ ID NO: 259
  misc_feature 1..336: note = Synthetic
  source 1..336: mol_type = other DNA

SEQ ID NO: 284 - protein, 112 aa, synthetic construct; identical to SEQ ID NO: 260
  REGION 1..112: note = Synthetic

SEQ ID NO: 285 - DNA, 381 bp, synthetic construct; identical to SEQ ID NO: 261
  misc_feature 1..381: note = Synthetic
  source 1..381: mol_type = other DNA

SEQ ID NO: 286 - protein, 127 aa, synthetic construct; identical to SEQ ID NO: 262
  REGION 1..127: note = Synthetic

SEQ ID NO: 287 - DNA, 336 bp, synthetic construct; identical to SEQ ID NO: 263
  misc_feature 1..336: note = Synthetic
  source 1..336: mol_type = other DNA

SEQ ID NO: 288 - protein, 112 aa, synthetic construct; identical to SEQ ID NO: 260
  REGION 1..112: note = Synthetic

SEQ ID NO: 289 - DNA, 372 bp, synthetic construct
  misc_feature 1..372: note = Synthetic
  source 1..372: mol_type = other DNA
cagatcaccttgaaggagtctggtcctacgctggtaaaacccacacagaccctcacgctgacctgcaccttctctgggttctcactcagcgctagtggagtgggtgtgggctggttccgtcagcccccaggaaaggccctggagtggcttgcactcatttattggaatgatgataagcgttacagcccatctctaaagaacagcctcaccatcaccaaggacacctccaaaaaccaggtggtccttacaatgaccaacatggaccctgtggacacagccacatattactgtgcacacagaatacatctatggtcctacttctactacggtatggacgtctggggccaagggaccacggtcaccgtctcctca

SEQ ID NO: 290 - protein, 124 aa, synthetic construct
  REGION 1..124: note = Synthetic
QITLKESGPTLVKPTQTLTLTCTFSGFSLSASGVGVGWFRQPPGKALEWLALIYWNDDKRYSPSLKNSLTITKDTSKNQVVLTMTNMDPVDTATYYCAHRIHLWSYFYYGMDVWGQGTTVTVSS

SEQ ID NO: 291 - DNA, 30 bp, synthetic construct
  misc_feature 1..30: note = Synthetic
  source 1..30: mol_type = other DNA
gggttctcactcagcgctagtggagtgggt

SEQ ID NO: 292 - protein, 10 aa, synthetic construct
  REGION 1..10: note = Synthetic
GFSLSASGVG

SEQ ID NO: 293 - DNA, 21 bp, synthetic construct
  misc_feature 1..21: note = Synthetic
  source 1..21: mol_type = other DNA
atttattggaatgatgataag

SEQ ID NO: 294 - protein, 7 aa, synthetic construct
  REGION 1..7: note = Synthetic
IYWNDDK

SEQ ID NO: 295 - DNA, 48 bp, synthetic construct
  misc_feature 1..48: note = Synthetic
  source 1..48: mol_type = other DNA
gcacacagaatacatctatggtcctacttctactacggtatggacgtc

SEQ ID NO: 296 - protein, 16 aa, synthetic construct
  REGION 1..16: note = Synthetic
AHRIHLWSYFYYGMDV

SEQ ID NO: 297 - DNA, 336 bp, synthetic construct
  misc_feature 1..336: note = Synthetic
  source 1..336: mol_type = other DNA
gatattgtgatgactcagtctccactctccctgcccgtcacccctggagagccggcctccatctcctgcaggtctagtcagactctcctgcatagtaatggatacaactatttcgattggtacctgcagaagccagggcagtctccacagctcctgatctatttgggttctaatcgggcctccggggtccctgacagattcagtggcagtggatcaggcacagattttacactgaaaatcagcagagtggaggctgaggatgttggaatttattactgcatgcaagctctacaaactcctctcactttcggcggagggaccaaggtggagatcaga

SEQ ID NO: 298 - protein, 112 aa, synthetic construct
  REGION 1..112: note = Synthetic
DIVMTQSPLSLPVTPGEPASISCRSSQTLLHSNGYNYFDWYLQKPGQSPQLLIYLGSNRASGVPDRFSGSGSGTDFTLKISRVEAEDVGIYYCMQALQTPLTFGGGTKVEIR

SEQ ID NO: 299 - DNA, 33 bp, synthetic construct
  misc_feature 1..33: note = Synthetic
  source 1..33: mol_type = other DNA
cagactctcctgcatagtaatggatacaactat

SEQ ID NO: 300 - protein, 11 aa, synthetic construct
  REGION 1..11: note = Synthetic
QTLLHSNGYNY

SEQ ID NO: 301-302 - skipped (2 entries)

SEQ ID NO: 303 - DNA, 27 bp, synthetic construct
  misc_feature 1..27: note = Synthetic
  source 1..27: mol_type = other DNA
atgcaagctctacaaactcctctcact

SEQ ID NO: 304 - protein, 9 aa, synthetic construct
  REGION 1..9: note = Synthetic
MQALQTPLT

SEQ ID NO: 305 - DNA, 372 bp, synthetic construct; identical to SEQ ID NO: 289
  misc_feature 1..372: note = Synthetic
  source 1..372: mol_type = other DNA

SEQ ID NO: 306 - protein, 124 aa, synthetic construct; identical to SEQ ID NO: 290
  REGION 1..124: note = Synthetic

SEQ ID NO: 307 - DNA, 336 bp, synthetic construct
  misc_feature 1..336: note = Synthetic
  source 1..336: mol_type = other DNA
gatattgtgatgactcagtctccactctccctgcccgtcacccctggagagccggcctccatctcctgcaggtctagtcagactctcctgcatagtaatggatacaactatttcgattggtacctgcagaagccagggcagtctccacagctcctgatctatttgggttctaatcgggcctccggggtccctgacagattcagtggcagtggatcaggcacagattttacactgaaaatcagcagagtggaggctgaggatgttggaatttattactgcatgcaagctctacaaactcctctcactttcggcggagggaccaaggtggagatcaaa

SEQ ID NO: 308 - protein, 112 aa, synthetic construct
  REGION 1..112: note = Synthetic
DIVMTQSPLSLPVTPGEPASISCRSSQTLLHSNGYNYFDWYLQKPGQSPQLLIYLGSNRASGVPDRFSGSGSGTDFTLKISRVEAEDVGIYYCMQALQTPLTFGGGTKVEIK

SEQ ID NO: 309 - DNA, 372 bp, synthetic construct
  misc_feature 1..372: note = Synthetic
  source 1..372: mol_type = other DNA
cagatcaccttgaaggagtctggtcctacgctggtgaaacccacacagaccctcacgctgacctgcaccttctctgggttctcactcagcgctagtggagtgggtgtgggctggatccgtcagcccccaggaaaggccctggagtggcttgcactcatttattggaatgatgataagcgctacagcccatctctgaagagcaggctcaccatcaccaaggacacctccaaaaaccaggtggtccttacaatgaccaacatggaccctgtggacacagccacatattactgtgcacacagaatacatctatggtcctacttctactacggtatggacgtctgggggcaagggaccacggtcaccgtctcctca

SEQ ID NO: 310 - protein, 124 aa, synthetic construct
  REGION 1..124: note = Synthetic
QITLKESGPTLVKPTQTLTLTCTFSGFSLSASGVGVGWIRQPPGKALEWLALIYWNDDKRYSPSLKSRLTITKDTSKNQVVLTMTNMDPVDTATYYCAHRIHLWSYFYYGMDVWGQGTTVTVSS

SEQ ID NO: 311 - DNA, 336 bp, synthetic construct
  misc_feature 1..336: note = Synthetic
  source 1..336: mol_type = other DNA
gatattgtgatgactcagtctccactctccctgcccgtcacccctggagagccggcctccatctcctgcaggtctagtcagactctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctctcactttcggcggagggaccaaggtggagatcaaa

SEQ ID NO: 312 - protein, 112 aa, synthetic construct
  REGION 1..112: note = Synthetic
DIVMTQSPLSLPVTPGEPASISCRSSQTLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 313 - DNA, 381 bp, synthetic construct
  misc_feature 1..381: note = Synthetic
  source 1..381: mol_type = other DNA
caggttcagctggtgcagtctggacctgaggtgaagaaccctggggcctcagtgaaggtctcctgcaaggcttctggttacacctttaccacctatggtatcagttgggtacgacaggcccctggacaagggcttgagtggatgggatggatcagcggttacaatggtaaaacaaacgatgcacagaagttccaggacagagtcgccatgaccacagacacatccacgagcacagcctacatggagctgaggagcctgagatctgacgacacggccatttattactgttcgagagatcgtttagtagtaccacctgcccttaattattcctactacgttatggacgtctggggccaagggaccacggtcaccgtctcctca

SEQ ID NO: 314 - protein, 127 aa, synthetic construct
  REGION 1..127: note = Synthetic
QVQLVQSGPEVKNPGASVKVSCKASGYTFTTYGISWVRQAPGQGLEWMGWISGYNGKTNDAQKFQDRVAMTTDTSTSTAYMELRSLRSDDTAIYYCSRDRLVVPPALNYSYYVMDVWGQGTTVTVSS

SEQ ID NO: 315 - DNA, 24 bp, synthetic construct
  misc_feature 1..24: note = Synthetic
  source 1..24: mol_type = other DNA
ggttacacctttaccacctatggt

SEQ ID NO: 316 - protein, 8 aa, synthetic construct
  REGION 1..8: note = Synthetic
GYTFTTYG

SEQ ID NO: 317 - DNA, 24 bp, synthetic construct
  misc_feature 1..24: note = Synthetic
  source 1..24: mol_type = other DNA
atcagcggttacaatggtaaaaca

SEQ ID NO: 318 - protein, 8 aa, synthetic construct
  REGION 1..8: note = Synthetic
ISGYNGKT

SEQ ID NO: 319 - DNA, 60 bp, synthetic construct
  misc_feature 1..60: note = Synthetic
  source 1..60: mol_type = other DNA
tcgagagatcgtttagtagtaccacctgcccttaattattcctactacgttatggacgtc

SEQ ID NO: 320 - protein, 20 aa, synthetic construct
  REGION 1..20: note = Synthetic
SRDRLVVPPALNYSYYVMDV

SEQ ID NO: 321 - DNA, 336 bp, synthetic construct
  misc_feature 1..336: note = Synthetic
  source 1..336: mol_type = other DNA
gatgttgtgatgactcagtctccactctccctgcccgtcacccttggacagccggcctccatctcctgcaggtctagtcaaagcctcgtatacagtgatggaaacacctacttgaattggtctcagcagaggccaggtcaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgtacacttttggccaggggaccaagctggagatcaaa

SEQ ID NO: 322 - protein, 112 aa, synthetic construct
  REGION 1..112: note = Synthetic
DVVMTQSPLSLPVTLGQPASISCRSSQSLVYSDGNTYLNWSQQRPGQSPRRLIYKVSNRDSGVPDRFSGSGSGTDFTLKISRVEAEDVGVYYCMQGTHWPYTFGQGTKLEIK

SEQ ID NO: 323 - DNA, 33 bp, synthetic construct
  misc_feature 1..33: note = Synthetic
  source 1..33: mol_type = other DNA
caaagcctcgtatacagtgatggaaacacctac

SEQ ID NO: 324 - protein, 11 aa, synthetic construct
  REGION 1..11: note = Synthetic
QSLVYSDGNTY

SEQ ID NO: 325-326 - skipped (2 entries)

SEQ ID NO: 327 - DNA, 27 bp, synthetic construct
  misc_feature 1..27: note = Synthetic
  source 1..27: mol_type = other DNA
atgcaaggtacacactggccgtacact

SEQ ID NO: 328 - protein, 9 aa, synthetic construct
  REGION 1..9: note = Synthetic
MQGTHWPYT

SEQ ID NO: 329 - DNA, 381 bp, synthetic construct; identical to SEQ ID NO: 313
  misc_feature 1..381: note = Synthetic
  source 1..381: mol_type = other DNA

SEQ ID NO: 330 - protein, 127 aa, synthetic construct; identical to SEQ ID NO: 314
  REGION 1..127: note = Synthetic

SEQ ID NO: 331 - DNA, 336 bp, synthetic construct; identical to SEQ ID NO: 321
  misc_feature 1..336: note = Synthetic
  source 1..336: mol_type = other DNA

SEQ ID NO: 332 - protein, 112 aa, synthetic construct; identical to SEQ ID NO: 322
  REGION 1..112: note = Synthetic

SEQ ID NO: 333 - DNA, 381 bp, synthetic construct
  misc_feature 1..381: note = Synthetic
  source 1..381: mol_type = other DNA
caggttcagctggtgcagtctggagctgaggtgaagaagcctggggcctcagtgaaggtctcctgcaaggcttctggttacacctttaccacctatggtatcagctgggtgcgacaggcccctggacaagggcttgagtggatgggatggatcagcggttacaatggtaaaacaaactatgcacagaagctccagggcagagtcaccatgaccacagacacatccacgagcacagcctacatggagctgaggagcctgagatctgacgacacggccgtgtattactgttcgagagatcgtttagtagtaccacctgcccttaattattcctactacgttatggacgtctgggggcaagggaccacggtcaccgtctcctca

SEQ ID NO: 334 - protein, 127 aa, synthetic construct
  REGION 1..127: note = Synthetic
QVQLVQSGAEVKKPGASVKVSCKASGYTFTTYGISWVRQAPGQGLEWMGWISGYNGKTNYAQKLQGRVTMTTDTSTSTAYMELRSLRSDDTAVYYCSRDRLVVPPALNYSYYVMDVWGQGTTVTVSS

SEQ ID NO: 335 - DNA, 336 bp, synthetic construct
  misc_feature 1..336: note = Synthetic
  source 1..336: mol_type = other DNA
gatgttgtgatgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgtacacttttggccaggggaccaagctggagatcaaa

SEQ ID NO: 336 - protein, 112 aa, synthetic construct
  REGION 1..112: note = Synthetic
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPYTFGQGTKLEIK

SEQ ID NO: 337 - DNA, 354 bp, synthetic construct
  misc_feature 1..354: note = Synthetic
  source 1..354: mol_type = other DNA
gaggtgcagctggtggagtctgggggaggcctggtcaagcctggggggtccctgagactctcctgtgcagcctctggattcaccttcagtagctatagcatggactgggtccgccaggctccagggaaggggctggagtgggtctcatccattagtagtagtagtagttacatatactacgcagactctgtgaagggccgattcaccatctccagagacaccgccaagaactcactgtatctgcaaatgaacagcctgagagacgaggacacggctgtttattactgtgcgagagagggcagtagcagactttttgactactggggccagggaaccctggtcaccgtctcctca

SEQ ID NO: 338 - protein, 118 aa, synthetic construct
  REGION 1..118: note = Synthetic
EVQLVESGGGLVKPGGSLRLSCAASGFTFSSYSMDWVRQAPGKGLEWVSSISSSSSYIYYADSVKGRFTISRDTAKNSLYLQMNSLRDEDTAVYYCAREGSSRLFDYWGQGTLVTVSS

SEQ ID NO: 339 - DNA, 24 bp, synthetic construct
  misc_feature 1..24: note = Synthetic
  source 1..24: mol_type = other DNA
ggattcaccttcagtagctatagc

SEQ ID NO: 340 - protein, 8 aa, synthetic construct
  REGION 1..8: note = Synthetic
GFTFSSYS

SEQ ID NO: 341 - DNA, 24 bp, synthetic construct
  misc_feature 1..24: note = Synthetic
  source 1..24: mol_type = other DNA
attagtagtagtagtagttacata

SEQ ID NO: 342 - protein, 8 aa, synthetic construct
  REGION 1..8: note = Synthetic
ISSSSSYI

SEQ ID NO: 343 - DNA, 33 bp, synthetic construct
  misc_feature 1..33: note = Synthetic
  source 1..33: mol_type = other DNA
gcgagagagggcagtagcagactttttgactac

SEQ ID NO: 344 - protein, 11 aa, synthetic construct
  REGION 1..11: note = Synthetic
AREGSSRLFDY

SEQ ID NO: 345 - DNA, 321 bp, synthetic construct
  misc_feature 1..321: note = Synthetic
  source 1..321: mol_type = other DNA
gacatccagatgacccagtctccttccaccctgtctgcatctgtaggagacagagtcaccatcacttgccgggccagtcagagtattagtagctggttggcctggtatcagcagagaccagggaaagcccctaagctcctgatctataaggcgtctagtttagaaggtggagtcccatcaaggttcagcggcagtggatctgggacagaattcactctcaccatcagcagcctgcagcctgaggattttgcaacttattactgccaacagtataatagttattggtacacttttggccaggggaccaagctggagatcaaa

SEQ ID NO: 346 - protein, 107 aa, synthetic construct
  REGION 1..107: note = Synthetic
DIQMTQSPSTLSASVGDRVTITCRASQSISSWLAWYQQRPGKAPKLLIYKASSLEGGVPSRFSGSGSGTEFTLTISSLQPEDFATYYCQQYNSYWYTFGQGTKLEIK

SEQ ID NO: 347 - DNA, 18 bp, synthetic construct
  misc_feature 1..18: note = Synthetic
  source 1..18: mol_type = other DNA
cagagtattagtagctgg

SEQ ID NO: 348 - protein, 6 aa, synthetic construct
  REGION 1..6: note = Synthetic
QSISSW

SEQ ID NO: 349-350 - skipped (2 entries)

SEQ ID NO: 351 - DNA, 27 bp, synthetic construct
  misc_feature 1..27: note = Synthetic
  source 1..27: mol_type = other DNA
caacagtataatagttattggtacact

SEQ ID NO: 352 - protein, 9 aa, synthetic construct
  REGION 1..9: note = Synthetic
QQYNSYWYT

SEQ ID NO: 353 - DNA, 354 bp, synthetic construct; identical to SEQ ID NO: 337
  misc_feature 1..354: note = Synthetic
  source 1..354: mol_type = other DNA

SEQ ID NO: 354 - protein, 118 aa, synthetic construct; identical to SEQ ID NO: 338
  REGION 1..118: note = Synthetic

SEQ ID NO: 355 - DNA, 321 bp, synthetic construct; identical to SEQ ID NO: 345
  misc_feature 1..321: note = Synthetic
  source 1..321: mol_type = other DNA

SEQ ID NO: 356 - protein, 107 aa, synthetic construct; identical to SEQ ID NO: 346
  REGION 1..107: note = Synthetic

SEQ ID NO: 357 - DNA, 354 bp, synthetic construct
  misc_feature 1..354: note = Synthetic
  source 1..354: mol_type = other DNA
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagagggcagtagcagactttttgactactggggccaaggaaccctggtcaccgtctcctca

SEQ ID NO: 358 - protein, 118 aa, synthetic construct
  REGION 1..118: note = Synthetic
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAREGSSRLFDYWGQGTLVTVSS

SEQ ID NO: 359 - DNA, 321 bp, synthetic construct
  misc_feature 1..321: note = Synthetic
  source 1..321: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattggtacacttttggccaggggaccaagctggagatcaaa

SEQ ID NO: 360 - protein, 107 aa, synthetic construct
  REGION 1..107: note = Synthetic
DIQMTQSPSTLSASVGDRVTITCRASQSISSWLAWYQQKPGKAPKLLIYKASSLESGVPSRFSGSGSGTEFTLTISSLQPDDFATYYCQQYNSYWYTFGQGTKLEIK

SEQ ID NO: 361 - DNA, 384 bp, synthetic construct
  misc_feature 1..384: note = Synthetic
  source 1..384: mol_type = other DNA
caggtgcacctggtggagtctgggggaggcttggtcaagcctggagggtccctgagactctcctgtgcagcctctggattcaccttcagtgaccactacatgagctggatccgccaggctccagggaaggggctggagtggatttcatacattagtaatgatggtggtaccaaatactatgtggactctgtggagggccgattcatcatttccagggacaacgccaagaactcattgtatctacatatgaacagcctcagagccgacgacacggccgtgtattactgtgcgagagatcagggatatattggctacgactcgtattattactattcctacggtatggacgtctggggccaagggaccacggtcaccgtcgcctca

SEQ ID NO: 362 - protein, 128 aa, synthetic construct
  REGION 1..128: note = Synthetic
QVHLVESGGGLVKPGGSLRLSCAASGFTFSDHYMSWIRQAPGKGLEWISYISNDGGTKYYVDSVEGRFIISRDNAKNSLYLHMNSLRADDTAVYYCARDQGYIGYDSYYYYSYGMDVWGQGTTVTVAS

SEQ ID NO: 363 - DNA, 24 bp, synthetic construct
  misc_feature 1..24: note = Synthetic
  source 1..24: mol_type = other DNA
ggattcaccttcagtgaccactac

SEQ ID NO: 364 - protein, 8 aa, synthetic construct
  REGION 1..8: note = Synthetic
GFTFSDHY

SEQ ID NO: 365 - DNA, 24 bp, synthetic construct
  misc_feature 1..24: note = Synthetic
  source 1..24: mol_type = other DNA
attagtaatgatggtggtaccaaa

SEQ ID NO: 366 - protein, 8 aa, synthetic construct
  REGION 1..8: note = Synthetic
ISNDGGTK

SEQ ID NO: 367 - DNA, 63 bp, synthetic construct
  misc_feature 1..63: note = Synthetic
  source 1..63: mol_type = other DNA
gcgagagatcagggatatattggctacgactcgtattattactattcctacggtatggacgtc

SEQ ID NO: 368 - protein, 21 aa, synthetic construct
  REGION 1..21: note = Synthetic
ARDQGYIGYDSYYYYSYGMDV

SEQ ID NO: 369 - DNA, 321 bp, synthetic construct
  misc_feature 1..321: note = Synthetic
  source 1..321: mol_type = other DNA
aaaattgtgttgacgcagtctccaggcaccctgcctttgtttccaggggaaagagccaccctctcctgtagggccagtcagagtgttaacaacaaattcttagcctggtaccagcagaaatctggccaggctcccaggctcctcatctatggtgcatccagcagggccactggcatcccagacaggttcagtggcagtgggtctgggaccgacttcactctcaccatcagcggactggagcctgaagattttgaagtgtattattgtcaagtatatggtaactcactcactctcggcggagggaccaaggtggagatcaag

SEQ ID NO: 370 - protein, 107 aa, synthetic construct
  REGION 1..107: note = Synthetic
KIVLTQSPGTLPLFPGERATLSCRASQSVNNKFLAWYQQKSGQAPRLLIYGASSRATGIPDRFSGSGSGTDFTLTISGLEPEDFEVYYCQVYGNSLTLGGGTKVEIK

SEQ ID NO: 371 - DNA, 21 bp, synthetic construct
  misc_feature 1..21: note = Synthetic
  source 1..21: mol_type = other DNA
cagagtgttaacaacaaattc

SEQ ID NO: 372 - protein, 7 aa, synthetic construct
  REGION 1..7: note = Synthetic
QSVNNKF

SEQ ID NO: 373-374 - skipped (2 entries)

SEQ ID NO: 375 - DNA, 24 bp, synthetic construct
  misc_feature 1..24: note = Synthetic
  source 1..24: mol_type = other DNA
caagtatatggtaactcactcact

SEQ ID NO: 376 - protein, 8 aa, synthetic construct
  REGION 1..8: note = Synthetic
QVYGNSLT

SEQ ID NO: 377 - DNA, 384 bp, synthetic construct
  misc_feature 1..384: note = Synthetic
  source 1..384: mol_type = other DNA
caggtgcagctggtggagtctgggggaggcttggtcaagcctggagggtccctgagactctcctgtgcagcctctggattcaccttcagtgaccactacatgagctggatccgccaggctccagggaaggggctggagtggatttcatacattagtaatgatggtggtaccaaatactatgtggactctgtggagggccgattcatcatttccagggacaacgccaagaactcattgtatctacatatgaacagcctcagagccgacgacacggccgtgtattactgtgcgagagatcagggatatattggctacgactcgtattattactattcctacggtatggacgtctggggccaagggaccacggtcaccgtctcctca

SEQ ID NO: 378 - protein, 128 aa, synthetic construct
  REGION 1..128: note = Synthetic
QVQLVESGGGLVKPGGSLRLSCAASGFTFSDHYMSWIRQAPGKGLEWISYISNDGGTKYYVDSVEGRFIISRDNAKNSLYLHMNSLRADDTAVYYCARDQGYIGYDSYYYYSYGMDVWGQGTTVTVSS

SEQ ID NO: 379 - DNA, 321 bp, synthetic construct
  misc_feature 1..321: note = Synthetic
  source 1..321: mol_type = other DNA
gaaattgtgttgacgcagtctccaggcaccctgcctttgtttccaggggaaagagccaccctctcctgtagggccagtcagagtgttaacaacaaattcttagcctggtaccagcagaaatctggccaggctcccaggctcctcatctatggtgcatccagcagggccactggcatcccagacaggttcagtggcagtgggtctgggaccgacttcactctcaccatcagcggactggagcctgaagattttgaagtgtattattgtcaagtatatggtaactcactcactctcggcggagggaccaaggtggagatcaaa

SEQ ID NO: 380 - protein, 107 aa, synthetic construct
  REGION 1..107: note = Synthetic
EIVLTQSPGTLPLFPGERATLSCRASQSVNNKFLAWYQQKSGQAPRLLIYGASSRATGIPDRFSGSGSGTDFTLTISGLEPEDFEVYYCQVYGNSLTLGGGTKVEIK

SEQ ID NO: 381 - DNA, 384 bp, synthetic construct
  misc_feature 1..384: note = Synthetic
  source 1..384: mol_type = other DNA
caggtgcagctggtggagtctgggggaggcttggtcaagcctggagggtccctgagactctcctgtgcagcctctggattcaccttcagtgaccactacatgagctggatccgccaggctccagggaaggggctggagtgggtttcatacattagtaatgatggtggtaccaaatactacgcagactctgtgaagggccgattcaccatctccagggacaacgccaagaactcactgtatctgcaaatgaacagcctgagagccgaggacacggccgtgtattactgtgcgagagatcagggatatattggctacgactcgtattattactattcctacggtatggacgtctgggggcaagggaccacggtcaccgtctcctca

SEQ ID NO: 382 - protein, 128 aa, synthetic construct
  REGION 1..128: note = Synthetic
QVQLVESGGGLVKPGGSLRLSCAASGFTFSDHYMSWIRQAPGKGLEWVSYISNDGGTKYYADSVKGRFTISRDNAKNSLYLQMNSLRAEDTAVYYCARDQGYIGYDSYYYYSYGMDVWGQGTTVTVSS

SEQ ID NO: 383 - DNA, 321 bp, synthetic construct
  misc_feature 1..321: note = Synthetic
  source 1..321: mol_type = other DNA
gaaattgtgttgacgcagtctccaggcaccctgtctttgtctccaggggaaagagccaccctctcctgcagggccagtcagagtgttaacaacaaattcttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcaagtatatggtaactcactcactttcggcggagggaccaaggtggagatcaaa

SEQ ID NO: 384 - protein, 107 aa, synthetic construct
  REGION 1..107: note = Synthetic
EIVLTQSPGTLSLSPGERATLSCRASQSVNNKFLAWYQQKPGQAPRLLIYGASSRATGIPDRFSGSGSGTDFTLTISRLEPEDFAVYYCQVYGNSLTFGGGTKVEIK

SEQ ID NO: 385 - DNA, 360 bp, synthetic construct
  misc_feature 1..360: note = Synthetic
  source 1..360: mol_type = other DNA
gaggtgcagaaggtggagtctgggggaggcctggtcaagccgggggggtccctgagactctcctgtacagcctctggattcaccttcagtacttataacatgaattgggtccgccaggctccagggaagggactggagtgggtctcatccattaggagtagtagtaattacatatactacgcagactcagtgaagggccgattcaccatctccagagacaacgccaagaattcactgtatctgcaaatgaacagcctgagagccgatgacacggctgtgtattactgtgcgagagatggcagcagttggtacgactactctgactactggggccagggaaccctggtcaccgtctcctca

SEQ ID NO: 386 - protein, 120 aa, synthetic construct
  REGION 1..120: note = Synthetic
EVQKVESGGGLVKPGGSLRLSCTASGFTFSTYNMNWVRQAPGKGLEWVSSIRSSSNYIYYADSVKGRFTISRDNAKNSLYLQMNSLRADDTAVYYCARDGSSWYDYSDYWGQGTLVTVSS

SEQ ID NO: 387 - DNA, 24 bp, synthetic construct
  misc_feature 1..24: note = Synthetic
  source 1..24: mol_type = other DNA
ggattcaccttcagtacttataac

SEQ ID NO: 388 - protein, 8 aa, synthetic construct
  REGION 1..8: note = Synthetic
GFTFSTYN

SEQ ID NO: 389 - DNA, 24 bp, synthetic construct
  misc_feature 1..24: note = Synthetic
  source 1..24: mol_type = other DNA
attaggagtagtagtaattacata

SEQ ID NO: 390 - protein, 8 aa, synthetic construct
  REGION 1..8: note = Synthetic
IRSSSNYI

SEQ ID NO: 391 - DNA, 39 bp, synthetic construct
  misc_feature 1..39: note = Synthetic
  source 1..39: mol_type = other DNA
gcgagagatggcagcagttggtacgactactctgactac

SEQ ID NO: 392 - protein, 13 aa, synthetic construct
  REGION 1..13: note = Synthetic
ARDGSSWYDYSDY

SEQ ID NO: 393 - DNA, 321 bp, synthetic construct
  misc_feature 1..321: note = Synthetic
  source 1..321: mol_type = other DNA
gacatccagatgacccagtctccttccaccctgtctgcatctgtaggagacagagtcaccatcacttgccgggccagtcagagtattagtagctggttggcctggtatcaacagataccagggaaagcccctaaactcctgatctataaggcgtctagtttagaaaatggggtcccatcaaggttcagcggcagtggatctgggacagaattcactctcatcatcagcagcctgcagcctgatgattttgcaacttattactgccaacagtatattagttattctcggacgttcggccaagggaccaaggtggaaatcaaa

SEQ ID NO: 394 - protein, 107 aa, synthetic construct
  REGION 1..107: note = Synthetic
DIQMTQSPSTLSASVGDRVTITCRASQSISSWLAWYQQIPGKAPKLLIYKASSLENGVPSRFSGSGSGTEFTLIISSLQPDDFATYYCQQYISYSRTFGQGTKVEIK

SEQ ID NO: 395 - DNA, 18 bp, synthetic construct
  misc_feature 1..18: note = Synthetic
  source 1..18: mol_type = other DNA
cagagtattagtagctgg

SEQ ID NO: 396 - protein, 6 aa, synthetic construct
  REGION 1..6: note = Synthetic
QSISSW

SEQ ID NO: 397-398 - skipped (2 entries)

SEQ ID NO: 399 - DNA, 27 bp, synthetic construct
  misc_feature 1..27: note = Synthetic
  source 1..27: mol_type = other DNA
caacagtatattagttattctcggacg

SEQ ID NO: 400 - protein, 9 aa, synthetic construct
  REGION 1..9: note = Synthetic
QQYISYSRT

SEQ ID NO: 401 - DNA, 360 bp, synthetic construct
  misc_feature 1..360: note = Synthetic
  source 1..360: mol_type = other DNA
gaggtgcagctggtggagtctgggggaggcctggtcaagccgggggggtccctgagactctcctgtacagcctctggattcaccttcagtacttataacatgaattgggtccgccaggctccagggaagggactggagtgggtctcatccattaggagtagtagtaattacatatactacgcagactcagtgaagggccgattcaccatctccagagacaacgccaagaattcactgtatctgcaaatgaacagcctgagagccgatgacacggctgtgtattactgtgcgagagatggcagcagttggtacgactactctgactactggggccagggaaccctggtcaccgtctcctca

SEQ ID NO: 402 - protein, 120 aa, synthetic construct
  REGION 1..120: note = Synthetic
EVQLVESGGGLVKPGGSLRLSCTASGFTFSTYNMNWVRQAPGKGLEWVSSIRSSSNYIYYADSVKGRFTISRDNAKNSLYLQMNSLRADDTAVYYCARDGSSWYDYSDYWGQGTLVTVSS

SEQ ID NO: 403 - DNA, 321 bp, synthetic construct; identical to SEQ ID NO: 393
  misc_feature 1..321: note = Synthetic
  source 1..321: mol_type = other DNA

SEQ ID NO: 404 - protein, 107 aa, synthetic construct; identical to SEQ ID NO: 394
  REGION 1..107: note = Synthetic

SEQ ID NO: 405 - DNA, 360 bp, synthetic construct
  misc_feature 1..360: note = Synthetic
  source 1..360: mol_type = other DNA
gaggtgcagctggtggagtctgggggaggcctggtcaagcctggggggtccctgagactctcctgtgcagcctctggattcaccttcagtacttataacatgaactgggtccgccaggctccagggaaggggctggagtgggtctcatccattaggagtagtagtaattacatatactacgcagactcagtgaagggccgattcaccatctccagagacaacgccaagaactcactgtatctgcaaatgaacagcctgagagccgaggacacggctgtgtattactgtgcgagagatggcagcagttggtacgactactctgactactggggccaaggaaccctggtcaccgtctcctca

SEQ ID NO: 406 - protein, 120 aa, synthetic construct
  REGION 1..120: note = Synthetic
EVQLVESGGGLVKPGGSLRLSCAASGFTFSTYNMNWVRQAPGKGLEWVSSIRSSSNYIYYADSVKGRFTISRDNAKNSLYLQMNSLRAEDTAVYYCARDGSSWYDYSDYWGQGTLVTVSS

SEQ ID NO: 407 - DNA, 321 bp, synthetic construct
  misc_feature 1..321: note = Synthetic
  source 1..321: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtatattagttattctcggacgttcggccaagggaccaaggtggaaatcaaa

SEQ ID NO: 408 - protein, 107 aa, synthetic construct
  REGION 1..107: note = Synthetic
DIQMTQSPSTLSASVGDRVTITCRASQSISSWLAWYQQKPGKAPKLLIYKASSLESGVPSRFSGSGSGTEFTLTISSLQPDDFATYYCQQYISYSRTFGQGTKVEIK

SEQ ID NO: 409 - DNA, 360 bp, synthetic construct; identical to SEQ ID NO: 401
  misc_feature 1..360: note = Synthetic
  source 1..360: mol_type = other DNA

SEQ ID NO: 410 - protein, 120 aa, synthetic construct; identical to SEQ ID NO: 402
  REGION 1..120: note = Synthetic

SEQ ID NO: 411 - DNA, 24 bp, synthetic construct
  misc_feature 1..24: note = Synthetic
  source 1..24: mol_type = other DNA
ggattcaccttcagtacttataac

SEQ ID NO: 412 - protein, 8 aa, synthetic construct
  REGION 1..8: note = Synthetic
GFTFSTYN

SEQ ID NO: 413 - DNA, 24 bp, synthetic construct
  misc_feature 1..24: note = Synthetic
  source 1..24: mol_type = other DNA
attaggagtagtagtaattacata

SEQ ID NO: 414 - protein, 8 aa, synthetic construct
  REGION 1..8: note = Synthetic
IRSSSNYI

SEQ ID NO: 415 - DNA, 39 bp, synthetic construct; identical to SEQ ID NO: 391
  misc_feature 1..39: note = Synthetic
  source 1..39: mol_type = other DNA

SEQ ID NO: 416 - protein, 13 aa, synthetic construct
  REGION 1..13: note = Synthetic
ARDGSSWYDYSDY

SEQ ID NO: 417 - DNA, 321 bp, synthetic construct; identical to SEQ ID NO: 393
  misc_feature 1..321: note = Synthetic
  source 1..321: mol_type = other DNA

SEQ ID NO: 418 - protein, 107 aa, synthetic construct; identical to SEQ ID NO: 394
  REGION 1..107: note = Synthetic

SEQ ID NO: 419 - DNA, 18 bp, synthetic construct
  misc_feature 1..18: note = Synthetic
  source 1..18: mol_type = other DNA
cagagtattagtagctgg

SEQ ID NO: 420 - protein, 6 aa, synthetic construct
  REGION 1..6: note = Synthetic
QSISSW

SEQ ID NO: 421-422 - skipped (2 entries)

SEQ ID NO: 423 - DNA, 27 bp, synthetic construct
  misc_feature 1..27: note = Synthetic
  source 1..27: mol_type = other DNA
caacagtatattagttattctcggacg

SEQ ID NO: 424 - protein, 9 aa, synthetic construct
  REGION 1..9: note = Synthetic
QQYISYSRT

SEQ ID NO: 425 - DNA, 360 bp, synthetic construct; identical to SEQ ID NO: 401
  misc_feature 1..360: note = Synthetic
  source 1..360: mol_type = other DNA

SEQ ID NO: 426 - protein, 120 aa, synthetic construct; identical to SEQ ID NO: 402
  REGION 1..120: note = Synthetic

SEQ ID NO: 427 - DNA, 321 bp, synthetic construct; identical to SEQ ID NO: 393
  misc_feature 1..321: note = Synthetic
  source 1..321: mol_type = other DNA

SEQ ID NO: 428 - protein, 107 aa, synthetic construct; identical to SEQ ID NO: 394
  REGION 1..107: note = Synthetic

SEQ ID NO: 429 - DNA, 360 bp, synthetic construct; identical to SEQ ID NO: 405
  misc_feature 1..360: note = Synthetic
  source 1..360: mol_type = other DNA

SEQ ID NO: 430 - protein, 120 aa, synthetic construct; identical to SEQ ID NO: 406
  REGION 1..120: note = Synthetic

SEQ ID NO: 431 - DNA, 321 bp, synthetic construct; identical to SEQ ID NO: 407
  misc_feature 1..321: note = Synthetic
  source 1..321: mol_type = other DNA

SEQ ID NO: 432 - protein, 107 aa, synthetic construct; identical to SEQ ID NO: 408
  REGION 1..107: note = Synthetic

SEQ ID NO: 433 - DNA, 360 bp, synthetic construct
  misc_feature 1..360: note = Synthetic
  source 1..360: mol_type = other DNA
gaggtgcagctggtggagtctgggggaggcctggtcaagccgggggggtccctgagactctcctgtacagcctctggattcaccttcagtacttataacatgaattgggtccgccaggctccagggaagggactggagtgggtctcatccattaggagtagtagtaattacatatactacgcagactcagtgaagggccgattcaccatctccagagacaacgccaagagttcactgtatctgcaaatgaacagcctgagagccgaggacacggctgtgtattactgtgcgagagatggcagcagttggtacgactactctgactactggggccagggaaccctggtcaccgtctcctca

SEQ ID NO: 434 - protein, 120 aa, synthetic construct
  REGION 1..120: note = Synthetic
EVQLVESGGGLVKPGGSLRLSCTASGFTFSTYNMNWVRQAPGKGLEWVSSIRSSSNYIYYADSVKGRFTISRDNAKSSLYLQMNSLRAEDTAVYYCARDGSSWYDYSDYWGQGTLVTVSS

SEQ ID NO: 435 - DNA, 24 bp, synthetic construct
  misc_feature 1..24: note = Synthetic
  source 1..24: mol_type = other DNA
ggattcaccttcagtacttataac

SEQ ID NO: 436 - protein, 8 aa, synthetic construct
  REGION 1..8: note = Synthetic
GFTFSTYN

SEQ ID NO: 437 - DNA, 24 bp, synthetic construct
  misc_feature 1..24: note = Synthetic
  source 1..24: mol_type = other DNA
attaggagtagtagtaattacata

SEQ ID NO: 438 - protein, 8 aa, synthetic construct
  REGION 1..8: note = Synthetic
IRSSSNYI

SEQ ID NO: 439 - DNA, 39 bp, synthetic construct; identical to SEQ ID NO: 391
  misc_feature 1..39: note = Synthetic
  source 1..39: mol_type = other DNA

SEQ ID NO: 440 - protein, 13 aa, synthetic construct
  REGION 1..13: note = Synthetic
ARDGSSWYDYSDY

SEQ ID NO: 441 - DNA, 321 bp, synthetic construct
  misc_feature 1..321: note = Synthetic
  source 1..321: mol_type = other DNA
gacatccagatgacccagtctccttccaccctgtctgcatctgtaggagacagagtcaccatcacttgccgggccagtcagagtattagtagctggttggcctggtatcaacaggtaccagggaaagcccctaaactcctgatctataaggcgtctagtttagaaaatggggtcccatcaaggttcagcggcagtggatctgggacagaattcactctcatcatcagcagcctgcagcctgatgattttgcaacttattactgccaacagtatattagttattctcggacgttcggccaagggaccaaggtggaaatcaaa

SEQ ID NO: 442 - protein, 107 aa, synthetic construct
  REGION 1..107: note = Synthetic
DIQMTQSPSTLSASVGDRVTITCRASQSISSWLAWYQQVPGKAPKLLIYKASSLENGVPSRFSGSGSGTEFTLIISSLQPDDFATYYCQQYISYSRTFGQGTKVEIK

SEQ ID NO: 443 - DNA, 18 bp, synthetic construct
  misc_feature 1..18: note = Synthetic
  source 1..18: mol_type = other DNA
cagagtattagtagctgg

SEQ ID NO: 444 - protein, 6 aa, synthetic construct
  REGION 1..6: note = Synthetic
QSISSW

SEQ ID NO: 445-446 - skipped (2 entries)

SEQ ID NO: 447 - DNA, 27 bp, synthetic construct
  misc_feature 1..27: note = Synthetic
  source 1..27: mol_type = other DNA
caacagtatattagttattctcggacg

SEQ ID NO: 448 - protein, 9 aa, synthetic construct
  REGION 1..9: note = Synthetic
QQYISYSRT

SEQ ID NO: 449 - DNA, 360 bp, synthetic construct; identical to SEQ ID NO: 433
  misc_feature 1..360: note = Synthetic
  source 1..360: mol_type = other DNA

SEQ ID NO: 450 - protein, 120 aa, synthetic construct; identical to SEQ ID NO: 434
  REGION 1..120: note = Synthetic

SEQ ID NO: 451 - DNA, 321 bp, synthetic construct; identical to SEQ ID NO: 441
  misc_feature 1..321: note = Synthetic
  source 1..321: mol_type = other DNA

SEQ ID NO: 452 - protein, 107 aa, synthetic construct; identical to SEQ ID NO: 442
  REGION 1..107: note = Synthetic

SEQ ID NO: 453 - DNA, 360 bp, synthetic construct
  misc_feature 1..360: note = Synthetic
  source 1..360: mol_type = other DNA
gaggtgcagctggtggagtctgggggaggcctggtcaagcctggggggtccctgagactctcctgtgcagcctctggattcaccttcagtacttataacatgaactgggtccgccaggctccagggaaggggctggagtgggtctcatccattaggagtagtagtaattacatatactacgcagactcagtgaagggccgattcaccatctccagagacaacgccaagaactcactgtatctgcaaatgaacagcctgagagccgaggacacggctgtgtattactgtgcgagagatggcagcagttggtacgactactctgactactggggccagggaaccctggtcaccgtctcctca

SEQ ID NO: 454 - protein, 120 aa, synthetic construct; identical to SEQ ID NO: 406
  REGION 1..120: note = Synthetic

SEQ ID NO: 455 - DNA, 321 bp, synthetic construct; identical to SEQ ID NO: 407
  misc_feature 1..321: note = Synthetic
  source 1..321: mol_type = other DNA

SEQ ID NO: 456 - protein, 107 aa, synthetic construct; identical to SEQ ID NO: 408
  REGION 1..107: note = Synthetic

SEQ ID NO: 457 - DNA, 360 bp, synthetic construct; identical to SEQ ID NO: 401
  misc_feature 1..360: note = Synthetic
  source 1..360: mol_type = other DNA

SEQ ID NO: 458 - protein, 120 aa, synthetic construct; identical to SEQ ID NO: 402
  REGION 1..120: note = Synthetic

SEQ ID NO: 459 - DNA, 24 bp, synthetic construct
  misc_feature 1..24: note = Synthetic
  source 1..24: mol_type = other DNA
ggattcaccttcagtacttataac

SEQ ID NO: 460 - protein, 8 aa, synthetic construct
  REGION 1..8: note = Synthetic
GFTFSTYN

SEQ ID NO: 461 - DNA, 24 bp, synthetic construct
  misc_feature 1..24: note = Synthetic
  source 1..24: mol_type = other DNA
attaggagtagtagtaattacata

SEQ ID NO: 462 - protein, 8 aa, synthetic construct
  REGION 1..8: note = Synthetic
IRSSSNYI

SEQ ID NO: 463 - DNA, 39 bp, synthetic construct; identical to SEQ ID NO: 391
  misc_feature 1..39: note = Synthetic
  source 1..39: mol_type = other DNA

SEQ ID NO: 464 - protein, 13 aa, synthetic construct
  REGION 1..13: note = Synthetic
ARDGSSWYDYSDY

SEQ ID NO: 465 - DNA, 321 bp, synthetic construct; identical to SEQ ID NO: 393
  misc_feature 1..321: note = Synthetic
  source 1..321: mol_type = other DNA

SEQ ID NO: 466 - protein, 107 aa, synthetic construct; identical to SEQ ID NO: 394
  REGION 1..107: note = Synthetic

SEQ ID NO: 467 - DNA, 18 bp, synthetic construct
  misc_feature 1..18: note = Synthetic
  source 1..18: mol_type = other DNA
cagagtattagtagctgg

SEQ ID NO: 468 - protein, 6 aa, synthetic construct
  REGION 1..6: note = Synthetic
QSISSW

SEQ ID NO: 469-470 - skipped (2 entries)

SEQ ID NO: 471 - DNA, 27 bp, synthetic construct
  misc_feature 1..27: note = Synthetic
  source 1..27: mol_type = other DNA
caacagtatattagttattctcggacg

SEQ ID NO: 472 - protein, 9 aa, synthetic construct
  REGION 1..9: note = Synthetic
QQYISYSRT

SEQ ID NO: 473 - DNA, 360 bp, synthetic construct; identical to SEQ ID NO: 401
  misc_feature 1..360: note = Synthetic
  source 1..360: mol_type = other DNA

SEQ ID NO: 474 - protein, 120 aa, synthetic construct; identical to SEQ ID NO: 402
  REGION 1..120: note = Synthetic

SEQ ID NO: 475 - DNA, 321 bp, synthetic construct; identical to SEQ ID NO: 393
  misc_feature 1..321: note = Synthetic
  source 1..321: mol_type = other DNA

SEQ ID NO: 476 - protein, 107 aa, synthetic construct; identical to SEQ ID NO: 394
  REGION 1..107: note = Synthetic

SEQ ID NO: 477 - DNA, 360 bp, synthetic construct; identical to SEQ ID NO: 405
  misc_feature 1..360: note = Synthetic
  source 1..360: mol_type = other DNA

SEQ ID NO: 478 - protein, 120 aa, synthetic construct; identical to SEQ ID NO: 406
  REGION 1..120: note = Synthetic

SEQ ID NO: 479 - DNA, 321 bp, synthetic construct; identical to SEQ ID NO: 407
  misc_feature 1..321: note = Synthetic
  source 1..321: mol_type = other DNA

SEQ ID NO: 480 - protein, 107 aa, synthetic construct; identical to SEQ ID NO: 408
  REGION 1..107: note = Synthetic

SEQ ID NO: 481 - DNA, 354 bp, synthetic construct
  misc_feature 1..354: note = Synthetic
  source 1..354: mol_type = other DNA
gaggtgcaactagtggagtctgggggaggcttggtacagcctggggggtccctgagactctcctgtgtagtctctggattcaccttcggtgactacgacatgcactgggtccgtcaagctacaggaagaggtctggagtgggtctcaggtattgctcctgctggtgacacatcctatacaggctccgtgaagggccgattcaccatctccagagagaatgccaagaactccttgcatcttcaaatgaacagcctgacaaccggggacacggctatatattattgtgctagagaggatatagcagtgcctggttttgattactggggccagggaaccctggtcaccgtctcctca

SEQ ID NO: 482 - protein, 118 aa, synthetic construct
  REGION 1..118: note = Synthetic
EVQLVESGGGLVQPGGSLRLSCVVSGFTFGDYDMHWVRQATGRGLEWVSGIAPAGDTSYTGSVKGRFTISRENAKNSLHLQMNSLTTGDTAIYYCAREDIAVPGFDYWGQGTLVTVSS

SEQ ID NO: 483 - DNA, 24 bp, synthetic construct
  misc_feature 1..24: note = Synthetic
  source 1..24: mol_type = other DNA
ggattcaccttcggtgactacgac

SEQ ID NO: 484 - protein, 8 aa, synthetic construct
  REGION 1..8: note = Synthetic
GFTFGDYD

SEQ ID NO: 485 - DNA, 21 bp, synthetic construct
  misc_feature 1..21: note = Synthetic
  source 1..21: mol_type = other DNA
attgctcctgctggtgacaca

SEQ ID NO: 486 - protein, 7 aa, synthetic construct
  REGION 1..7: note = Synthetic
IAPAGDT

SEQ ID NO: 487 - DNA, 36 bp, synthetic construct
  misc_feature 1..36: note = Synthetic
  source 1..36: mol_type = other DNA
gctagagaggatatagcagtgcctggttttgattac

SEQ ID NO: 488 - protein, 12 aa, synthetic construct
  REGION 1..12: note = Synthetic
AREDIAVPGFDY

SEQ ID NO: 489 - DNA, 324 bp, synthetic construct
  misc_feature 1..324: note = Synthetic
  source 1..324: mol_type = other DNA
gaaatagtgatgacgcagtctccagccaccctgtctgtgtctccaggggaacgaggcaccctctcctgcagggccagtcagagtgttagcagcaacttagcctggtaccagcagaaacctggccaggctcccagactcctcatctatggtgcatccacgagggccactggcttcccagccaggttcagtggcagtgggtctgggacagagttcactctcaccatcagcagcctgcagtctgaagattttgcagtttattactgtcagcagtataataagtggcctccgttcactttcggccctgggaccaaagtggatttcaaa

SEQ ID NO: 490 - protein, 108 aa, synthetic construct
  REGION 1..108: note = Synthetic
EIVMTQSPATLSVSPGERGTLSCRASQSVSSNLAWYQQKPGQAPRLLIYGASTRATGFPARFSGSGSGTEFTLTISSLQSEDFAVYYCQQYNKWPPFTFGPGTKVDFK

SEQ ID NO: 491 - DNA, 18 bp, synthetic construct
  misc_feature 1..18: note = Synthetic
  source 1..18: mol_type = other DNA
cagagtgttagcagcaac

SEQ ID NO: 492 - protein, 6 aa, synthetic construct
  REGION 1..6: note = Synthetic
QSVSSN

SEQ ID NO: 493-494 - skipped (2 entries)

SEQ ID NO: 495 - DNA, 30 bp, synthetic construct
  misc_feature 1..30: note = Synthetic
  source 1..30: mol_type = other DNA
cagcagtataataagtggcctccgttcact

SEQ ID NO: 496 - protein, 10 aa, synthetic construct
  REGION 1..10: note = Synthetic
QQYNKWPPFT

SEQ ID NO: 497 - DNA, 354 bp, synthetic construct; identical to SEQ ID NO: 481
  misc_feature 1..354: note = Synthetic
  source 1..354: mol_type = other DNA

SEQ ID NO: 498 - protein, 118 aa, synthetic construct; identical to SEQ ID NO: 482
  REGION 1..118: note = Synthetic

SEQ ID NO: 499 - DNA, 324 bp, synthetic construct
  misc_feature 1..324: note = Synthetic
  source 1..324: mol_type = other DNA
gaaatagtgatgacgcagtctccagccaccctgtctgtgtctccaggggaacgaggcaccctctcctgcagggccagtcagagtgttagcagcaacttagcctggtaccagcagaaacctggccaggctcccagactcctcatctatggtgcatccacgagggccactggcttcccagccaggttcagtggcagtgggtctgggacagagttcactctcaccatcagcagcctgcagtctgaagattttgcagtttattactgtcagcagtataataagtggcctccgttcactttcggccctgggaccaaagtggatatcaaa

SEQ ID NO: 500 - protein, 108 aa, synthetic construct
  REGION 1..108: note = Synthetic
EIVMTQSPATLSVSPGERGTLSCRASQSVSSNLAWYQQKPGQAPRLLIYGASTRATGFPARFSGSGSGTEFTLTISSLQSEDFAVYYCQQYNKWPPFTFGPGTKVDIK

SEQ ID NO: 501 - DNA, 354 bp, synthetic construct
  misc_feature 1..354: note = Synthetic
  source 1..354: mol_type = other DNA
gaggtgcagctggtggagtctgggggaggcttggtacagcctggggggtccctgagactctcctgtgcagcctctggattcaccttcggtgactacgacatgcactgggtccgccaagctacaggaaaaggtctggagtgggtctcagctattgctcctgctggtgacacatactatccaggctccgtgaagggccgattcaccatctccagagaaaatgccaagaactccttgtatcttcaaatgaacagcctgagagccggggacacggctgtgtattactgtgctagagaggatatagcagtgcctggttttgattactggggccaaggaaccctggtcaccgtctcctca

SEQ ID NO: 502 - protein, 118 aa, synthetic construct
  REGION 1..118: note = Synthetic
EVQLVESGGGLVQPGGSLRLSCAASGFTFGDYDMHWVRQATGKGLEWVSAIAPAGDTYYPGSVKGRFTISRENAKNSLYLQMNSLRAGDTAVYYCAREDIAVPGFDYWGQGTLVTVSS

SEQ ID NO: 503 - DNA, 324 bp, synthetic construct
  misc_feature 1..324: note = Synthetic
  source 1..324: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataagtggcctccgttcactttcggccctgggaccaaagtggatatcaaa

SEQ ID NO: 504 - protein, 108 aa, synthetic construct
  REGION 1..108: note = Synthetic
EIVMTQSPATLSVSPGERATLSCRASQSVSSNLAWYQQKPGQAPRLLIYGASTRATGIPARFSGSGSGTEFTLTISSLQSEDFAVYYCQQYNKWPPFTFGPGTKVDIK

SEQ ID NO: 505 - DNA, 378 bp, synthetic construct
  misc_feature 1..378: note = Synthetic
  source 1..378: mol_type = other DNA
caaattctgctggtgcaatctggacctgaggtgaaggagcctggggcctcagtgaaggtctcctgcaaggcttctggttacacctttaccaactacgctatcagctgggtgcgacaggtccctggacaagggcttgagtggatgggatgggtcagcgcttacaatggtcacacaaactatgcacatgaagtccagggcagagtcaccatgaccacagacacatccacgaccacagcctacatggagctgaggagcctgagatctgacgacacggccatgtattactgtgcgagagggggtgtagtcgtgccagttgctccccacttctacaacggtatggacgtctggggccaagggaccacggtcaccgtctcctca

SEQ ID NO: 506 - protein, 126 aa, synthetic construct
  REGION 1..126: note = Synthetic
QILLVQSGPEVKEPGASVKVSCKASGYTFTNYAISWVRQVPGQGLEWMGWVSAYNGHTNYAHEVQGRVTMTTDTSTTTAYMELRSLRSDDTAMYYCARGGVVVPVAPHFYNGMDVWGQGTTVTVSS

SEQ ID NO: 507 - DNA, 24 bp, synthetic construct
  misc_feature 1..24: note = Synthetic
  source 1..24: mol_type = other DNA
ggttacacctttaccaactacgct

SEQ ID NO: 508 - protein, 8 aa, synthetic construct
  REGION 1..8: note = Synthetic
GYTFTNYA

SEQ ID NO: 509 - DNA, 24 bp, synthetic construct
  misc_feature 1..24: note = Synthetic
  source 1..24: mol_type = other DNA
gtcagcgcttacaatggtcacaca

SEQ ID NO: 510 - protein, 8 aa, synthetic construct
  REGION 1..8: note = Synthetic
VSAYNGHT

SEQ ID NO: 511 - DNA, 57 bp, synthetic construct
  misc_feature 1..57: note = Synthetic
  source 1..57: mol_type = other DNA
gcgagagggggtgtagtcgtgccagttgctccccacttctacaacggtatggacgtc

SEQ ID NO: 512 - protein, 19 aa, synthetic construct
  REGION 1..19: note = Synthetic
ARGGVVVPVAPHFYNGMDV

SEQ ID NO: 513 - DNA, 336 bp, synthetic construct
  misc_feature 1..336: note = Synthetic
  source 1..336: mol_type = other DNA
gatattgtgatgactcagtttccactctccctgcccgtcacccctggagagccggcctccatctcctgcaggtctagtcagagcctcctgcatattaatgaatacaactatttggattggtacctaaagaagccagggcagtctccacagctcctgatctatttgggttttaatcgggcctccggggtccctgacaggttcagtggcagtggatcaggcacagattttacactgaaaatcagcagagtggaggctgaggatgttggggtctattactgcatgcaagctcttcaaactccgtggacgttaggccaagggaccaaggtggaaatcaaa

SEQ ID NO: 514 - protein, 112 aa, synthetic construct
  REGION 1..112: note = Synthetic
DIVMTQFPLSLPVTPGEPASISCRSSQSLLHINEYNYLDWYLKKPGQSPQLLIYLGFNRASGVPDRFSGSGSGTDFTLKISRVEAEDVGVYYCMQALQTPWTLGQGTKVEIK

SEQ ID NO: 515 - DNA, 33 bp, synthetic construct
  misc_feature 1..33: note = Synthetic
  source 1..33: mol_type = other DNA
cagagcctcctgcatattaatgaatacaactat

SEQ ID NO: 516 - protein, 11 aa, synthetic construct
  REGION 1..11: note = Synthetic
QSLLHINEYNY

SEQ ID NO: 517-518 - skipped (2 entries)

SEQ ID NO: 519 - DNA, 27 bp, synthetic construct
  misc_feature 1..27: note = Synthetic
  source 1..27: mol_type = other DNA
atgcaagctcttcaaactccgtggacg

SEQ ID NO: 520 - protein, 9 aa, synthetic construct
  REGION 1..9: note = Synthetic
MQALQTPWT

SEQ ID NO: 521 - DNA, 378 bp, synthetic construct
  misc_feature 1..378: note = Synthetic
  source 1..378: mol_type = other DNA
caggttcagctggtgcagtctggacctgaggtgaaggagcctggggcctcagtgaaggtctcctgcaaggcttctggttacacctttaccaactacgctatcagctgggtgcgacaggtccctggacaagggcttgagtggatgggatgggtcagcgcttacaatggtcacacaaactatgcacatgaagtccagggcagagtcaccatgaccacagacacatccacgaccacagcctacatggagctgaggagcctgagatctgacgacacggccatgtattactgtgcgagagggggtgtagtcgtgccagttgctccccacttctacaacggtatggacgtctggggccaagggaccacggtcaccgtctcctca

SEQ ID NO: 522 - protein, 126 aa, synthetic construct
  REGION 1..126: note = Synthetic
QVQLVQSGPEVKEPGASVKVSCKASGYTFTNYAISWVRQVPGQGLEWMGWVSAYNGHTNYAHEVQGRVTMTTDTSTTTAYMELRSLRSDDTAMYYCARGGVVVPVAPHFYNGMDVWGQGTTVTVSS

SEQ ID NO: 523 - DNA, 336 bp, synthetic construct
  misc_feature 1..336: note = Synthetic
  source 1..336: mol_type = other DNA
gatattgtgatgactcagtctccactctccctgcccgtcacccctggagagccggcctccatctcctgcaggtctagtcagagcctcctgcatattaatgaatacaactatttggattggtacctaaagaagccagggcagtctccacagctcctgatctatttgggttttaatcgggcctccggggtccctgacaggttcagtggcagtggatcaggcacagattttacactgaaaatcagcagagtggaggctgaggatgttggggtctattactgcatgcaagctcttcaaactccgtggacgttaggccaagggaccaaggtggaaatcaaa

SEQ ID NO: 524 - protein, 112 aa, synthetic construct
  REGION 1..112: note = Synthetic
DIVMTQSPLSLPVTPGEPASISCRSSQSLLHINEYNYLDWYLKKPGQSPQLLIYLGFNRASGVPDRFSGSGSGTDFTLKISRVEAEDVGVYYCMQALQTPWTLGQGTKVEIK

SEQ ID NO: 525 - DNA, 378 bp, synthetic construct
  misc_feature 1..378: note = Synthetic
  source 1..378: mol_type = other DNA
caggttcagctggtgcagtctggagctgaggtgaagaagcctggggcctcagtgaaggtctcctgcaaggcttctggttacacctttaccaactacgctatcagctgggtgcgacaggcccctggacaagggcttgagtggatgggatgggtcagcgcttacaatggtcacacaaactatgcacagaagctccagggcagagtcaccatgaccacagacacatccacgagcacagcctacatggagctgaggagcctgagatctgacgacacggccgtgtattactgtgcgagagggggtgtagtcgtgccagttgctccccacttctacaacggtatggacgtctgggggcaagggaccacggtcaccgtctcctca

SEQ ID NO: 526 - protein, 126 aa, synthetic construct
  REGION 1..126: note = Synthetic
QVQLVQSGAEVKKPGASVKVSCKASGYTFTNYAISWVRQAPGQGLEWMGWVSAYNGHTNYAQKLQGRVTMTTDTSTSTAYMELRSLRSDDTAVYYCARGGVVVPVAPHFYNGMDVWGQGTTVTVSS

SEQ ID NO: 527 - DNA, 336 bp, synthetic construct
  misc_feature 1..336: note = Synthetic
  source 1..336: mol_type = other DNA
gatattgtgatgactcagtctccactctccctgcccgtcacccctggagagccggcctccatctcctgcaggtctagtcagagcctcctgcatattaatgaatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctcttcaaactccgtggacgttcggccaagggaccaaggtggaaatcaaa

SEQ ID NO: 528 - protein, 112 aa, synthetic construct
  REGION 1..112: note = Synthetic
DIVMTQSPLSLPVTPGEPASISCRSSQSLLHINEYNYLDWYLQKPGQSPQLLIYLGSNRASGVPDRFSGSGSGTDFTLKISRVEAEDVGVYYCMQALQTPWTFGQGTKVEIK

SEQ ID NO: 529 - DNA, 351 bp, synthetic construct
  misc_feature 1..351: note = Synthetic
  source 1..351: mol_type = other DNA
gaggtgcagctggtggagtctgggggaggcttggtacagcctggggggtccctgagactctcctgtgcagcctctggattcaccctaagtagctacgacatgcactgggtccgccaagcaacaggaaaaggtctggagtgggtctcagctattggcagtactggtgacacatactatacaggctccgtgatgggccgattcaccatctccagagacgctgccaaaaactccttctatcttgaaatgaacagcctgagagtcggggacacggctgtatattactgtgcaagagagggaataagaacaccctatgattattggggccagggagcccgggtcaccgtctcctca

SEQ ID NO: 530 - protein, 117 aa, synthetic construct
  REGION 1..117: note = Synthetic
EVQLVESGGGLVQPGGSLRLSCAASGFTLSSYDMHWVRQATGKGLEWVSAIGSTGDTYYTGSVMGRFTISRDAAKNSFYLEMNSLRVGDTAVYYCAREGIRTPYDYWGQGARVTVSS

SEQ ID NO: 531 - DNA, 24 bp, synthetic construct
  misc_feature 1..24: note = Synthetic
  source 1..24: mol_type = other DNA
ggattcaccctaagtagctacgac

SEQ ID NO: 532 - protein, 8 aa, synthetic construct
  REGION 1..8: note = Synthetic
GFTLSSYD

SEQ ID NO: 533 - DNA, 21 bp, synthetic construct
  misc_feature 1..21: note = Synthetic
  source 1..21: mol_type = other DNA
attggcagtactggtgacaca

SEQ ID NO: 534 - protein, 7 aa, synthetic construct
  REGION 1..7: note = Synthetic
IGSTGDT

SEQ ID NO: 535 - DNA, 33 bp, synthetic construct
  misc_feature 1..33: note = Synthetic
  source 1..33: mol_type = other DNA
gcaagagagggaataagaacaccctatgattat

SEQ ID NO: 536 - protein, 11 aa, synthetic construct
  REGION 1..11: note = Synthetic
AREGIRTPYDY

SEQ ID NO: 537 - DNA, 324 bp, synthetic construct
  misc_feature 1..324: note = Synthetic
  source 1..324: mol_type = other DNA
gaaatagtgatgacgcagtctccagccaccctgtctgtgtctccaggggaaagagccaccctctcctgcagggccagtcagagtgttagcagcaatgtagcctggtaccagcagaaacctggccaggctcccaggctcctcatctatggtgcatccaccagggccactggtatcccagccaggttcagtggcagtgggtctgggacagaattcactctcaccatcagcagcctgcagtctgaagattttgcagtttattactgtcagcagtataataattggcctccattcactttcggccctgggaccaaagtggatatcaaa

SEQ ID NO: 538 - protein, 108 aa, synthetic construct
  REGION 1..108: note = Synthetic
EIVMTQSPATLSVSPGERATLSCRASQSVSSNVAWYQQKPGQAPRLLIYGASTRATGIPARFSGSGSGTEFTLTISSLQSEDFAVYYCQQYNNWPPFTFGPGTKVDIK

SEQ ID NO: 539 - DNA, 18 bp, synthetic construct
  misc_feature 1..18: note = Synthetic
  source 1..18: mol_type = other DNA
cagagtgttagcagcaat

SEQ ID NO: 540 - protein, 6 aa, synthetic construct
  REGION 1..6: note = Synthetic
QSVSSN

SEQ ID NO: 541-542 - skipped (2 entries)

SEQ ID NO: 543 - DNA, 30 bp, synthetic construct
  misc_feature 1..30: note = Synthetic
  source 1..30: mol_type = other DNA
cagcagtataataattggcctccattcact

SEQ ID NO: 544 - protein, 10 aa, synthetic construct
  REGION 1..10: note = Synthetic
QQYNNWPPFT

SEQ ID NO: 545 - DNA, 351 bp, synthetic construct
  misc_feature 1..351: note = Synthetic
  source 1..351: mol_type = other DNA
gaggtgcagctggtggagtctgggggaggcttggtacagcctggggggtccctgagactctcctgtgcagcctctggattcaccctaagtagctacgacatgcactgggtccgccaagcaacaggaaaaggtctggagtgggtctcagctattggcagtactggtgacacatactatacaggctccgtgatgggccgattcaccatctccagagacgctgccaaaaactccttctatcttgaaatgaacagcctgagagtcggggacacggctgtatattactgtgcaagagagggaataagaacaccctatgattattggggccagggaaccctggtcaccgtctcctca

SEQ ID NO: 546 - protein, 117 aa, synthetic construct
  REGION 1..117: note = Synthetic
EVQLVESGGGLVQPGGSLRLSCAASGFTLSSYDMHWVRQATGKGLEWVSAIGSTGDTYYTGSVMGRFTISRDAAKNSFYLEMNSLRVGDTAVYYCAREGIRTPYDYWGQGTLVTVSS

SEQ ID NO: 547 - DNA, 324 bp, synthetic construct; identical to SEQ ID NO: 537
  misc_feature 1..324: note = Synthetic
  source 1..324: mol_type = other DNA

SEQ ID NO: 548 - protein, 108 aa, synthetic construct; identical to SEQ ID NO: 538
  REGION 1..108: note = Synthetic

SEQ ID NO: 549 - DNA, 351 bp, synthetic construct
  misc_feature 1..351: note = Synthetic
  source 1..351: mol_type = other DNA
gaggtgcagctggtggagtctgggggaggcttggtacagcctggggggtccctgagactctcctgtgcagcctctggattcaccctaagtagctacgacatgcactgggtccgccaagctacaggaaaaggtctggagtgggtctcagctattggcagtactggtgacacatactatccaggctccgtgaagggccgattcaccatctccagagaaaatgccaagaactccttgtatcttcaaatgaacagcctgagagccggggacacggctgtgtattactgtgcaagagagggaataagaacaccctatgattattggggccaaggaaccctggtcaccgtctcctca

SEQ ID NO: 550 - protein, 117 aa, synthetic construct
  REGION 1..117: note = Synthetic
EVQLVESGGGLVQPGGSLRLSCAASGFTLSSYDMHWVRQATGKGLEWVSAIGSTGDTYYPGSVKGRFTISRENAKNSLYLQMNSLRAGDTAVYYCAREGIRTPYDYWGQGTLVTVSS

SEQ ID NO: 551 - DNA, 324 bp, synthetic construct
  misc_feature 1..324: note = Synthetic
  source 1..324: mol_type = other DNA
gaaatagtgatgacgcagtctccagccaccctgtctgtgtctccaggggaaagagccaccctctcctgcagggccagtcagagtgttagcagcaatttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataattggcctccattcactttcggccctgggaccaaagtggatatcaaa

SEQ ID NO: 552 - protein, 108 aa, synthetic construct; identical to SEQ ID NO: 24
  REGION 1..108: note = Synthetic

SEQ ID NO: 553 - DNA, 351 bp, synthetic construct; identical to SEQ ID NO: 529
  misc_feature 1..351: note = Synthetic
  source 1..351: mol_type = other DNA

SEQ ID NO: 554 - protein, 117 aa, synthetic construct; identical to SEQ ID NO: 530
  REGION 1..117: note = Synthetic

SEQ ID NO: 555 - DNA, 24 bp, synthetic construct
  misc_feature 1..24: note = Synthetic
  source 1..24: mol_type = other DNA
ggattcaccctaagtagctacgac

SEQ ID NO: 556 - protein, 8 aa, synthetic construct
  REGION 1..8: note = Synthetic
GFTLSSYD

SEQ ID NO: 557 - DNA, 21 bp, synthetic construct
  misc_feature 1..21: note = Synthetic
  source 1..21: mol_type = other DNA
attggcagtactggtgacaca

SEQ ID NO: 558 - protein, 7 aa, synthetic construct
  REGION 1..7: note = Synthetic
IGSTGDT

SEQ ID NO: 559 - DNA, 33 bp, synthetic construct; identical to SEQ ID NO: 535
  misc_feature 1..33: note = Synthetic
  source 1..33: mol_type = other DNA

SEQ ID NO: 560 - protein, 11 aa, synthetic construct
  REGION 1..11: note = Synthetic
AREGIRTPYDY

SEQ ID NO: 561 - DNA, 324 bp, synthetic construct; identical to SEQ ID NO: 537
  misc_feature 1..324: note = Synthetic
  source 1..324: mol_type = other DNA

SEQ ID NO: 562 - protein, 108 aa, synthetic construct; identical to SEQ ID NO: 538
  REGION 1..108: note = Synthetic

SEQ ID NO: 563 - DNA, 18 bp, synthetic construct
  misc_feature 1..18: note = Synthetic
  source 1..18: mol_type = other DNA
cagagtgttagcagcaat

SEQ ID NO: 564 - protein, 6 aa, synthetic construct
  REGION 1..6: note = Synthetic
QSVSSN

SEQ ID NO: 565-566 - skipped (2 entries)

SEQ ID NO: 567 - DNA, 30 bp, synthetic construct; identical to SEQ ID NO: 543
  misc_feature 1..30: note = Synthetic
  source 1..30: mol_type = other DNA

SEQ ID NO: 568 - protein, 10 aa, synthetic construct
  REGION 1..10: note = Synthetic
QQYNNWPPFT

SEQ ID NO: 569 - DNA, 351 bp, synthetic construct; identical to SEQ ID NO: 545
  misc_feature 1..351: note = Synthetic
  source 1..351: mol_type = other DNA

SEQ ID NO: 570 - protein, 117 aa, synthetic construct; identical to SEQ ID NO: 546
  REGION 1..117: note = Synthetic

SEQ ID NO: 571 - DNA, 324 bp, synthetic construct; identical to SEQ ID NO: 537
  misc_feature 1..324: note = Synthetic
  source 1..324: mol_type = other DNA

SEQ ID NO: 572 - protein, 108 aa, synthetic construct; identical to SEQ ID NO: 538
  REGION 1..108: note = Synthetic

SEQ ID NO: 573 - DNA, 351 bp, synthetic construct; identical to SEQ ID NO: 549
  misc_feature 1..351: note = Synthetic
  source 1..351: mol_type = other DNA

SEQ ID NO: 574 - protein, 117 aa, synthetic construct; identical to SEQ ID NO: 550
  REGION 1..117: note = Synthetic

SEQ ID NO: 575 - DNA, 324 bp, synthetic construct; identical to SEQ ID NO: 551
  misc_feature 1..324: note = Synthetic
  source 1..324: mol_type = other DNA

SEQ ID NO: 576 - protein, 108 aa, synthetic construct; identical to SEQ ID NO: 24
  REGION 1..108: note = Synthetic

SEQ ID NO: 577 - DNA, 363 bp, synthetic construct
  misc_feature 1..363: note = Synthetic
  source 1..363: mol_type = other DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattaattggaacagtggtagcataggctatgcggactctgtgaagggccgattcaccatctccagagacaacgccaagcactccctgtatctgcaaatgaacagtctgagacctgaggacacggccttgtattactgtgtaaaagaggtgactacgggatactactacggtatggacgtctggggccaagggaccacggtcaccgtctcctca

SEQ ID NO: 578 - protein, 121 aa, synthetic construct
  REGION 1..121: note = Synthetic
EVQLVESGGGLVQPGRSLRLSCAASGFTFDDYAMHWVRQAPGKGLEWVSGINWNSGSIGYADSVKGRFTISRDNAKHSLYLQMNSLRPEDTALYYCVKEVTTGYYYGMDVWGQGTTVTVSS

SEQ ID NO: 579 - DNA, 24 bp, synthetic construct
  misc_feature 1..24: note = Synthetic
  source 1..24: mol_type = other DNA
ggattcacctttgatgattatgcc

SEQ ID NO: 580 - protein, 8 aa, synthetic construct
  REGION 1..8: note = Synthetic
GFTFDDYA

SEQ ID NO: 581 - DNA, 24 bp, synthetic construct
  misc_feature 1..24: note = Synthetic
  source 1..24: mol_type = other DNA
attaattggaacagtggtagcata

SEQ ID NO: 582 - protein, 8 aa, synthetic construct
  REGION 1..8: note = Synthetic
INWNSGSI

SEQ ID NO: 583 - DNA, 42 bp, synthetic construct
  misc_feature 1..42: note = Synthetic
  source 1..42: mol_type = other DNA
gtaaaagaggtgactacgggatactactacggtatggacgtc

SEQ ID NO: 584 - protein, 14 aa, synthetic construct
  REGION 1..14: note = Synthetic
VKEVTTGYYYGMDV

SEQ ID NO: 585 - DNA, 321 bp, synthetic construct
  misc_feature 1..321: note = Synthetic
  source 1..321: mol_type = other DNA
gacatccagttgacccagtctccatccttcctgtctgcatctgtaggagacagagtcaccatcacttgctgggccagtcagggcattagcagttatttagcctggtatcagaaaaaaccagggaaagcccctaacctcctgatctatgatgcatccactttgcaaagtggggtcccatcaaggttcagcggcagtggatctgggacagaattcactctcacactcagcagcctgcagcctgaagattttgcaacttattactgtcaacagcttaatatttacccattcactttcggccctgggaccaaagtggatatcaaa

SEQ ID NO: 586 - protein, 107 aa, synthetic construct
  REGION 1..107: note = Synthetic
DIQLTQSPSFLSASVGDRVTITCWASQGISSYLAWYQKKPGKAPNLLIYDASTLQSGVPSRFSGSGSGTEFTLTLSSLQPEDFATYYCQQLNIYPFTFGPGTKVDIK

SEQ ID NO: 587 - DNA, 18 bp, synthetic construct
  misc_feature 1..18: note = Synthetic
  source 1..18: mol_type = other DNA
cagggcattagcagttat

SEQ ID NO: 588 - protein, 6 aa, synthetic construct
  REGION 1..6: note = Synthetic
QGISSY

SEQ ID NO: 589-590 - skipped (2 entries)

SEQ ID NO: 591 - DNA, 27 bp, synthetic construct
  misc_feature 1..27: note = Synthetic
  source 1..27: mol_type = other DNA
caacagcttaatatttacccattcact

SEQ ID NO: 592 - protein, 9 aa, synthetic construct
  REGION 1..9: note = Synthetic
QQLNIYPFT

SEQ ID NO: 593 - DNA, 363 bp, synthetic construct; identical to SEQ ID NO: 577
  misc_feature 1..363: note = Synthetic
  source 1..363: mol_type = other DNA

SEQ ID NO: 594 - protein, 121 aa, synthetic construct; identical to SEQ ID NO: 578
  REGION 1..121: note = Synthetic

SEQ ID NO: 595 - DNA, 321 bp, synthetic construct; identical to SEQ ID NO: 585
  misc_feature 1..321: note = Synthetic
  source 1..321: mol_type = other DNA

SEQ ID NO: 596 - protein, 107 aa, synthetic construct; identical to SEQ ID NO: 586
  REGION 1..107: note = Synthetic

SEQ ID NO: 597 - DNA, 363 bp, synthetic construct
  misc_feature 1..363: note = Synthetic
  source 1..363: mol_type = other DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattaattggaacagtggtagcataggctatgcggactctgtgaagggccgattcaccatctccagagacaacgccaagaactccctgtatctgcaaatgaacagtctgagagctgaggacacggccttgtattactgtgtaaaagaggtgactacgggatactactacggtatggacgtctgggggcaagggaccacggtcaccgtctcctca

SEQ ID NO: 598 - protein, 121 aa, synthetic construct
  REGION 1..121: note = Synthetic
EVQLVESGGGLVQPGRSLRLSCAASGFTFDDYAMHWVRQAPGKGLEWVSGINWNSGSIGYADSVKGRFTISRDNAKNSLYLQMNSLRAEDTALYYCVKEVTTGYYYGMDVWGQGTTVTVSS

SEQ ID NO: 599 - DNA, 321 bp, synthetic construct
  misc_feature 1..321: note = Synthetic
  source 1..321: mol_type = other DNA
gacatccagttgacccagtctccatccttcctgtctgcatctgtaggagacagagtcaccatcacttgccgggccagtcagggcattagcagttatttagcctggtatcagcaaaaaccagggaaagcccctaagctcctgatctatgatgcatccactttgcaaagtggggtcccatcaaggttcagcggcagtggatctgggacagaattcactctcacaatcagcagcctgcagcctgaagattttgcaacttattactgtcaacagcttaatatttacccattcactttcggccctgggaccaaagtggatatcaaa

SEQ ID NO: 600 - protein, 107 aa, synthetic construct
  REGION 1..107: note = Synthetic
DIQLTQSPSFLSASVGDRVTITCRASQGISSYLAWYQQKPGKAPKLLIYDASTLQSGVPSRFSGSGSGTEFTLTISSLQPEDFATYYCQQLNIYPFTFGPGTKVDIK

SEQ ID NO: 601 - DNA, 366 bp, synthetic construct
  misc_feature 1..366: note = Synthetic
  source 1..366: mol_type = other DNA
gaggtgcagttgttggagtctgggggaggcttggtacagcctggggggtccctgagactctcctgtgcagcctctggattcacgtttagtagctatgccatgaactgggtccgccaggctccagggaaggggctggattgggtctcaggtatcagtggtaatggtggtagcacctactacgcagactccgtgaagggccggttcaccatctccagagacatttccaagaacacgctgtatgtgcaaatgcacagcctgagagtcgaggacacggccgtttactactgtgcgaaagcccgttattacgatttttggggggggaatttcgatctctggggccgtggcacccaggtcactgtctcctca

SEQ ID NO: 602 - protein, 122 aa, synthetic construct
  REGION 1..122: note = Synthetic
EVQLLESGGGLVQPGGSLRLSCAASGFTFSSYAMNWVRQAPGKGLDWVSGISGNGGSTYYADSVKGRFTISRDISKNTLYVQMHSLRVEDTAVYYCAKARYYDFWGGNFDLWGRGTQVTVSS

SEQ ID NO: 603 - DNA, 24 bp, synthetic construct
  misc_feature 1..24: note = Synthetic
  source 1..24: mol_type = other DNA
ggattcacgtttagtagctatgcc

SEQ ID NO: 604 - protein, 8 aa, synthetic construct
  REGION 1..8: note = Synthetic
GFTFSSYA

SEQ ID NO: 605 - DNA, 24 bp, synthetic construct
  misc_feature 1..24: note = Synthetic
  source 1..24: mol_type = other DNA
atcagtggtaatggtggtagcacc

SEQ ID NO: 606 - protein, 8 aa, synthetic construct
  REGION 1..8: note = Synthetic
ISGNGGST

SEQ ID NO: 607 - DNA, 45 bp, synthetic construct
  misc_feature 1..45: note = Synthetic
  source 1..45: mol_type = other DNA
gcgaaagcccgttattacgatttttggggggggaatttcgatctc

SEQ ID NO: 608 - protein, 15 aa, synthetic construct
  REGION 1..15: note = Synthetic
AKARYYDFWGGNFDL

SEQ ID NO: 609 - DNA, 324 bp, synthetic construct
  misc_feature 1..324: note = Synthetic
  source 1..324: mol_type = other DNA
gaaattgtgttgacgcagtctccaggcaccctgtctttgtctccaggggaaagagccaccctctcctgcagggccagtcagagtgttagcatcaggtacttagcctggtatcagcagaaacctggccaggctcccaggctcctcatctatggtgcatccagcagggccactggcatcccagacaggttcagtgtcagtgtgtctgggacagacttcactctcaccatcactagactggagcctgaagattttgcagtctattactgtcagcaatatggtagttcaccgctcactttcggcggagggaccaaggtggagatcaaa

SEQ ID NO: 610 - protein, 108 aa, synthetic construct
  REGION 1..108: note = Synthetic
EIVLTQSPGTLSLSPGERATLSCRASQSVSIRYLAWYQQKPGQAPRLLIYGASSRATGIPDRFSVSVSGTDFTLTITRLEPEDFAVYYCQQYGSSPLTFGGGTKVEIK

SEQ ID NO: 611 - DNA, 21 bp, synthetic construct
  misc_feature 1..21: note = Synthetic
  source 1..21: mol_type = other DNA
cagagtgttagcatcaggtac

SEQ ID NO: 612 - protein, 7 aa, synthetic construct
  REGION 1..7: note = Synthetic
QSVSIRY

SEQ ID NO: 613-614 - skipped (2 entries)

SEQ ID NO: 615 - DNA, 27 bp, synthetic construct
  misc_feature 1..27: note = Synthetic
  source 1..27: mol_type = other DNA
cagcaatatggtagttcaccgctcact

SEQ ID NO: 616 - protein, 9 aa, synthetic construct
  REGION 1..9: note = Synthetic
QQYGSSPLT

SEQ ID NO: 617 - DNA, 366 bp, synthetic construct
  misc_feature 1..366: note = Synthetic
  source 1..366: mol_type = other DNA
gaggtgcagttgttggagtctgggggaggcttggtacagcctggggggtccctgagactctcctgtgcagcctctggattcacgtttagtagctatgccatgaactgggtccgccaggctccagggaaggggctggattgggtctcaggtatcagtggtaatggtggtagcacctactacgcagactccgtgaagggccggttcaccatctccagagacatttccaagaacacgctgtatgtgcaaatgcacagcctgagagtcgaggacacggccgtttactactgtgcgaaagcccgttattacgatttttggggggggaatttcgatctctggggccgtggcaccctggtcactgtctcctca

SEQ ID NO: 618 - protein, 122 aa, synthetic construct
  REGION 1..122: note = Synthetic
EVQLLESGGGLVQPGGSLRLSCAASGFTFSSYAMNWVRQAPGKGLDWVSGISGNGGSTYYADSVKGRFTISRDISKNTLYVQMHSLRVEDTAVYYCAKARYYDFWGGNFDLWGRGTLVTVSS

SEQ ID NO: 619 - DNA, 324 bp, synthetic construct; identical to SEQ ID NO: 609
  misc_feature 1..324: note = Synthetic
  source 1..324: mol_type = other DNA

SEQ ID NO: 620 - protein, 108 aa, synthetic construct; identical to SEQ ID NO: 610
  REGION 1..108: note = Synthetic

SEQ ID NO: 621 - DNA, 366 bp, synthetic construct
  misc_feature 1..366: note = Synthetic
  source 1..366: mol_type = other DNA
gaggtgcagctgttggagtctgggggaggcttggtacagcctggggggtccctgagactctcctgtgcagcctctggattcacgtttagtagctatgccatgagctgggtccgccaggctccagggaaggggctggagtgggtctcagctatcagtggtaatggtggtagcacctactacgcagactccgtgaagggccggttcaccatctccagagacaattccaagaacacgctgtatctgcaaatgaacagcctgagagccgaggacacggccgtatattactgtgcgaaagcccgttattacgatttttggggggggaatttcgatctctggggccgtggcaccctggtcactgtctcctca

SEQ ID NO: 622 - protein, 122 aa, synthetic construct
  REGION 1..122: note = Synthetic
EVQLLESGGGLVQPGGSLRLSCAASGFTFSSYAMSWVRQAPGKGLEWVSAISGNGGSTYYADSVKGRFTISRDNSKNTLYLQMNSLRAEDTAVYYCAKARYYDFWGGNFDLWGRGTLVTVSS

SEQ ID NO: 623 - DNA, 324 bp, synthetic construct
  misc_feature 1..324: note = Synthetic
  source 1..324: mol_type = other DNA
gaaattgtgttgacgcagtctccaggcaccctgtctttgtctccaggggaaagagccaccctctcctgcagggccagtcagagtgttagcatcaggtacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcaatatggtagttcaccgctcactttcggcggagggaccaaggtggagatcaaa

SEQ ID NO: 624 - protein, 108 aa, synthetic construct
  REGION 1..108: note = Synthetic
EIVLTQSPGTLSLSPGERATLSCRASQSVSIRYLAWYQQKPGQAPRLLIYGASSRATGIPDRFSGSGSGTDFTLTISRLEPEDFAVYYCQQYGSSPLTFGGGTKVEIK

SEQ ID NO: 625 - DNA, 381 bp, synthetic construct
  misc_feature 1..381: note = Synthetic
  source 1..381: mol_type = other DNA
caggttcagctggtgcagtctggacctgaggtgaagaaccctggggcctcagtgaaggtctcctgcaaggcttctggttacacctttaccacctatggtatcagttgggtacgacaggcccctggacaagggcttgagtggatgggatggatcagcggttacaatggtaaaacaaacgatgcacagaagttccaggacagagtcgccatgaccacagacacatccacgagcacagcctacatggagctgaggagcctgagatctgacgacacggccatttattactgttcgagagatcgtttagtagtaccacctgccctttattattcctactacgttatggacgtctggggccaagggaccacggtcaccgtctcctca

SEQ ID NO: 626 - protein, 127 aa, synthetic construct
  REGION 1..127: note = Synthetic
QVQLVQSGPEVKNPGASVKVSCKASGYTFTTYGISWVRQAPGQGLEWMGWISGYNGKTNDAQKFQDRVAMTTDTSTSTAYMELRSLRSDDTAIYYCSRDRLVVPPALYYSYYVMDVWGQGTTVTVSS

SEQ ID NO: 627 - DNA, 24 bp, synthetic construct
  misc_feature 1..24: note = Synthetic
  source 1..24: mol_type = other DNA
ggttacacctttaccacctatggt

SEQ ID NO: 628 - protein, 8 aa, synthetic construct
  REGION 1..8: note = Synthetic
GYTFTTYG

SEQ ID NO: 629 - DNA, 24 bp, synthetic construct
  misc_feature 1..24: note = Synthetic
  source 1..24: mol_type = other DNA
atcagcggttacaatggtaaaaca

SEQ ID NO: 630 - protein, 8 aa, synthetic construct
  REGION 1..8: note = Synthetic
ISGYNGKT

SEQ ID NO: 631 - DNA, 60 bp, synthetic construct
  misc_feature 1..60: note = Synthetic
  source 1..60: mol_type = other DNA
tcgagagatcgtttagtagtaccacctgccctttattattcctactacgttatggacgtc

SEQ ID NO: 632 - protein, 20 aa, synthetic construct
  REGION 1..20: note = Synthetic
SRDRLVVPPALYYSYYVMDV

SEQ ID NO: 633 - DNA, 336 bp, synthetic construct
  misc_feature 1..336: note = Synthetic
  source 1..336: mol_type = other DNA
gatgttgtgatgactcagtctccactctccctgcccgtcacccttggacagccggcctccatctcctgcaggtctagtcaaagcctcgtatacagtgatggaaacacctacttgaattggtttcagcagaggccaggtcaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgtacacttttggccaggggaccaagctggagatcaaa

SEQ ID NO: 634 - protein, 112 aa, synthetic construct; identical to SEQ ID NO: 336
  REGION 1..112: note = Synthetic

SEQ ID NO: 635 - DNA, 33 bp, synthetic construct; identical to SEQ ID NO: 323
  misc_feature 1..33: note = Synthetic
  source 1..33: mol_type = other DNA

SEQ ID NO: 636 - protein, 11 aa, synthetic construct
  REGION 1..11: note = Synthetic
QSLVYSDGNTY

SEQ ID NO: 637-638 - skipped (2 entries)

SEQ ID NO: 639 - DNA, 27 bp, synthetic construct
  misc_feature 1..27: note = Synthetic
  source 1..27: mol_type = other DNA
atgcaaggtacacactggccgtacact

SEQ ID NO: 640 - protein, 9 aa, synthetic construct
  REGION 1..9: note = Synthetic
MQGTHWPYT

SEQ ID NO: 641 - DNA, 381 bp, synthetic construct; identical to SEQ ID NO: 625
  misc_feature 1..381: note = Synthetic
  source 1..381: mol_type = other DNA

SEQ ID NO: 642 - protein, 127 aa, synthetic construct; identical to SEQ ID NO: 626
  REGION 1..127: note = Synthetic

SEQ ID NO: 643 - DNA, 336 bp, synthetic construct; identical to SEQ ID NO: 633
  misc_feature 1..336: note = Synthetic
  source 1..336: mol_type = other DNA

SEQ ID NO: 644 - protein, 112 aa, synthetic construct; identical to SEQ ID NO: 336
  REGION 1..112: note = Synthetic

SEQ ID NO: 645 - DNA, 381 bp, synthetic construct
  misc_feature 1..381: note = Synthetic
  source 1..381: mol_type = other DNA
caggttcagctggtgcagtctggagctgaggtgaagaagcctggggcctcagtgaaggtctcctgcaaggcttctggttacacctttaccacctatggtatcagctgggtgcgacaggcccctggacaagggcttgagtggatgggatggatcagcggttacaatggtaaaacaaactatgcacagaagctccagggcagagtcaccatgaccacagacacatccacgagcacagcctacatggagctgaggagcctgagatctgacgacacggccgtgtattactgttcgagagatcgtttagtagtaccacctgccctttattattcctactacgttatggacgtctgggggcaagggaccacggtcaccgtctcctca

SEQ ID NO: 646 - protein, 127 aa, synthetic construct
  REGION 1..127: note = Synthetic
QVQLVQSGAEVKKPGASVKVSCKASGYTFTTYGISWVRQAPGQGLEWMGWISGYNGKTNYAQKLQGRVTMTTDTSTSTAYMELRSLRSDDTAVYYCSRDRLVVPPALYYSYYVMDVWGQGTTVTVSS

SEQ ID NO: 647 - DNA, 336 bp, synthetic construct; identical to SEQ ID NO: 335
  misc_feature 1..336: note = Synthetic
  source 1..336: mol_type = other DNA

SEQ ID NO: 648 - protein, 112 aa, synthetic construct; identical to SEQ ID NO: 336
  REGION 1..112: note = Synthetic

SEQ ID NO: 649 - DNA, 381 bp, synthetic construct
  misc_feature 1..381: note = Synthetic
  source 1..381: mol_type = other DNA
caggttcagctggtgcagtctggacctgaggtgaagaaccctggggcctcagtgaaggtctcctgcaaggcttctggttacacctttaccacctatggtatcagttgggtacgacaggcccctggacaagggcttgagtggatgggatggatcagcggttacaatggtaaaacaaacgatgcacagaagttccaggacagagtcgccatgaccacagacacatccacgagcacagcctacatggagctgaggagcctgagatctgacgacacggccatttattactgttcgagagatcgtttagtagtaccacctgcccttaattattactactacgttatggacgtctggggccaagggaccacggtcaccgtctcctca

SEQ ID NO: 650 - protein, 127 aa, synthetic construct
  REGION 1..127: note = Synthetic
QVQLVQSGPEVKNPGASVKVSCKASGYTFTTYGISWVRQAPGQGLEWMGWISGYNGKTNDAQKFQDRVAMTTDTSTSTAYMELRSLRSDDTAIYYCSRDRLVVPPALNYYYYVMDVWGQGTTVTVSS

SEQ ID NO: 651 - DNA, 24 bp, synthetic construct
  misc_feature 1..24: note = Synthetic
  source 1..24: mol_type = other DNA
ggttacacctttaccacctatggt

SEQ ID NO: 652 - protein, 8 aa, synthetic construct
  REGION 1..8: note = Synthetic
GYTFTTYG

SEQ ID NO: 653 - DNA, 24 bp, synthetic construct
  misc_feature 1..24: note = Synthetic
  source 1..24: mol_type = other DNA
atcagcggttacaatggtaaaaca

SEQ ID NO: 654 - protein, 8 aa, synthetic construct
  REGION 1..8: note = Synthetic
ISGYNGKT

SEQ ID NO: 655 - DNA, 60 bp, synthetic construct
  misc_feature 1..60: note = Synthetic
  source 1..60: mol_type = other DNA
tcgagagatcgtttagtagtaccacctgcccttaattattactactacgttatggacgtc

SEQ ID NO: 656 - protein, 20 aa, synthetic construct
  REGION 1..20: note = Synthetic
SRDRLVVPPALNYYYYVMDV

SEQ ID NO: 657 - DNA, 336 bp, synthetic construct; identical to SEQ ID NO: 633
  misc_feature 1..336: note = Synthetic
  source 1..336: mol_type = other DNA

SEQ ID NO: 658 - protein, 112 aa, synthetic construct; identical to SEQ ID NO: 336
  REGION 1..112: note = Synthetic

SEQ ID NO: 659 - DNA, 33 bp, synthetic construct; identical to SEQ ID NO: 323
  misc_feature 1..33: note = Synthetic
  source 1..33: mol_type = other DNA

SEQ ID NO: 660 - protein, 11 aa, synthetic construct
  REGION 1..11: note = Synthetic
QSLVYSDGNTY

SEQ ID NO: 661-662 - skipped (2 entries)

SEQ ID NO: 663 - DNA, 27 bp, synthetic construct
  misc_feature 1..27: note = Synthetic
  source 1..27: mol_type = other DNA
atgcaaggtacacactggccgtacact

SEQ ID NO: 664 - protein, 9 aa, synthetic construct
  REGION 1..9: note = Synthetic
MQGTHWPYT

SEQ ID NO: 665 - DNA, 381 bp, synthetic construct; identical to SEQ ID NO: 649
  misc_feature 1..381: note = Synthetic
  source 1..381: mol_type = other DNA

SEQ ID NO: 666 - protein, 127 aa, synthetic construct; identical to SEQ ID NO: 650
  REGION 1..127: note = Synthetic

SEQ ID NO: 667 - DNA, 336 bp, synthetic construct; identical to SEQ ID NO: 633
  misc_feature 1..336: note = Synthetic
  source 1..336: mol_type = other DNA

SEQ ID NO: 668 - protein, 112 aa, synthetic construct; identical to SEQ ID NO: 336
  REGION 1..112: note = Synthetic

SEQ ID NO: 669 - DNA, 381 bp, synthetic construct
  misc_feature 1..381: note = Synthetic
  source 1..381: mol_type = other DNA
caggttcagctggtgcagtctggagctgaggtgaagaagcctggggcctcagtgaaggtctcctgcaaggcttctggttacacctttaccacctatggtatcagctgggtgcgacaggcccctggacaagggcttgagtggatgggatggatcagcggttacaatggtaaaacaaactatgcacagaagctccagggcagagtcaccatgaccacagacacatccacgagcacagcctacatggagctgaggagcctgagatctgacgacacggccgtgtattactgttcgagagatcgtttagtagtaccacctgcccttaattattactactacgttatggacgtctgggggcaagggaccacggtcaccgtctcctca

SEQ ID NO: 670 - protein, 127 aa, synthetic construct
  REGION 1..127: note = Synthetic
QVQLVQSGAEVKKPGASVKVSCKASGYTFTTYGISWVRQAPGQGLEWMGWISGYNGKTNYAQKLQGRVTMTTDTSTSTAYMELRSLRSDDTAVYYCSRDRLVVPPALNYYYYVMDVWGQGTTVTVSS

SEQ ID NO: 671 - DNA, 336 bp, synthetic construct; identical to SEQ ID NO: 335
  misc_feature 1..336: note = Synthetic
  source 1..336: mol_type = other DNA

SEQ ID NO: 672 - protein, 112 aa, synthetic construct; identical to SEQ ID NO: 336
  REGION 1..112: note = Synthetic

SEQ ID NO: 673 - DNA, 381 bp, synthetic construct
  misc_feature 1..381: note = Synthetic
  source 1..381: mol_type = other DNA
caggttcagctggtgcagtctggacctgaggtgaagaaccctggggcctcagtgaaggtctcctgcaaggcttctggttacacctttaccacctatggtatcagttgggtacgacaggcccctggacaagggcttgagtggatgggatggatcagcggttacaatggtaaaacaaacgatgcacagaagttccaggacagagtcgccatgaccacagacacatccacgagcacagcctacatggagctgaggagcctgagatctgacgacacggccatttattactgttcgagagatcgtttagtagtaccacctgccctttattattactactacgttatggacgtctggggccaagggaccacggtcaccgtctcctca

SEQ ID NO: 674 - protein, 127 aa, synthetic construct
  REGION 1..127: note = Synthetic
QVQLVQSGPEVKNPGASVKVSCKASGYTFTTYGISWVRQAPGQGLEWMGWISGYNGKTNDAQKFQDRVAMTTDTSTSTAYMELRSLRSDDTAIYYCSRDRLVVPPALYYYYYVMDVWGQGTTVTVSS

SEQ ID NO: 675 - DNA, 24 bp, synthetic construct
  misc_feature 1..24: note = Synthetic
  source 1..24: mol_type = other DNA
ggttacacctttaccacctatggt

SEQ ID NO: 676 - protein, 8 aa, synthetic construct
  REGION 1..8: note = Synthetic
GYTFTTYG

SEQ ID NO: 677 - DNA, 24 bp, synthetic construct
  misc_feature 1..24: note = Synthetic
  source 1..24: mol_type = other DNA
atcagcggttacaatggtaaaaca

SEQ ID NO: 678 - protein, 8 aa, synthetic construct
  REGION 1..8: note = Synthetic
ISGYNGKT

SEQ ID NO: 679 - DNA, 60 bp, synthetic construct
  misc_feature 1..60: note = Synthetic
  source 1..60: mol_type = other DNA
tcgagagatcgtttagtagtaccacctgccctttattattactactacgttatggacgtc

SEQ ID NO: 680 - protein, 20 aa, synthetic construct
  REGION 1..20: note = Synthetic
SRDRLVVPPALYYYYYVMDV

SEQ ID NO: 681 - DNA, 336 bp, synthetic construct; identical to SEQ ID NO: 633
  misc_feature 1..336: note = Synthetic
  source 1..336: mol_type = other DNA

SEQ ID NO: 682 - protein, 112 aa, synthetic construct; identical to SEQ ID NO: 336
  REGION 1..112: note = Synthetic

SEQ ID NO: 683 - DNA, 33 bp, synthetic construct; identical to SEQ ID NO: 323
  misc_feature 1..33: note = Synthetic
  source 1..33: mol_type = other DNA

SEQ ID NO: 684 - protein, 11 aa, synthetic construct
  REGION 1..11: note = Synthetic
QSLVYSDGNTY

SEQ ID NO: 685-686 - skipped (2 entries)

SEQ ID NO: 687 - DNA, 27 bp, synthetic construct
  misc_feature 1..27: note = Synthetic
  source 1..27: mol_type = other DNA
atgcaaggtacacactggccgtacact

SEQ ID NO: 688 - protein, 9 aa, synthetic construct
  REGION 1..9: note = Synthetic
MQGTHWPYT

SEQ ID NO: 689 - DNA, 381 bp, synthetic construct; identical to SEQ ID NO: 673
  misc_feature 1..381: note = Synthetic
  source 1..381: mol_type = other DNA

SEQ ID NO: 690 - protein, 127 aa, synthetic construct; identical to SEQ ID NO: 674
  REGION 1..127: note = Synthetic

SEQ ID NO: 691 - DNA, 336 bp, synthetic construct; identical to SEQ ID NO: 633
  misc_feature 1..336: note = Synthetic
  source 1..336: mol_type = other DNA

SEQ ID NO: 692 - protein, 112 aa, synthetic construct; identical to SEQ ID NO: 336
  REGION 1..112: note = Synthetic

SEQ ID NO: 693 - DNA, 381 bp, synthetic construct
  misc_feature 1..381: note = Synthetic
  source 1..381: mol_type = other DNA
caggttcagctggtgcagtctggagctgaggtgaagaagcctggggcctcagtgaaggtctcctgcaaggcttctggttacacctttaccacctatggtatcagctgggtgcgacaggcccctggacaagggcttgagtggatgggatggatcagcggttacaatggtaaaacaaactatgcacagaagctccagggcagagtcaccatgaccacagacacatccacgagcacagcctacatggagctgaggagcctgagatctgacgacacggccgtgtattactgttcgagagatcgtttagtagtaccacctgccctttattattactactacgttatggacgtctgggggcaagggaccacggtcaccgtctcctca

SEQ ID NO: 694 - protein, 127 aa, synthetic construct
  REGION 1..127: note = Synthetic
QVQLVQSGAEVKKPGASVKVSCKASGYTFTTYGISWVRQAPGQGLEWMGWISGYNGKTNYAQKLQGRVTMTTDTSTSTAYMELRSLRSDDTAVYYCSRDRLVVPPALYYYYYVMDVWGQGTTVTVSS

SEQ ID NO: 695 - DNA, 336 bp, synthetic construct; identical to SEQ ID NO: 335
  misc_feature 1..336: note = Synthetic
  source 1..336: mol_type = other DNA

SEQ ID NO: 696 - protein, 112 aa, synthetic construct; identical to SEQ ID NO: 336
  REGION 1..112: note = Synthetic

SEQ ID NO: 697 - DNA, 384 bp, synthetic construct; identical to SEQ ID NO: 361
  misc_feature 1..384: note = Synthetic
  source 1..384: mol_type = other DNA

SEQ ID NO: 698 - protein, 128 aa, synthetic construct; identical to SEQ ID NO: 362
  REGION 1..128: note = Synthetic

SEQ ID NO: 699 - DNA, 24 bp, synthetic construct
  misc_feature 1..24: note = Synthetic
  source 1..24: mol_type = other DNA
ggattcaccttcagtgaccactac

SEQ ID NO: 700 - protein, 8 aa, synthetic construct
  REGION 1..8: note = Synthetic
GFTFSDHY

SEQ ID NO: 701 - DNA, 24 bp, synthetic construct
  misc_feature 1..24: note = Synthetic
  source 1..24: mol_type = other DNA
attagtaatgatggtggtaccaaa

SEQ ID NO: 702 - protein, 8 aa, synthetic construct
  REGION 1..8: note = Synthetic
ISNDGGTK

SEQ ID NO: 703 - DNA, 63 bp, synthetic construct; identical to SEQ ID NO: 367
  misc_feature 1..63: note = Synthetic
  source 1..63: mol_type = other DNA

SEQ ID NO: 704 - protein, 21 aa, synthetic construct
  REGION 1..21: note = Synthetic
ARDQGYIGYDSYYYYSYGMDV

SEQ ID NO: 705 - DNA, 321 bp, synthetic construct
  misc_feature 1..321: note = Synthetic
  source 1..321: mol_type = other DNA
aaaattgtgttgacgcagtctccaggcaccctgcctttgtttccaggggaaagagccaccctctcctgtagggccagtcagagtgttaacaacaaattcttagcctggtaccagcagaaatctggccaggctcccaggctcctcatctatggtgcatccagcagggccactggcatcccagacaggttcagtggcagtgggtctgggaccgacttcactctcaccatcagcggactggagcctgaagattttgaagtgtattattgtcaagtatatggtaactcactcactttcggcggagggaccaaggtggagatcaag

SEQ ID NO: 706 - protein, 107 aa, synthetic construct
  REGION 1..107: note = Synthetic
KIVLTQSPGTLPLFPGERATLSCRASQSVNNKFLAWYQQKSGQAPRLLIYGASSRATGIPDRFSGSGSGTDFTLTISGLEPEDFEVYYCQVYGNSLTFGGGTKVEIK

SEQ ID NO: 707 - DNA, 21 bp, synthetic construct
  misc_feature 1..21: note = Synthetic
  source 1..21: mol_type = other DNA
cagagtgttaacaacaaattc

SEQ ID NO: 708 - protein, 7 aa, synthetic construct
  REGION 1..7: note = Synthetic
QSVNNKF

SEQ ID NO: 709-710 - skipped (2 entries)

SEQ ID NO: 711 - DNA, 24 bp, synthetic construct
  misc_feature 1..24: note = Synthetic
  source 1..24: mol_type = other DNA
caagtatatggtaactcactcact

SEQ ID NO: 712 - protein, 8 aa, synthetic construct
  REGION 1..8: note = Synthetic
QVYGNSLT

SEQ ID NO: 713 - DNA, 384 bp, synthetic construct; identical to SEQ ID NO: 377
  misc_feature 1..384: note = Synthetic
  source 1..384: mol_type = other DNA

SEQ ID NO: 714 - protein, 128 aa, synthetic construct; identical to SEQ ID NO: 378
  REGION 1..128: note = Synthetic

SEQ ID NO: 715 - DNA, 321 bp, synthetic construct
  misc_feature 1..321: note = Synthetic
  source 1..321: mol_type = other DNA
gaaattgtgttgacgcagtctccaggcaccctgcctttgtttccaggggaaagagccaccctctcctgtagggccagtcagagtgttaacaacaaattcttagcctggtaccagcagaaatctggccaggctcccaggctcctcatctatggtgcatccagcagggccactggcatcccagacaggttcagtggcagtgggtctgggaccgacttcactctcaccatcagcggactggagcctgaagattttgaagtgtattattgtcaagtatatggtaactcactcactttcggcggagggaccaaggtggagatcaaa

SEQ ID NO: 716 - protein, 107 aa, synthetic construct
  REGION 1..107: note = Synthetic
EIVLTQSPGTLPLFPGERATLSCRASQSVNNKFLAWYQQKSGQAPRLLIYGASSRATGIPDRFSGSGSGTDFTLTISGLEPEDFEVYYCQVYGNSLTFGGGTKVEIK

SEQ ID NO: 717 - DNA, 384 bp, synthetic construct; identical to SEQ ID NO: 381
  misc_feature 1..384: note = Synthetic
  source 1..384: mol_type = other DNA

SEQ ID NO: 718 - protein, 128 aa, synthetic construct; identical to SEQ ID NO: 382
  REGION 1..128: note = Synthetic

SEQ ID NO: 719 - DNA, 321 bp, synthetic construct; identical to SEQ ID NO: 383
  misc_feature 1..321: note = Synthetic
  source 1..321: mol_type = other DNA

SEQ ID NO: 720 - protein, 107 aa, synthetic construct; identical to SEQ ID NO: 384
  REGION 1..107: note = Synthetic

SEQ ID NO: 721 - DNA, 378 bp, synthetic construct; identical to SEQ ID NO: 505
  misc_feature 1..378: note = Synthetic
  source 1..378: mol_type = other DNA

SEQ ID NO: 722 - protein, 126 aa, synthetic construct; identical to SEQ ID NO: 506
  REGION 1..126: note = Synthetic

SEQ ID NO: 723 - DNA, 24 bp, synthetic construct
  misc_feature 1..24: note = Synthetic
  source 1..24: mol_type = other DNA
ggttacacctttaccaactacgct

SEQ ID NO: 724 - protein, 8 aa, synthetic construct
  REGION 1..8: note = Synthetic
GYTFTNYA

SEQ ID NO: 725 - DNA, 24 bp, synthetic construct
  misc_feature 1..24: note = Synthetic
  source 1..24: mol_type = other DNA
gtcagcgcttacaatggtcacaca

SEQ ID NO: 726 - protein, 8 aa, synthetic construct
  REGION 1..8: note = Synthetic
VSAYNGHT

SEQ ID NO: 727 - DNA, 57 bp, synthetic construct; identical to SEQ ID NO: 511
  misc_feature 1..57: note = Synthetic
  source 1..57: mol_type = other DNA

SEQ ID NO: 728 - protein, 19 aa, synthetic construct
  REGION 1..19: note = Synthetic
ARGGVVVPVAPHFYNGMDV

SEQ ID NO: 729 - DNA, 336 bp, synthetic construct
  misc_feature 1..336: note = Synthetic
  source 1..336: mol_type = other DNA
gatattgtgatgactcagtttccactctccctgcccgtcacccctggagagccggcctccatctcctgcaggtctagtcagagcctcctgcatattaatgaatacaactatttggattggtacctaaagaagccagggcagtctccacagctcctgatctatttgggttttaatcgggcctccggggtccctgacaggttcagtggcagtggatcaggcacagattttacactgaaaatcagcagagtggaggctgaggatgttggggtctattactgcatgcaagctcttcaaactccgtggacgttcggccaagggaccaaggtggaaatcaaa

SEQ ID NO: 730 - protein, 112 aa, synthetic construct
  REGION 1..112: note = Synthetic
DIVMTQFPLSLPVTPGEPASISCRSSQSLLHINEYNYLDWYLKKPGQSPQLLIYLGFNRASGVPDRFSGSGSGTDFTLKISRVEAEDVGVYYCMQALQTPWTFGQGTKVEIK

SEQ ID NO: 731 - DNA, 33 bp, synthetic construct; identical to SEQ ID NO: 515
  misc_feature 1..33: note = Synthetic
  source 1..33: mol_type = other DNA

SEQ ID NO: 732 - protein, 11 aa, synthetic construct
  REGION 1..11: note = Synthetic
QSLLHINEYNY

SEQ ID NO: 733-734 - skipped (2 entries)

SEQ ID NO: 735 - DNA, 27 bp, synthetic construct
  misc_feature 1..27: note = Synthetic
  source 1..27: mol_type = other DNA
atgcaagctcttcaaactccgtggacg

SEQ ID NO: 736 - protein, 9 aa, synthetic construct
  REGION 1..9: note = Synthetic
MQALQTPWT

SEQ ID NO: 737 - DNA, 378 bp, synthetic construct; identical to SEQ ID NO: 521
  misc_feature 1..378: note = Synthetic
  source 1..378: mol_type = other DNA

SEQ ID NO: 738 - protein, 126 aa, synthetic construct; identical to SEQ ID NO: 522
  REGION 1..126: note = Synthetic

SEQ ID NO: 739 - DNA, 336 bp, synthetic construct
  misc_feature 1..336: note = Synthetic
  source 1..336: mol_type = other DNA
gatattgtgatgactcagtctccactctccctgcccgtcacccctggagagccggcctccatctcctgcaggtctagtcagagcctcctgcatattaatgaatacaactatttggattggtacctaaagaagccagggcagtctccacagctcctgatctatttgggttttaatcgggcctccggggtccctgacaggttcagtggcagtggatcaggcacagattttacactgaaaatcagcagagtggaggctgaggatgttggggtctattactgcatgcaagctcttcaaactccgtggacgttcggccaagggaccaaggtggaaatcaaa

SEQ ID NO: 740 - protein, 112 aa, synthetic construct
  REGION 1..112: note = Synthetic
DIVMTQSPLSLPVTPGEPASISCRSSQSLLHINEYNYLDWYLKKPGQSPQLLIYLGFNRASGVPDRFSGSGSGTDFTLKISRVEAEDVGVYYCMQALQTPWTFGQGTKVEIK

SEQ ID NO: 741 - DNA, 378 bp, synthetic construct; identical to SEQ ID NO: 525
  misc_feature 1..378: note = Synthetic
  source 1..378: mol_type = other DNA

SEQ ID NO: 742 - protein, 126 aa, synthetic construct; identical to SEQ ID NO: 526
  REGION 1..126: note = Synthetic

SEQ ID NO: 743 - DNA, 336 bp, synthetic construct; identical to SEQ ID NO: 527
  misc_feature 1..336: note = Synthetic
  source 1..336: mol_type = other DNA

SEQ ID NO: 744 - protein, 112 aa, synthetic construct; identical to SEQ ID NO: 528
  REGION 1..112: note = Synthetic

SEQ ID NO: 745-750 - skipped (6 entries)

SEQ ID NO: 751 - protein, 330 aa, synthetic construct [sequence omitted]

SEQ ID NO: 752 - protein, 327 aa, synthetic construct
  REGION 1..327: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 753 - protein, 327 aa, synthetic construct
  REGION 1..327: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 754 - DNA, 2076 bp, Homo sapiens
  source 1..2076: mol_type = genomic DNA
atgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtgtggtcagcacactcggggcctacacggatggccacagccatcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaagaggccgtgacagccgttgccatctgctgccggagccggcacctggcgcaggcctcccaggagctccag

SEQ ID NO: 755 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEEAVTAVAICCRSRHLAQASQELQ

SEQ ID NO: 756 - protein, 692 aa, Macaca mulatta
MGTVSSRRSWWPLPLPLLLLLLLGPAGARAQEDEDGDYEELVLALRSEEDGLADAPEHGATATFHRCAKDPWRLPGTYVVVLKEETHRSQSERTARRLQAQAARRGYLTKILHVFHHLLPGFLVKMSGDLLELALKLPHVDYIEEDSSVFAQSIPWNLERITPARYRADEYQPPKGGSLVEVYLLDTSIQSDHREIEGRVMVTDFESVPEEDGTRFHRQASKCDSHGTHLAGVVSGRDAGVAKGAGLRSLRVLNCQGKGTVSGTLIGLEFIRKSQLVQPVGPLVVLLPLAGGYSRVFNAACQRLARAGVVLVTAAGNFRDDACLYSPASAPEVITVGATNAQDQPVTLGTLGTNFGRCVDLFAPGEDIIGASSDCSTCFVSRSGTSQAAAHVAGIAAMMLSAEPELTLAELRQRLIHFSAKDVINEAWFPEDQRVLTPNLVAALPPSTHRAGWQLFCRTVWSAHSGPTRMATAVARCAQDEELLSCSSFSRSGKRRGERIEAQGGKRVCRAHNAFGGEGVYAIARCCLLPQVNCSVHTAPPAGASMGTRVHCHQQGHVLTGCSSHWEVEDLGTHKPPVLRPRGQPNQCVGHREASIHASCCHAPGLECKVKEHGIPAPQEQVIVACEDGWTLTGCSPLPGTSHVLGAYAVDNTCVVRSRDVSTTGSTSKEAVAAVAICCRSRHLVQASQELQ

SEQ ID NO: 757 - protein, 694 aa, Mus musculus
MGTHCSAWLRWPLLPLLPPLLLLLLLLCPTGAGAQDEDGDYEELMLALPSQEDGLADEAAHVATATFRRCSKEAWRLPGTYIVVLMEETQRLQIEQTAHRLQTRAARRGYVIKVLHIFYDLFPGFLVKMSSDLLGLALKLPHVEYIEEDSFVFAQSIPWNLERIIPAWHQTEEDRSPDGSSQVEVYLLDTSIQGAHREIEGRVTITDFNSVPEEDGTRFHRQASKCDSHGTHLAGVVSGRDAGVAKGTSLHSLRVLNCQGKGTVSGTLIGLEFIRKSQLIQPSGPLVVLLPLAGGYSRILNAACRHLARTGVVLVAAAGNFRDDACLYSPASAPEVITVGATNAQDQPVTLGTLGTNFGRCVDLFAPGKDIIGASSDCSTCFMSQSGTSQAAAHVAGIVARMLSREPTLTLAELRQRLIHFSTKDVINMAWFPEDQQVLTPNLVATLPPSTHETGGQLLCRTVWSAHSGPTRTATATARCAPEEELLSCSSFSRSGRRRGDWIEAIGGQQVCKALNAFGGEGVYAVARCCLVPRANCSIHNTPAARAGLETHVHCHQKDHVLTGCSFHWEVEDLSVRRQPALRSRRQPGQCVGHQAASVYASCCHAPGLECKIKEHGISGPSEQVTVACEAGWTLTGCNVLPGASLTLGAYSVDNLCVARVHDTARADRTSGEATVAAAICCRSRPSAKASWVQ

SEQ ID NO: 758 - protein, 653 aa, Homo sapiens
EFRCHDGKCISRQFVCDSDRDCLDGSDEASCPVLTCGPASFQCNSSTCIPQLWACDNDPDCEDGSDEWPQRCRGLYVFQGDSSPCSAFEFHCLSGECIHSSWRCDGGPDCKDKSDEENCAVATCRPDEFQCSDGNCIHGSRQCDREYDCKDMSDEVGCVNVTLCEGPNKFKCHSGECITLDKVCNMARDCRDWSDEPIKECGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLTEAEAAVATQETSTVRLKVSSTAVRTQHTTTRPVPDTSRLPGATPGLTTVEIV

SEQ ID NO: 759 - protein, 753 aa, Homo sapiens
MERRAWSLQCTAFVLFCAWCALNSAKAKRQFVNEWAAEIPGGPEAASAIAEELGYDLLGQIGSLENHYLFKHKNHPRRSRRSAFHITKRLSDDDRVIWAEQQYEKERSKRSALRDSALNLFNDPMWNQQWYLQDTRMTAALPKLDLHVIPVWQKGITGKGVVITVLDDGLEWNHTDIYANYDPEASYDFNDNDHDPFPRYDPTNENKHGTRCAGEIAMQANNHKCGVGVAYNSKVGGIRMLDGIVTDAIEASSIGFNPGHVDIYSASWGPNDDGKTVEGPGRLAQKAFEYGVKQGRQGKGSIFVWASGNGGRQGDNCDCDGYTDSIYTISISSASQQGLSPWYAEKCSSTLATSYSSGDYTDQRITSADLHNDCTETHTGTSASAPLAAGIFALALEANPNLTWRDMQHLVVWTSEYDPLANNPGWKKNGAGLMVNSRFGFGLLNAKALVDLADPRTWRSVPEKKECVVKDNDFEPRALKANGEVIIEIPTRACEGQENAIKSLEHVQFEATIEYSRRGDLHVTLTSAAGTSTVLLAERERDTSPNGFKNWDFMSVHTWGENPIGTWTLRITDMSGRIQNEGRIVNWKLILHGTSSQPEHMKQPRVYTSYNTVQNDRRGVEKMVDPGEEQPTQENPKENTLVSKSPSSSSVGGRRDELEEGAPSQAMLRLLQSAFSKNSPPKQSPKKSPSAKLNIPYENFYEALEKLNKPSQLKDSEDSLYNDYVDVFYNTKPYKHRDDRLLQALVDILNEEN

SEQ ID NO: 760 - protein, 785 aa, Homo sapiens
MPKGRQKVPHLDAPLGLPTCLWLELAGLFLLVPWVMGLAGTGGPDGQGTGGPSWAVHLESLEGDGEEETLEQQADALAQAAGLVNAGRIGELQGHYLFVQPAGHRPALEVEAIRQQVEAVLAGHEAVRWHSEQRLLRRAKRSVHFNDPKYPQQWHLNNRRSPGRDINVTGVWERNVTGRGVTVVVVDDGVEHTIQDIAPNYSPEGSYDLNSNDPDPMPHPDVENGNHHGTRCAGEIAAVPNNSFCAVGVAYGSRIAGIRVLDGPLTDSMEAVAFNKHYQINDIYSCSWGPDDDGKTVDGPHQLGKAALQHGVIAGRQGFGSIFVVASGNGGQHNDNCNYDGYANSIYTVTIGAVDEEGRMPFYAEECASMLAVTFSGGDKMLRSIVTTDWDLQKGTGCTEGHTGTSAAAPLAAGMIALMLQVRPCLTWRDVQHIIVFTATRYEDRRAEWVTNEAGFSHSHQHGFGLLNAWRLVNAAKIWTSVPYLASYVSPVLKENKAIPQSPRSLEVLWNVSRMDLEMSGLKTLEHVAVTVSITHPRRGSLELKLFCPSGMMSLIGAPRSMDSDPNGFNDWTFSTVRCWGERARGTYRLVIRDVGDESFQVGILRQWQLTLYGSVWSAVDIRDRQRLLESAMSGKYLHDDFALPCPPGLKIPEEDGYTITPNTLKTLVLVGCFTVFWTVYYMLEVYLSQRNVASNQVCRSGPCHWPHRSRKAKEEGTELESVPLCSSKDPDEVETESRGPPTTSDLLAPDLLEQGDWSLSQNKSALDCPHQHLDVPHGKEEQIC

SEQ ID NO: 761 - protein, 692 aa, Macaca fascicularis
MGTVSSRRSWWPLPLPLLLLLLLGPAGARAQEDEDGDYEELVLALRSEEDGLADAPEHGATATFHRCAKDPWRLPGTYVVVLKEETHRSQSERTARRLQAQAARRGYLTKILHVFHHLLPGFLVKMSGDLLELALKLPHVDYIEEDSSVFAQSIPWNLERITPARYRADEYQPPKGGSLVEVYLLDTSIQSDHREIEGRVMVTDFESVPEEDGTRFHRQASKCDSHGTHLAGVVSGRDAGVAKGAGLRSLRVLNCQGKGTVSGTLIGLEFIRKSQLVQPVGPLVVLLPLAGGYSRVFNAACQRLARAGVVLVTAAGNFRDDACLYSPASAPEVITVGATNAQDQPVTLGTLGTNFGRCVDLFAPGEDIIGASSDCSTCFVSRSGTSQAAAHVAGIAAMMLSAEPELTLAELRQRLIHFSAKDVINEAWFPEDQRVLTPNLVAALPPSTHRAGWQLFCRTVWSAHSGPTRMATAVARCAQDEELLSCSSFSRSGKRRGERIEAQGGKRVCRAHNAFGGEGVYAIARCCLLPQVNCSVHTAPPAGASMGTRVHCHQQGHVLTGCSSHWEVEDLGTHKPPVLRPRGQPNQCVGHREASIHASCCHAPGLECKVKEHGIPAPQEQVIVACEDGWTLTGCSALPGTSHVLGAYAVDNTCVVRSRDVSTTGSTSEEAVAAVAICCRSRHLVQASQELQ

SEQ ID NO: 762 - protein, 698 aa, Mesocricetus auratus
MGTSCSARPRWLLSPLLLLLLLLRYMGASAQDEDAEYEELMLTLQSQDDGLADETDEAPQGATAAFHRCPEEAWRVPGTYIVMLAEEAQWVHIEQTMHRLQTQAARRGYVIKIQHIFYDFLPAFVVKMSSDLLDLALKLPHVKYIEEDSLVFAQSIPWNLDRIIPAGRQAQEYSSSRKVPSGSGQVEVYLLDTSIQSDHREIEGRVTVTDFNSVPEEDGTRFHRQASKCDSHGTHLAGVVSGRDAGVAKGTILHGLRVLNCQGKGIVSGILTGLEFIWKSQLMQPSGPQVVLLPLAGRYSRVLNTACQHLARTGVVLVAAAGNFRDDACLYSPASAPEVITVGATDVQDQPVTLGTLGTNFGRCVDLFAPGKDIIGASSDCSACFMSQSGTSQAAAHVAGIVAMMLTLEPELTLTELRQRLIHFSTKDAINMAWFPEDQRVLTPNLVATLPPSTHGTGGQLLCRTVWSAHSGPTRAATATARCAPGEELLSCSSFSRSGRRRGDRIEAAGTQQVCKALNAFGGEGVYAVARCCLLPRANCSIHTTPAARTSLETHAHCHQKDHVLTGCSLHWEVEGIGVQPLAVLRSRHQPGQCTGHREASVHASCCHAPGLECKIKEHGISGPAEQVTVACEAGWTLTGCNVLPGAFITLGAYAVDNTCVARSRVTDTAGRTGEEATVAAAICCRNRPSAKASWVHQ

SEQ ID NO: 763 - protein, 691 aa, Rattus norvegicus
MGIRCSTWLRWPLSPQLLLLLLLCPTGSRAQDEDGDYEELMLALPSQEDSLVDEASHVATATFRRCSKEAWRLPGTYVVVLMEETQRLQVEQTAHRLQTWAARRGYVIKVLHVFYDLFPGFLVKMSSDLLGLALKLPHVEYIEEDSLVFAQSIPWNLERIIPAWQQTEEDSSPDGSSQVEVYLLDTSIQSGHREIEGRVTITDFNSVPEEDGTRFHRQASKCDSHGTHLAGVVSGRDAGVAKGTSLHSLRVLNCQGKGTVSGTLIGLEFIRKSQLIQPSGPLVVLLPLAGGYSRILNTACQRLARTGVVLVAAAGNFRDDACLYSPASAPEVITVGATNAQDQPVTLGTLGTNFGRCVDLFAPGKDIIGASSDCSTCYMSQSGTSQAAAHVAGIVAMMLNRDPALTLAELRQRLILFSTKDVINMAWFPEDQRVLTPNRVATLPPSTQETGGQLLCRTVWSAHSGPTRTATATARCAPEEELLSCSSFSRSGRRRGDRIEAIGGQQVCKALNAFGGEGVYAVARCCLLPRVNCSIHNTPAARAGPQTPVHCHQKDHVLTGCSFHWEVENLRAQQQPLLRSRHQPGQCVGHQEASVHASCCHAPGLECKIKEHGIAGPAEQVTVACEAGWTLTGCNVLPGASLPLGAYSVDNVCVARIRDAGRADRTSEEATVAAAICCRSRPSAKASWVHQ

(19) United States
(12) Patent Application Publication
(10) Pub. No.: US 2025/0154286 A1
(43) Pub. Date: May 15, 2025

(54) ANTI-PCSK9 ANTIBODIES

(71) Applicant: REGENERON PHARMACEUTICALS, INC., Tarrytown, NY (US)

(72) Inventors: Mark W. SLEEMAN, Victoria (AU); Joel H. MARTIN, Putnam Valley, NY (US); Tammy T. HUANG, Cross River, NY (US); Douglas MACDONALD, New York, NY (US)

(21) Appl. No.: 19/025,495

(22) Filed: Jan. 16, 2025

Related U.S. Application Data
(63) Continuation of application No. 18/296,265, filed on Apr. 5, 2023.
Application No. 18/296,265 is a continuation of application No. 17/160,634, filed on Jan. 28, 2021, now abandoned.
Application No. 17/160,634 is a continuation of application No. 15/996,773, filed on Jun. 4, 2018, now Pat. No. 10,941,210.
Application No. 15/996,773 is a continuation of application No. 15/377,364, filed on Dec. 13, 2016, now Pat. No. 10,023,654.
Application No. 15/377,364 is a continuation of application No. 14/737,488, filed on Jun. 12, 2015, now Pat. No. 9,550,837.
Application No. 14/737,488 is a continuation of application No. 13/690,585, filed on Nov. 30, 2012, now abandoned.
Application No. 13/690,585 is a continuation of application No. 12/949,846, filed on Nov. 19, 2010, now Pat. No. 8,501,184.
Application No. 12/949,846 is a division of application No. 12/637,942, filed on Dec. 15, 2009, now Pat. No. 8,062,640.
(60) Provisional application No. 61/261,776, filed on Nov. 17, 2009.
(60) Provisional application No. 61/249,135, filed on Oct. 6, 2009.
(60) Provisional application No. 61/218,136, filed on Jun. 18, 2009.
(60) Provisional application No. 61/168,753, filed on Apr. 13, 2009.
(60) Provisional application No. 61/210,566, filed on Mar. 18, 2009.
(60) Provisional application No. 61/122,482, filed on Dec. 15, 2008.

(51) Int. Cl. C07K 16/40; A61K 39/00; A61K 39/08; C07K 16/12; C12N 15/09; G01N 33/569

(52) U.S. Cl. CPC C07K 16/40; A61K 39/08; C07K 16/1282; C12N 15/09; C12Y 304/21061; G01N 33/56911; A61K 2039/505; A61K 2039/507; C07K 2317/21; C07K 2317/33; C07K 2317/34; C07K 2317/56; C07K 2317/565; C07K 2317/76; C07K 2317/92; G01N 2333/33; Y10S 424/809

(57) ABSTRACT

An human antibody or antigen-binding fragment of a human antibody that specifically binds and inhibits human proprotein convertase subtilisin/kexin type 9 (hPCSK9) characterized by the ability to reduce serum LDL cholesterol by 40-80% over a 24, 60 or 90 day period relative to predose levels, with little or no reduction in serum HDL cholesterol and/or with little or no measurable effect on liver function, as determined by ALT and AST measurements.

### CROSS-REFERENCE TO RELATED APPLICATIONS

This application is a continuation of U.S. patent application Ser. No. 18/296,265, filed Apr. 5, 2023, which is a continuation of U.S. patent application Ser. No. 17/160,634, filed Jan. 28, 2021, which is a continuation of U.S. patent application Ser. No. 15/996,773, filed Jun. 4, 2018, now U.S. Pat. No. 10,941,210, which is a continuation of U.S. patent application Ser. No. 15/377,364, filed Dec. 13, 2016, now U.S. Pat. No. 10,023,654, which is a continuation of U.S. patent application Ser. No. 14/737,488, filed Jun. 12, 2015, now. U.S. Pat. No. 9,550,837, which is a continuation of U.S. patent application Ser. No. 13/690,585, filed Nov. 30, 2012, which is a continuation of U.S. patent application Ser. No. 12/949,846, filed Nov. 19, 2010, now U.S. Pat. No. 8,501,184, which is a division of U.S. patent application Ser. No. 12/637,942, filed Dec. 15, 2009, now U.S. Pat. No. 8,062,640, which claims priority to U.S. Provisional Patent Application Ser. Nos. 61/261,776, filed Nov. 17, 2009,61/249,135, filed Oct. 6, 2009, 61/218,136, filed Jun. 18, 2009, 61/168,753, filed Apr. 13, 2009, 61/210,566, filed Mar. 18, 2009, and 61/122,482, filed Dec. 15, 2008, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Jan. 15, 2025, is named 761244_SA9-151DIVCON7_ST26.xml and is 969,645 bytes in size.

### FIELD OF THE INVENTION

The present invention is related to human antibodies and antigen-binding fragments of human antibodies that specifically bind human proprotein convertase subtilisin/kexin type 9 (PCSK9), and therapeutic methods of using those antibodies.

### STATEMENT OF RELATED ART

Proprotein convertase subtilisin/kexin type 9 (PCSK9) is a proprotein convertase belonging to the proteinase K subfamily of the secretory subtilase family. The encoded protein is synthesized as a soluble zymogen that undergoes autocatalytic intramolecular processing in the endoplasmic reticulum. Evidence suggest that PCSK9 increases plasma LDL cholesterol by promoting degradation of the LDL receptor, which mediates LDL endocytosis in the liver, the major route of LDL clearance from circulation. The structure of PCSK9 protein shows that it has a signal sequence, followed by a prodomain, a catalytic domain that contains a conserved triad of residues (D186, H226 and S386), and a C-terminal domain. It is synthesized as a soluble 74-kDa precursor that undergoes autocatalytic cleavage in the ER, generating a 14-kDa prodomain and 60-kDa catalytic fragment. The autocatalytic activity has been shown to be required for secretion. After cleavage, the prodomain remains tightly associated with the catalytic domain.

Antibodies to PCSK9 are described in, for example, WO 2008/057457, WO 2008/057458, WO 2008/057459, WO 2008/063382, WO 2008/125623, and US 2008/0008697.

### BRIEF SUMMARY OF THE INVENTION

In a first aspect, the invention provides fully human monoclonal antibodies (mAbs) and antigen-binding fragments thereof that specifically bind and neutralize human PCSK9 (hPCSK9) activity.

In one embodiment, the invention comprises an antibody or antigen-binding fragment of an antibody that specifically binds hPCSK9 and is characterized by at least one of:
(i) capable of reducing serum total cholesterol at least about 25-35% and sustaining the reduction over at least a 24 day period relative to a predose level, preferably the reduction in serum total cholesterol is at least about 30-40%;
(ii) capable of reducing serum LDL cholesterol at least about 65-80% and sustaining the reduction over at least a 24-day period relative to a predose level;
(iii) capable of reducing serum triglyceride at least about 25-40% relative to predose level;
(iv) does not reduce serum HDL cholesterol or reduces serum HDL cholesterol no more than 5% relative to predose level.

In one embodiment, the invention comprises an antibody or antigen-binding fragment of an antibody that specifically binds hPCSK9 and is characterized by at least one of:
(i) capable of reducing serum LDL cholesterol at least about 40-70% and sustaining the reduction over at least a 60 or 90 day period relative to a predose level;
(ii) capable of reducing serum triglyceride at least about 25-40% relative to predose level;
(iii) does not reduce serum HDL cholesterol or reduces serum HDL cholesterol no more than 5% relative to predose level.

In one embodiment, the antibody or antigen-binding fragment is characterized as binding an epitope comprising amino acid residue 238 of hPCSK9 (SEQ ID NO:755). In a more specific embodiment, the antibody or antigen-binding fragment binds an epitope comprising one or more of amino acid residue 238, 153, 159 and 343 of hPCSK9 (SEQ ID NO: 755).). In a more specific embodiment, the antibody or fragment thereof is characterized as binding an epitope that does not comprise an amino acid residue at position 192, 194, 197 and/or 237 of SEQ ID NO:755.

In one embodiment, the antibody or antigen-binding fragment is characterized as binding an epitope comprising amino acid residue 366 of hPCSK9 (SEQ ID NO:755). In a more specific embodiment, the antibody or antigen-binding fragment binds an epitope comprising one or more of amino acid residue at position 147, 366 and 380 of SEQ ID NO: 755. In a more specific embodiment, the antibody or antigen-binding fragment of an antibody is characterized as binding an epitope that does not comprise an amino acid residue at position 215 or 238 of SEQ ID NO:755.

In one embodiment, the antibody or antigen-binding fragment is characterized as exhibiting an enhanced binding affinity (K_{D}) for hPCSK9 at pH 5.5 relative to the K_{D} at pH 7.4, as measured by plasmon surface resonance. In a specific embodiment, the antibody or fragment thereof exhibits at least a 20-fold, at least a 40-fold or at least a 50-fold enhanced affinity for PCSK9 at an acidic pH relative to a neutral pH, as measured by surface plasmon resonance.

In one embodiment, the antibody or antigen-binding fragment is characterized as not exhibiting an enhanced binding affinity for PCSK9 at an acidic pH relative to a neutral pH, as measured by surface plasmon resonance. In a specific embodiment, the antibody or fragment thereof exhibits a decreased binding affinity at an acidic pH.

In another embodiment, the antibody or antigen-binding fragment binds human, human GOF mutation D374Y, cynomolgus monkey, rhesus monkey, mouse, rat and hamster PCSK9.

In one embodiment, the antibody or antigen-binding fragment binds human and monkey PCSK9, but does not bind mouse, rat or hamster PCSK9.

The mAbs can be full-length (e.g., an IgG1 or IgG4 antibody) or may comprise only an antigen-binding portion (e.g., a Fab, F(ab′)₂ or scFv fragment), and may be modified to affect functionality, e.g., to eliminate residual effector functions (Reddy et al. (2000) J. Immunol. 164:1925-1933).

In one embodiment, the invention comprises an antibody or antigen-binding fragment of an antibody comprising a heavy chain variable region (HCVR) selected from the group consisting of SEQ ID NO:2, 18, 22, 26, 42, 46, 50, 66, 70, 74, 90, 94, 98, 114, 118, 122, 138, 142, 146, 162, 166, 170, 186, 190, 194, 210, 214, 218, 234, 238, 242, 258, 262, 266, 282, 286, 290, 306, 310, 314, 330, 334, 338, 354, 358, 362, 378, 382, 386, 402, 406, 410, 426, 430, 434, 450, 454, 458, 474, 478, 482, 498, 502, 506, 522, 526, 530, 546, 550, 554, 570, 574, 578, 594, 598, 602, 618, 622, 626, 642, 646, 650, 666, 670, 674, 690, 694, 698, 714, 718, 722, 738 and 742, or a substantially similar sequence thereof having at least 90%, at least 95%, at least 98% or at least 99% sequence identity. In one embodiment, the HCVR comprises an amino acid sequence selected from the group consisting of SEQ ID NO:50, 66, 70, 74, 90, 94, 122, 138, 142, 218, 234, 238, 242, 258, 262, 314, 330 and 334. In a more specific embodiment, the HCVR comprises SEQ ID NO: 90 or 218.

In one embodiment, the antibody or fragment thereof further comprises a light chain variable region (LCVR) selected from the group consisting of SEQ ID NO:10, 20, 24, 34, 44, 48, 58, 68, 72, 82, 92, 96, 106, 116, 120, 130, 140, 144, 154, 164, 168, 178, 188, 192, 202, 212, 216, 226, 236, 240, 250, 260, 264, 274, 284, 288, 298, 308, 312, 322, 332, 336, 346, 356, 360, 370, 380, 384, 394, 404, 408, 418, 428, 432, 442, 452, 456, 466, 476, 480, 490, 500, 504, 514, 524, 528, 538, 548, 552, 562, 572, 576, 586, 596, 600, 610, 620, 624, 634, 644, 648, 658, 668, 672, 682, 692, 696, 706, 716, 720, 730, 740 and 744, or a substantially similar sequence thereof having at least 90%, at least 95%, at least 98% or at least 99% sequence identity. In one embodiment, the LCVR comprises an amino acid sequence selected from the group consisting of SEQ ID NO: 58, 68, 72, 82, 92, 96, 130, 140, 144, 226, 236, 240, 250, 260, 264, 322, 332 and 336. In a more specific embodiment, the LCVR comprises SEQ ID NO:92 or 226.

In specific embodiments, the antibody or fragment thereof comprises a HCVR and LCVR (HCVR/LCVR) sequence pair selected from the group consisting of SEQ ID NO: 2/10, 18/20, 22/24, 26/34, 42/44, 46/48, 50/58, 66/68, 70/72, 74/82, 90/92, 94/96, 98/106, 114/116, 118/120, 122/130, 138/140, 142/144, 146/154, 162/164, 166/168, 170/178, 186/188, 190/192, 194/202, 210/212, 214/216, 218/226, 234/236, 238/240, 242/250, 258/260, 262/264, 266/274, 282/284, 286/288, 290/298, 306/308, 310/312, 314/322, 330/332, 334/336, 338/346, 354/356, 358/360, 362/370, 378/380, 382/384, 386/394, 402/404, 406/408, 410/418, 426/428, 430/432, 434/442, 450/452, 454/456, 458/466, 474/476, 478/480, 482/490, 498/500, 502/504, 506/514, 522/524, 526/528, 530/538, 546/548, 550/552, 554/562, 570/572, 574/576, 578/586, 594/596, 598/600, 602/610, 618/620, 622/624, 626/634, 642/644, 646/648, 650/658, 666/668, 670/672, 674/682, 690/692, 694/696, 698/706, 714/716, 718/720, 722/730, 738/740 and 742/744. In one embodiment, the HCVR and LCVR sequence pair comprises one of SEQ ID NO: 50/58, 66/68, 70/72, 74/82, 90/92, 94/96, 122/130, 138/140, 142/144, 218/226, 234/236, 238/240, 242/250, 258/260, 262/264, 314/322, 330/332 and 334/336. In a more specific embodiment, the HCVR/LCVR pair comprises SEQ ID NO:90/92 or 218/226.

In a second aspect, the invention features an antibody or antigen-binding fragment of an antibody comprising a heavy chain CDR3 (HCDR3) domain selected from the group consisting of SEQ ID NO:8, 32, 56, 80, 104, 128, 152, 176, 200, 224, 248, 272, 296, 320, 344, 368, 392, 416, 440, 464, 488, 512, 536, 560, 584, 608, 632, 656, 680, 704 and 728, or a substantially similar sequence thereof having at least 90%, at least 95%, at least 98% or at least 99% sequence identity; and a light chain CDR3 (LCDR3) domain selected from the group consisting of SEQ ID NO:16, 40, 64, 88, 112, 136, 160, 184, 208, 232, 256, 280, 304, 328, 352, 376, 400, 424, 448, 472, 496, 520, 544, 568, 592, 616, 640, 664, 688, 712 and 736, or substantially similar sequences thereof having at least 90%, at least 95%, at least 98% or at least 99% sequence identity. In one embodiment, the HCDR3/LCDR3 sequence pair is selected from the group consisting of SEQ ID NO:56/64, 80/88, 128/136, 224/232, 248/256 and 320/328. In a more specific embodiment, the HCDR3/LCDR3 sequence pair comprises SEQ ID NO:80/88 or 224/232.

In a further embodiment, the invention comprising an antibody or fragment thereof further comprising a heavy chain CDR1 (HCDR1) domain selected from the group consisting of SEQ ID NO:4, 28, 52, 76, 100, 124, 148, 172, 196, 220, 244, 268, 292, 316, 340, 364, 388, 412, 436, 460, 484, 508, 532, 556, 580, 604, 628, 652, 676, 700 and 724, or a substantially similar sequence thereof having at least 90%, at least 95%, at least 98% or at least 99% sequence identity; a heavy chain CDR2 (HCDR2) domain selected from the group consisting of SEQ ID NO:6, 30, 54, 78, 102, 126, 150, 174, 198, 222, 246, 270, 294, 318, 342, 366, 390, 414, 438, 462, 486, 510, 534, 558, 582, 606, 630, 654, 678, 702 and 726, or a substantially similar sequence thereof having at least 90%, at least 95%, at least 98% or at least 99% sequence identity; a light chain CDR1 (LCDR1) domain selected from the group consisting of SEQ ID NO:12, 36, 60, 84, 108, 132, 156, 180, 204, 228, 252, 276, 300, 324, 348, 372, 396, 420, 444, 468, 492, 516, 540, 564, 588, 612, 636, 660, 684, 708 and 732, or a substantially similar sequence thereof having at least 90%, at least 95%, at least 98% or at least 99% sequence identity; and a light chain CDR2 (LCDR2) domain selected from the group consisting of SEQ ID NO:14, 38, 62, 86, 110, 134, 158, 182, 206, 254, 278, 302, 326, 350, 374, 398, 422, 446, 470, 494, 518, 542, 566, 590, 614, 638, 662, 686, 710, 734, and amino acid sequence LGS or a substantially similar sequence thereof having at least 90%, at least 95%, at least 98% or at least 99% sequence identity. In one embodiment, the heavy and light chain CDR sequences comprise a sequence selected from the group consisting of SEQ ID NO:52, 54, 56, 60, 62, 64; 76, 78, 80, 84, 86, 88; 124, 126, 128, 132, 134, 136; 220, 222, 224, 228, 232; 244, 246, 248, 252, 254, 256; and 316, 318, 320, 324, 326, 328 and amino acid sequence LGS. In more specific embodiments, the CDR sequences comprise SEQ ID NO: 76, 78, 80, 84, 86, 88; or 220, 222, 224, 228, 232 or amino acid sequence LGS.

In a related embodiment, the invention comprises an antibody or antigen-binding fragment of an antibody which specifically binds hPCSK9, wherein the antibody or fragment comprises heavy and light chain CDR domains contained within heavy and light chain sequence pairs selected from the group consisting of SEQ ID NO: 2/10, 18/20, 22/24, 26/34, 42/44, 46/48, 50/58, 66/68, 70/72, 74/82, 90/92, 94/96, 98/106, 114/116, 118/120, 122/130, 138/140, 142/144, 146/154, 162/164, 166/168, 170/178, 186/188, 190/192, 194/202, 210/212, 214/216, 218/226, 234/236, 238/240, 242/250, 258/260, 262/264, 266/274, 282/284, 286/288, 290/298, 306/308, 310/312, 314/322, 330/332, 334/336, 338/346, 354/356, 358/360, 362/370, 378/380, 382/384, 386/394, 402/404, 406/408, 410/418, 426/428, 430/432, 434/442, 450/452, 454/456, 458/466, 474/476, 478/480, 482/490, 498/500, 502/504, 506/514, 522/524, 526/528, 530/538, 546/548, 550/552, 554/562, 570/572, 574/576, 578/586, 594/596, 598/600, 602/610, 618/620, 622/624, 626/634, 642/644, 646/648, 650/658, 666/668, 670/672, 674/682, 690/692, 694/696, 698/706, 714/716, 718/720, 722/730, 738/740 and 742/744. In one embodiment, the CDR sequences are contained within HCVR and LCVR selected from the amino acid sequence pairs of SEQ ID NO: 50/58, 66/68, 70/72, 74/82, 90/92, 94/96, 122/130, 138/140, 142/144, 218/226, 234/236, 238/240, 242/250, 258/260, 262/264, 314/322, 330/332 and 334/336. In more specific embodiments, the CDR sequences are comprised within HCVR/LCVR sequences selected from SEQ ID NO: 90/92 or 218/226.

In one embodiment, the invention provides fully human monoclonal antibody or antigen-binding fragment thereof that specifically bind hPCSK9 and neutralize PCSK9 activity, wherein the antibody or fragment thereof exhibits one or more of the following characteristics: (i) capable of reducing serum total cholesterol at least about 25-35% and sustaining the reduction over at least a 24 day period relative to a predose level, preferably the reduction in serum total cholesterol is at least about 30-40%; (ii) capable of reducing serum LDL cholesterol at least about 65-80% and sustaining the reduction over at least a 24 day period relative to a predose level; (iii) capable of reducing serum triglyceride at least about 25-40% relative to predose level; (iv) does not reduce serum HDL cholesterol or reduces serum HDL cholesterol no more than 5% relative to predose level; (v) binds an epitope comprising amino acid residue 238 of hPCSK9 (SEQ ID NO:755); (vi) exhibits an enhanced binding affinity (K_{D}) for hPCSK9 at pH 5.5 relative to the K_{D} at pH 7.4, as measured by plasmon surface resonance, wherein the enhanced affinity is at least about a 20-to 50-fold increase in affinity; (vii) binds human, human GOF mutation D374Y, cynomolgus monkey, rhesus monkey, mouse, rat and hamster PCSK9; (viii) comprises heavy and light chain CDR3 sequences comprising SEQ ID NO:80 and 88; and (ix) comprises CDR sequences from SEQ ID NO:90 and 92.

In one embodiment, the invention provides fully human monoclonal antibody or antigen-binding fragment thereof that specifically bind human PCSK9 (hPCSK9) and neutralize PCSK9 activity, wherein the antibody or fragment thereof exhibits one or more of the following characteristics: (i) capable of reducing serum LDL cholesterol at least about 40-70% and sustaining the reduction over at least a 60 or 90 day period relative to a predose level; (ii) capable of reducing serum triglyceride at least about 25-40% relative to predose level; (iii) does not reduce serum HDL cholesterol or reduces serum HDL cholesterol no more than 5% relative to predose level; (iv) binds an epitope comprising amino acid residue 366 of hPCSK9 (SEQ ID NO:755); (v) does not exhibit an enhanced binding affinity for PCSK9 at an acidic pH relative to a neutral pH, as measured by surface plasmon resonance; (vi) binds human and monkey PCSK9, but does not bind mouse, rat or hamster PCSK9; (vii) comprises heavy and light chain CDR3 sequences comprising SEQ ID NO:224 and 232; and (viii) comprises CDR sequences from SEQ ID NO:218 and 226.

In a third aspect, the invention provides nucleic acid molecules encoding anti-PCSK9 antibodies or fragments thereof. Recombinant expression vectors carrying the nucleic acids of the invention, and host cells into which such vectors have been introduced, are also encompassed by the invention, as are methods of producing the antibodies by culturing the host cells under conditions permitting production of the antibodies, and recovering the antibodies produced.

In one embodiment, the invention provides an antibody or fragment thereof comprising a HCVR encoded by a nucleic acid sequence selected from the group consisting of SEQ ID NO: 1, 17, 21, 25, 41, 45, 49, 65, 69, 73, 89, 93, 97, 113, 117, 121, 137, 141, 145, 161, 165, 169, 185, 189, 193, 209, 213, 217, 233, 237, 241, 257, 261, 265, 281, 285, 289, 305, 309, 313, 329, 333, 337, 353, 357, 361, 377, 381, 385, 401, 405, 409, 425, 429, 433, 449, 453, 457, 473, 477, 481, 497, 501, 505, 521, 525, 529, 545, 549, 553, 569, 573, 577, 593, 597, 601, 617, 621, 625, 641, 645, 649, 665, 669, 673, 689, 693, 697, 713, 717, 721, 737 and 741, or a substantially identical sequence having at least 90%, at least 95%, at least 98%, or at least 99% homology thereof. In one embodiment, the HCVR is encoded by a nucleic acid sequence selected from the group consisting of SEQ ID NO: 49, 65, 69, 73, 89, 93, 121, 137, 141, 217, 233, 237, 241, 257, 261, 313, 329 and 333. In more specific embodiments, the HCVR is encoded by a nucleic acid sequence selected from the group consisting of SEQ ID NO: 89 and 217.

In one embodiment, the antibody or fragment thereof further comprises a LCVR encoded by a nucleic acid sequence selected from the group consisting of SEQ ID NO: 9, 19, 23, 33, 43, 47, 57, 67, 71, 81, 91, 95, 105, 115, 119, 129, 139, 143, 153, 163, 167, 177, 187, 191, 201, 211, 215, 225, 235, 239, 249, 259, 263, 273, 283, 287, 297, 307, 311, 321, 331, 335, 345, 355, 359, 369, 379, 383, 393, 403, 407, 417, 427, 431, 441, 451, 455, 465, 475, 479, 489, 499, 503, 513, 523, 527, 537, 547, 551, 561, 571, 575, 585, 595, 599, 609, 619, 623, 633, 643, 647, 657, 667, 671, 681, 691, 695, 705, 715, 719, 729, 739 and 743, or a substantially identical sequence having at least 90%, at least 95%, at least 98%, or at least 99% homology thereof. In one embodiment, the LCVR is encoded by a nucleic acid sequence selected from the group consisting of SEQ ID NO: 57, 67, 71, 81, 91, 95, 129, 139, 143, 225, 235, 239, 249, 259, 263, 321, 331 and 335. In more specific embodiments, the LCVR is encoded by a nucleic acid sequence selected from the group consisting of SEQ ID NO: 91 and 225.

In one embodiment, the invention features an antibody or antigen-binding fragment of an antibody comprising a HCDR3 domain encoded by a nucleotide sequence selected from the group consisting of SEQ ID NO:7, 31, 55, 79, 103, 127, 151, 175, 199, 223, 247, 271, 295, 319, 343, 367, 391, 415, 439, 463, 487, 511, 535, 559, 583, 607, 631, 655, 679, 703 and 727, or a substantially identical sequence having at least 90%, at least 95%, at least 98%, or at least 99% homology thereof; and a LCDR3 domain encoded by a nucleotide sequence selected from the group consisting of SEQ ID NO: 15, 39, 63, 87, 111, 135, 159, 183, 207, 231, 255, 279, 303, 327, 351, 375, 399, 423, 447, 471, 495, 519, 543, 567, 591, 615, 639, 663, 687, 711 and 735, or a substantially identical sequence having at least 90%, at least 95%, at least 98%, or at least 99% homology thereof. In one embodiment, the HCDR3 and LCDR3 comprise a sequence pair encoded by the nucleic acid sequence of SEQ ID NO: 55/63, 79/87, 127/135, 223/231, 247/255 and 319/327, respectively. In more specific embodiments, the HCDR3 and LCDR3 comprise a sequence pair encoded by the nucleic acid sequence of SEQ ID NO: 79/87 and 223/231.

In a further embodiment, the antibody or fragment thereof further comprises, a HCDR1 domain encoded by a nucleotide sequence selected from the group consisting of SEQ ID NO: 3, 27, 51, 75, 99, 123, 147, 171, 195, 219, 243, 267, 291, 315, 339, 363, 387, 411, 435, 459, 483, 507, 531, 555, 579, 603, 627, 651, 675, 699 and 723, or a substantially identical sequence having at least 90%, at least 95%, at least 98%, or at least 99% homology thereof; a HCDR2 domain encoded by a nucleotide sequence selected from the group consisting of SEQ ID NO:5, 29, 53, 77, 101, 125, 149, 173, 197, 221, 245, 269, 293, 317, 341, 365, 389, 413, 437, 461, 485, 509, 533, 557, 581, 605, 629, 653, 677, 701 and 725, or a substantially identical sequence having at least 90%, at least 95%, at least 98%, or at least 99% homology thereof; a LCDR1 domain encoded by a nucleotide sequence selected from the group consisting of SEQ ID NO: 11, 35, 59, 83, 107, 131, 155, 179, 203, 227, 251, 275, 299, 323, 347, 371, 395, 419, 443, 467, 491, 515, 539, 563, 587, 611, 635, 659, 683, 707 and 731, or a substantially identical sequence having at least 90%, at least 95%, at least 98%, or at least 99% homology thereof; and a LCDR2 domain encoded by a nucleotide sequence selected from the group consisting of SEQ ID NO: 13, 37, 61, 85, 109, 133, 157, 181, 205, 229, 253, 277, 301, 325, 349, 373, 397, 421, 445, 469, 493, 517, 541, 565, 589, 613, 637, 661, 685, 709 and 733, or a substantially identical sequence having at least 90%, at least 95%, at least 98%, or at least 99% homology thereof. In one embodiment, the heavy and light chain CDR sequences are encoded by the nucleic acid sequences of SEQ ID NO: 51, 53, 55, 59, 61, 63; 75, 77, 79, 83, 85, 87; 123, 125, 127, 131, 133, 135; 219, 221, 223, 227, 229, 231; 243, 245, 247, 251, 253, 255; and 315, 317, 319, 323, 325, 327. In more specific embodiments, the heavy and light chain CDR sequences are encoded by the nucleic acid sequences of SEQ ID NO: 75, 77, 79, 83, 85, 87; and 219, 221, 223, 227, 229, 231.

In a fourth aspect, the invention features an isolated antibody or antigen-binding fragment thereof that specifically binds hPCSK9, comprising a HCDR3 and a LCDR3, wherein HCDR3 comprises an amino acid sequence of the formula X¹-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸-X¹⁹-X²⁰ (SEQ ID NO: 747), wherein X¹ is Ala, X² is Arg or Lys, X³ is Asp, X⁴ is Ser or Ile, X⁵ is Asn or Val, X⁶ is Leu or Trp, X⁷ is Gly or Met, X⁸ is Asn or Val, X⁹ is Phe or Tyr, X¹⁰ is Asp, X¹¹ is Leu or Met, X¹² is Asp or absent, X¹³ is Tyr or absent, X¹⁴ is Tyr or absent, X¹⁵ is Tyr or absent, X¹⁶ is Tyr or absent, X¹⁷ is Gly or absent, X¹⁸ is Met or absent, X¹⁹ is Asp or absent, and X²⁰ is Val or absent; and LCDR3 comprises an amino acid sequence of the formula X¹-X²-X³-X⁴-X⁵-X⁶-X⁷-X⁸-X⁹ (SEQ ID NO:750), wherein X¹ is Gln or Met, X² is Gln, X³ is Tyr or Thr, X⁴ is Tyr or Leu, X⁵ is Thr or Gln, X⁶ is Thr, X⁷ is Pro, X⁸ is Tyr or Leu, and X⁹ is Thr.

In a further embodiment, the antibody or fragment thereof further comprise a HCDR1 sequence of the formula X¹-X²-X³-X⁴-X⁵-X⁶-X⁷-X⁸ (SEQ ID NO:745), wherein X¹ is Gly, X² is Phe, X³ is Thr, X⁴ is Phe, X⁵ is Ser or Asn, X⁶ is Ser or Asn, X⁷ is Tyr or His, and X⁸ is Ala or Trp; a HCDR2 sequence of the formula X¹-X²-X³-X⁴-X⁵-X⁶-X⁷-X⁸ (SEQ ID NO: 746), wherein X¹ is Ile, X² is Ser or Asn, X³ is Gly or Gin, X⁴ is Asp or Ser, X⁵ is Gly, X⁶ is Ser or Gly, X⁷ is Thr or Glu, and X⁸ is Thr or Lys; a LCDR1 sequence of the formula X¹-X²-X³-X⁴-X⁵-X⁶-X⁷-X⁸-X⁹-X¹⁰-X¹¹-X¹² (SEQ ID NO:748) wherein X¹ is Gln, X² is Ser, X³ is Val or Leu, X⁴ is Leu, X⁵ is His or Tyr, X⁶ is Arg or Ser, X⁷ is Ser or Asn, X⁸ is Asn or Gly, X⁹ is Asn, X¹⁰ is Arg or Asn, X¹¹ is Asn or Tyr, and X¹² is Phe or absent; a LCDR2 sequence of the formula X¹-X²-X³ (SEQ ID NO:749) wherein X¹ is Trp or Leu, X² is Ala or Gly, and X³ is Ser. FIG. 1 shows the sequence alignment of heavy and light chain variable regions for 316P and 300N mAbs.

In a fifth aspect, the invention features a human anti-PCSK9 antibody or antigen-binding fragment of an antibody comprising a heavy chain variable region (HCVR) encoded by nucleotide sequence segments derived from V_{H}, D_{H} and J_{H} germline sequences, and a light chain variable region (LCVR) encoded by nucleotide sequence segments derived from V_{K} and J_{K} germline sequences, wherein the germline sequences are (a) V_{H} gene segment 3-23, D_{H} gene segment 7-27, J_{H} gene segment 2, V_{K} gene segment 4-1 and J_{K} gene segment 2; or (b) V_{H} gene segment 3-7, D_{H} gene segment 2-8, J_{H} gene segment 6, V_{K} gene segment 2-28 and J_{K} gene segment 4.

In a sixth aspect, the invention features an antibody or antigen-binding fragment thereof that binds to a PCSK9 protein of SEQ ID NO:755, wherein the binding of the antibody or fragment thereof to a variant PCSK9 protein is less than 50% of the binding between the antibody or fragment thereof and the PCSK9 protein of SEQ ID NO:755. In specific embodiment, the antibody or fragment thereof binds to the variant PCSK9 protein with a binding affinity (K_{D}) which is less than about 50%, less than about 60%, less than about 70%, less than about 80%, less than about 90% or less than about 95% compared to the binding to PCSK9 (SEQ ID NO:755).

In one embodiment, the variant PCSK9 protein comprises at least one mutation at position 238 of SEQ ID NO:755. In a more specific embodiment, the mutation is D238R. In one embodiment, the antibody or antibody fragment binding affinity for the variant PCSK9 protein is at least 90% less relative to the wildtype protein of SEQ ID NO:755, wherein the variant protein comprises a mutation at residue 238. In one embodiment, the antibody or antibody fragment binding affinity for the variant PCSK9 protein is at least 80% less relative to the wildtype protein of SEQ ID NO:755, wherein the variant protein comprises a mutation at one or more of residue 153, 159, 238 and 343. In a more specific embodiment, the mutation is one of S153R, E159R, D238R and D343R.

In one embodiment, the variant PCSK9 protein comprises at least one mutation at position 366 of SEQ ID NO:755. In a more specific embodiment, the mutation is E366K. In one embodiment, the antibody or antibody fragment binding affinity for the variant PCSK9 protein is at least 95% less relative to the wildtype protein of SEQ ID NO:755, wherein the variant protein comprises a mutation at residue 366. In one embodiment, the antibody or antibody fragment binding affinity for the variant PCSK9 protein is at least 90% less relative to the wildtype protein of SEQ ID NO:755, wherein the variant protein comprises a mutation at one or more of residue 147, 366 and 380. In a more specific embodiment, the mutation is one of S147F, E366K and V380M. In one embodiment, the antibody or antibody fragment binding affinity for the variant PCSK9 protein is at least 80% less relative to the wildtype protein of SEQ ID NO:755, wherein the variant protein comprises a mutation at one or more of residue 147, 366 and 380. In a more specific embodiment, the mutation is one of S147F, E366K and V380M.

The invention encompasses anti-PCSK9 antibodies having a modified glycosylation pattern. In some applications, modification to remove undesirable glycosylation sites may be useful, or e.g., removal of a fucose moiety to increase antibody dependent cellular cytotoxicity (ADCC) function (see Shield et al. (2002) JBC 277:26733). In other applications, modification of galactosylation can be made in order to modify complement dependent cytotoxicity (CDC).

In a seventh aspect, the invention features a pharmaceutical composition comprising a recombinant human antibody or fragment thereof that specifically binds hPCSK9 and a pharmaceutically acceptable carrier. In one embodiment, the invention features a composition that is a combination of an antibody or antigen-binding fragment of an antibody of the invention, and a second therapeutic agent. The second therapeutic agent may be any agent that is advantageously combined with the antibody or fragment thereof of the invention, for example, an agent capable of inducing a cellular depletion of cholesterol synthesis by inhibiting 3-hydroxy-3-methylglutaryl (HMG)-coenzyme A (CoA) reductase, such as, for example, cerovastatin, atorvastatin, simvastatin, pitavastin, rosuvastatin, fluvastatin, lovastatin, pravastatin, etc.; capable of inhibiting cholesterol uptake and or bile acid re-absorption; capable of increasing lipoprotein catabolismsuch as niacin); and/or activators of the LXR transcription factor that plays a role in cholesterol elimination such as 22-hydroxycholesterol.

In an eighth aspect, the invention features methods for inhibiting hPCSK9 activity using the anti-PCSK9 antibody or antigen-binding portion of the antibody of the invention, wherein the therapeutic methods comprise administering a therapeutically effective amount of a pharmaceutical composition comprising an antibody or antigen-binding fragment of an antibody of the invention. The disorder treated is any disease or condition that is improved, ameliorated, inhibited or prevented by removal, inhibition or reduction of PCSK9 activity. Specific populations treatable by the therapeutic methods of the invention include subjects indicated for LDL apheresis, subjects with PCSK9-activating mutations (gain of function mutations, “GOF”), subjects with heterozygous Familial Hypercholesterolemia (heFH); subjects with primary hypercholesterolemia who are statin intolerant or statin uncontrolled; and subjects at risk for developing hypercholesterolemia who may be preventably treated. Other indications include dyslipidemia associated with secondary causes such as Type 2 diabetes mellitus, cholestatic liver diseases (primary biliary cirrhosis), nephrotic syndrome, hypothyroidism, obesity; and the prevention and treatment of atherosclerosis and cardiovascular diseases.

In specific embodiments of the method of the invention, the anti-hPCSK9 antibody or antibody fragment of the invention is useful to reduce elevated total cholesterol, non-HDL cholesterol, LDL cholesterol, and/or apolipoprotein B (apolipoprotein B100).

The antibody or antigen-binding fragment of the invention may be used alone or in combination with a second agent, for example, an HMG-CoA reductase inhibitor and/or other lipid lowering drugs.

Other embodiments will become apparent from a review of the ensuing detailed description.

### BRIEF DESCRIPTION OF THE FIGURES

FIG. 1. Sequence comparison tables of heavy chain (A) and light chain (B) variable regions and CDRs of antibodies H1H316P and H1M300N.

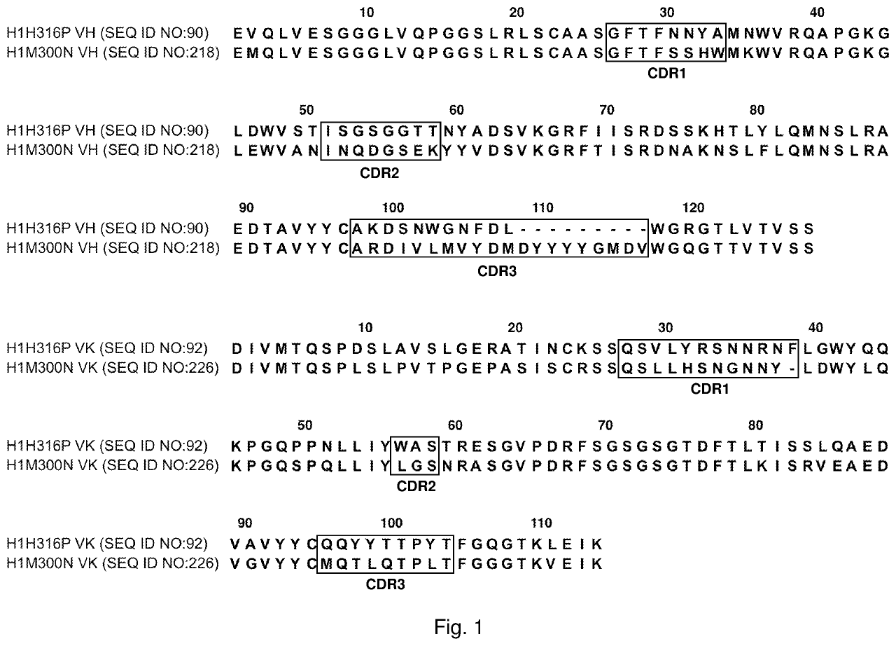

FIG. 2. Antibody concentrations in serum over time. 316P 5 mg/kg (□); 300N 5 mg/kg (∘); 316P 15 mg/kg (▮) 300N 15 mg/kg (●).

FIG. 3. Serum total cholesterol level as a percentage of change over buffer control. Buffer control ( [symbol] ) 316P 5 mg/kg (▮) 300N 5 mg/kg (▴); 316P 15 mg/kg (□); 300N 15 mg/kg (Δ).

FIG. 4. Serum LDL cholesterol level as a percentage of change over buffer control: Buffer Control ( [symbol] ); 316P 5 mg/kg (▮) 300N 5 mg/kg (▴); 316P 15 mg/kg (□); 300N 15 mg/kg (Δ).

FIG. 5. Serum LDL cholesterol level normalized to buffer control. Buffer control ( [symbol] ); 316P 5 mg/kg (▮) 300N 5 mg/kg (▴); 316P 15 mg/kg (□); 300N 15 mg/kg (Δ).

FIG. 6. Serum HDL cholesterol level as a percentage of change over buffer control. Buffer control ( [symbol] ); 316P 5 mg/kg (▮) 300N 5 mg/kg (▴); 316P 15 mg/kg (□); 300N 15 mg/kg (Δ).

FIG. 7. Serum triglyceride level expressed as a percentage of change over buffer control. Buffer control ( [symbol] ); 316P 5 mg/kg (▮) 300N 5 mg/kg (▴); 316P 15 mg/kg (□); 300N 15 mg/kg (Δ).

FIG. 8. Serum LDL cholesterol level expressed as a percentage of change over baseline following a single dose subcutaneous administration. 316P 5 mg/kg (▮) 300N 5 mg/kg (●).

FIG. 9. Antibody concentrations in serum over time following a single dose subcutaneous administration. 316P 5 mg/kg (●); 300N 5 mg/kg (▴).

FIG. 10. Western blot for mouse LDL receptor of total liver homogenates. Samples were taken 24 hours after PBS (lanes 1-3), 5 mg/kg 316P (lanes 4-6), or 5 mg/kg of non-hPCSK9 specific mAb (lanes 7-8) administration and 4 hours after 1.2 mg/kg hPCSK9-mmh (all lanes).

FIG. 11. Effects of 316P on serum LDL cholesterol level in PCSKg^{hu/hu} mice. Buffer [symbol] control 316P 1 mg/kg ( [symbol] ); 316P 5 mg/kg ( [symbol] ); 316P 10 mg/kg ( [symbol] ).

FIG. 12. Anti-hPCSK9 mAb serum pharmacokinetic profile in C57BL/6 mice. Single dose of Control I mAb (●) at 10 mg/kg; 316P (▴) at 10 mg/kg and 300N (▮) at 10 mg/kg.

FIG. 13. Anti-hPCSK9 mAb serum pharmacokinetic profile in hPCSK9 heterozygous mice. Single dose of Control I mAb (●) at 10 mg/kg; 316P (▴) at 10 mg/kg and 300N (▮) at 10 mg/kg.

FIG. 14. Effect of 316P on serum LDL cholesterol levels in Syrian Hamster fed a normal diet. Buffer control (●) 316P 1 mg/kg (▮) 316P 3 mg/kg (▴); 316P 5 mg/kg (▾).

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, preferred methods and materials are now described. All publications mentioned herein are incorporated herein by reference in their entirety.

### Definitions

The term “human proprotein convertase subtilisin/kexin type 9” or “hPCSK9”, as used herein, refers to hPCSK9 having the nucleic acid sequence shown in SEQ ID NO:754 and the amino acid sequence of SEQ ID NO:755, or a biologically active fragment thereof.

The term “antibody”, as used herein, is intended to refer to immunoglobulin molecules comprised of four polypeptide chains, two heavy (H) chains and two light (L) chains inter-connected by disulfide bonds. Each heavy chain is comprised of a heavy chain variable region (“HCVR” or “VH”) and a heavy chain constant region (comprised of domains CH1, CH2 and CH3). Each light chain is comprised of a light chain variable region (“LCVR or “VL”) and a light chain constant region (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

Substitution of one or more CDR residues or omission of one or more CDRs is also possible. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The term “human antibody”, as used herein, is intended to include antibodies having variable and constant regions derived from human germline immunoglobulin sequences. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse), have been grafted onto human FR sequences.

The term “specifically binds,” or the like, means that an antibody or antigen-binding fragment thereof forms a complex with an antigen that is relatively stable under physiologic conditions. Specific binding can be characterized by an equilibrium dissociation constant of at least about 1×10⁻⁶ M or less (e.g., a smaller K_{D} denotes a tighter binding). Methods for determining whether two molecules specifically bind are well known in the art and include, for example, equilibrium dialysis, surface plasmon resonance, and the like. An isolated antibody that specifically binds hPCSK9 may, however, exhibit cross-reactivity to other antigens such as PCSK9 molecules from other species. Moreover, multi-specific antibodies (e.g., bispecifics) that bind to hPCSK9 and one or more additional antigens are nonetheless considered antibodies that “specifically bind” hPCSK9, as used herein.

The term “high affinity” antibody refers to those mAbs having a binding affinity to hPCSK9 of at least 10⁻¹⁰ M; preferably 10⁻¹¹ M; even more preferably 10⁻¹² M, as measured by surface plasmon resonance, e.g., BIACORE™ or solution-affinity ELISA.

By the term “slow off rate”, “Koff” or “kd” is meant an antibody that dissociates from hPCSK9 with a rate constant of 1×10⁻³ s⁻¹ or less, preferably 1×10⁻⁴ s⁻¹ or less, as determined by surface plasmon resonance, e.g., BIACORE™.

The term “antigen-binding portion” of an antibody (or simply “antibody fragment”), as used herein, refers to one or more fragments of an antibody that retain the ability to specifically bind to hPCSK9. An antibody fragment may include a Fab fragment, a F(ab′)₂ fragment, a Fv fragment, a dAb fragment, a fragment containing a CDR, or an isolated CDR.

The specific embodiments, antibody or antibody fragments of the invention may be conjugated to a therapeutic moiety (“immunoconjugate”), such as a cytotoxin, a chemotherapeutic drug, an immunosuppressant or a radioisotope.

An “isolated antibody”, as used herein, is intended to refer to an antibody that is substantially free of other mAbs having different antigenic specificities (e.g., an isolated antibody that specifically binds hPCSK9 is substantially free of mAbs that specifically bind antigens other than hPCSK9). An isolated antibody that specifically binds hPCSK9 may, however, have cross-reactivity to other antigens, such as PCSK9 molecules from other species.

A “neutralizing antibody”, as used herein (or an “antibody that neutralizes PCSK9 activity”), is intended to refer to an antibody whose binding to hPCSK9 results in inhibition of at least one biological activity of PCSK9. This inhibition of the biological activity of PCSK9 can be assessed by measuring one or more indicators of PCSK9 biological activity by one or more of several standard in vitro or in vivo assays known in the art (see examples below).

The term “surface plasmon resonance”, as used herein, refers to an optical phenomenon that allows for the analysis of real-time biospecific interactions by detection of alterations in protein concentrations within a biosensor matrix, for example using the BIACORE™ system (Pharmacia Biosensor AB, Uppsala, Sweden and Piscataway, N.J.).

The term “K_{D}”, as used herein, is intended to refer to the equilibrium dissociation constant of a particular antibody-antigen interaction.

The term “epitope” is a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions that are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24:307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT, which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403 410 and (1997) Nucleic Acids Res. 25:3389 402, each of which is herein incorporated by reference.

In specific embodiments, the antibody or antibody fragment for use in the method of the invention may be monospecific, bispecific, or multispecific. Multispecific antibodies may be specific for different epitopes of one target polypeptide or may contain antigen-binding domains specific for epitopes of more than one target polypeptide. An exemplary bi-specific antibody format that can be used in the context of the present invention involves the use of a first immunoglobulin (Ig) CH3 domain and a second Ig CH3 domain, wherein the first and second Ig CH3 domains differ from one another by at least one amino acid, and wherein at least one amino acid difference reduces binding of the bispecific antibody to Protein A as compared to a bi-specific antibody lacking the amino acid difference. In one embodiment, the first Ig CH3 domain binds Protein A and the second Ig CH3 domain contains a mutation that reduces or abolishes Protein A binding such as an H95R modification (by IMGT exon numbering; H435R by EU numbering). The second CH3 may further comprise an Y96F modification (by IMGT; Y436F by EU). Further modifications that may be found within the second CH3 include: D16E, L18M, N44S, K52N, V57M, and V821 (by IMGT; D356E, L358M, N384S, K392N, V397M, and V4221 by EU) in the case of IgG1 mAbs; N44S, K52N, and V821 (IMGT; N384S, K392N, and V4221 by EU) in the case of IgG2 mAbs; and Q15R, N44S, K52N, V57M, R69K, E79Q, and V821 (by IMGT; Q355R, N384S, K392N, V397M, R409K, E419Q, and V4221 by EU) in the case of IgG4 mAbs. Variations on the bi-specific antibody format described above are contemplated within the scope of the present invention.

By the phrase “therapeutically effective amount” is meant an amount that produces the desired effect for which it is administered. The exact amount will depend on the purpose of the treatment, and will be ascertainable by one skilled in the art using known techniques (see, for example, Lloyd (1999) The Art, Science and Technology of Pharmaceutical Compounding).

### Preparation of Human Antibodies

Methods for generating human antibodies in transgenic mice are known (see for example, U.S. Pat. No. 6,596,541, Regeneron Pharmaceuticals, VELOCIMMUNE™). The VELOCIMMUNE™ technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody. In specific embodiment, the cell is a CHO cell.

Antibodies may be therapeutically useful in blocking a ligand-receptor interaction or inhibiting receptor component interaction, rather than by killing cells through fixation of complement and participation in complement-dependent cytotoxicity (CDC), or killing cells through antibody-dependent cell-mediated cytotoxicity (ADCC). The constant region of an antibody is thus important in the ability of an antibody to fix complement and mediate cell-dependent cytotoxicity. Thus, the isotype of an antibody may be selected on the basis of whether it is desirable for the antibody to mediate cytotoxicity.

Human antibodies can exist in two forms that are associated with hinge heterogeneity. In one form, an antibody molecule comprises a stable four-chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant invention encompasses antibodies having one or more mutations in the hinge, CH2, or CH3 region, which may be desirable, for example, in production, to improve the yield of the desired antibody form.

Generally, a VELOCIMMUNE™ mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. As described below, the antibodies are characterized and selected for desirable characteristics, including affinity, selectivity, epitope, etc. The mouse constant regions are replaced with a desired human constant region to generate the fully human antibody of the invention, for example wild-type or modified IgG1 or IgG4 (for example, SEQ ID NO:751, 752, 753). While the constant region selected may vary according to specific use, high affinity antigen binding and target specificity characteristics reside in the variable region.

### Epitope Mapping and Related Technologies

To screen for antibodies that bind to a particular epitope (e.g., those which block binding of IgE to its high affinity receptor), a routine cross-blocking assay such as that described Antibodies, Harlow and Lane (Cold Spring Harbor Press, Cold Spring Harb., NY) can be performed. Other methods include alanine scanning mutants, peptide blots (Reineke (2004) Methods Mol Biol 248:443-63) (herein specifically incorporated by reference in its entirety), or peptide cleavage analysis. In addition, methods such as epitope excision, epitope extraction and chemical modification of antigens can be employed (Tomer (2000) Protein Science 9: 487-496) (herein specifically incorporated by reference in its entirety).

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (US 2004/0101920, herein specifically incorporated by reference in its entirety). Each category may reflect a unique epitope either distinctly different from or partially overlapping with epitope represented by another category. This technology allows rapid filtering of genetically identical mAbs, such that characterization can be focused on genetically distinct mAbs. When applied to hybridoma screening, MAP may facilitate identification of rare hybridoma clones that produce mAbs having the desired characteristics. MAP may be used to sort the anti-PCSK9 mAbs of the invention into groups of mAbs binding different epitopes.

In various embodiments, the anti-hPCSK9 antibody or antigen-binding fragment of an antibody binds an epitope within the catalytic domain, which is about 153 to 425 of SEQ ID NO: 755); more specifically, an epitope from about 153 to about 250 or from about 250 to about 425; more specifically, the antibody or antibody fragment of the invention binds an epitope within the fragment from about 153 to about 208, from about 200 to about 260, from about 250 to about 300, from about 275 to about 325, from about 300 to about 360, from about 350 to about 400, and/or from about 375 to about 425.

In various embodiments, the anti-hPCSK9 antibody or antigen-binding fragment of an antibody binds an epitope within the propeptide domain (residues 31 to 152 of SEQ ID NO: 755); more specifically, an epitope from about residue 31 to about residue 90 or from about residue 90 to about residue 152; more specifically, the antibody or antibody fragment of the invention binds an epitope within the fragment from about residue 31 to about residue 60, from about residue 60 to about residue 90, from about residue 85 to about residue 110, from about residue 100 to about residue 130, from about residue 125 to about residue 150, from about residue 135 to about residue 152, and/or from about residue 140 to about residue 152.

In some embodiments, the anti-hPCSK9 antibody or antigen-binding fragment of an antibody binds an epitope within the C-terminal domain, (residues 426 to 692 of SEQ ID NO: 755); more specifically, an epitope from about residue 426 to about residue 570 or from about residue 570 to about residue 692; more specifically, the antibody or antibody fragment of the invention binds an epitope within the fragment from about residue 450 to about residue 500, from about residue 500 to about residue 550, from about residue 550 to about residue 600, and/or from about residue 600 to about residue 692.

In some embodiments, the antibody or antibody fragment binds an epitope which includes more than one of the enumerated epitopes within the catalytic, propeptide or C-terminal domain, and/or within two or three different domains (for example, epitopes within the catalytic and C-terminal domains, or within the propeptide and catalytic domains, or within the propeptide, catalytic and C-terminal domains.

In some embodiments, the antibody or antigen-binding fragment binds an epitope on hPCSK9 comprising amino acid residue 238 of hPCSK9 (SEQ ID NO:755). Experimental results (Table 27) show that when D238 was mutated, the K_{D} of mAb 316P exhibited >400-fold reduction in binding affinity (˜1×10⁻⁹ M to ˜410×10⁻⁹ M) and T_{1/2} decreased >30-fold (from ˜37 to ˜1 min). In a specific embodiment, the mutation was D238R. In specific embodiments, the antibody or antigen-binding fragment of the invention binds an epitope of hPCSK9 comprising two or more of amino acid residues at positions 153, 159, 238 and 343.

As shown below, a mutation in amino acid residue 153, 159 or 343 resulted in about a 5- to 10-fold decrease in affinity or similar shortening in T_{1/2}. In specific embodiments, the mutation was S153R, E159R and/or D343R.

In some embodiments, the antibody or antigen-binding fragment binds an epitope on hPCSK9 comprising amino acid residue 366 of hPCSK9 (SEQ ID NO:755). Experimental results (Table 27) show that when E366 was mutated, the affinity of mAb 300N exhibited about 50-fold decrease (˜0.7×10⁻⁹ M to ˜36×10⁻⁹ M) and a similar shortening in T_{1/2} (from ˜120 to ˜2 min). In a specific embodiment, the mutation is E366K.

The present invention includes anti-PCSK9 antibodies that bind to the same epitope as any of the specific exemplary antibodies described herein. Likewise, the present invention also includes anti-PCSK9 antibodies that compete for binding to PCSK9 or a PCSK9 fragment with any of the specific exemplary antibodies described herein.

One can easily determine whether an antibody binds to the same epitope as, or competes for binding with, a reference anti-PCSK9 antibody by using routine methods known in the art. For example, to determine if a test antibody binds to the same epitope as a reference anti-PCSK9 antibody of the invention, the reference antibody is allowed to bind to a PCSK9 protein or peptide under saturating conditions. Next, the ability of a test antibody to bind to the PCSK9 molecule is assessed. If the test antibody is able to bind to PCSK9 following saturation binding with the reference anti-PCSK9 antibody, it can be concluded that the test antibody binds to a different epitope than the reference anti-PCSK9 antibody. On the other hand, if the test antibody is not able to bind to the PCSK9 molecule following saturation binding with the reference anti-PCSK9 antibody, then the test antibody may bind to the same epitope as the epitope bound by the reference anti-PCSK9 antibody of the invention.

To determine if an antibody competes for binding with a reference anti-PCSK9 antibody, the above-described binding methodology is performed in two orientations: In a first orientation, the reference antibody is allowed to bind to a PCSK9 molecule under saturating conditions followed by assessment of binding of the test antibody to the PCSK9 molecule. In a second orientation, the test antibody is allowed to bind to a PCSK9 molecule under saturating conditions followed by assessment of binding of the reference antibody to the PCSK9 molecule. If, in both orientations, only the first (saturating) antibody is capable of binding to the PCSK9 molecule, then it is concluded that the test antibody and the reference antibody compete for binding to PCSK9. As will be appreciated by a person of ordinary skill in the art, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

In a specific embodiment, the invention comprises an anti-PCSK9 antibody or antigen binding fragment of an antibody that binds an PCSK9 protein of SEQ ID NO:755, wherein the binding between the antibody or fragment thereof to PCSK9 and a variant PCSK9 protein is less than 50% of the binding between the antibody or fragment and the PCSK9 protein of SEQ ID NO:755. In one specific embodiment, the variant PCSK9 protein comprises at least one mutation of a residue at a position selected from the group consisting of 153, 159, 238 and 343. In a more specific embodiment, the at least one mutation is S153R, E159R, D238R, and/or D343R. In another specific embodiment, the variant PCSK9 protein comprises at least one mutation of a residue at a position selected from the group consisting of 366. In one specific embodiment, the variant PCSK9 protein comprises at least one mutation of a residue at a position selected from the group consisting of 147, 366 and 380. In a more specific embodiment, the mutation is S147F, E366K and V380M.

### Immunoconjugates

The invention encompasses a human anti-PCSK9 monoclonal antibody conjugated to a therapeutic moiety (“immunoconjugate”), such as a cytotoxin, a chemotherapeutic drug, an immunosuppressant or a radioisotope. Cytotoxin agents include any agent that is detrimental to cells. Examples of suitable cytotoxin agents and chemotherapeutic agents for forming immunoconjugates are known in the art, see for example, WO 05/103081.

### Bispecifics

The antibodies of the present invention may be monospecific, bispecific, or multispecific. Multispecific mAbs may be specific for different epitopes of one target polypeptide or may contain antigen-binding domains specific for more than one target polypeptide. See, e.g., Tutt et al. (1991) J. Immunol. 147:60-69. The human anti-PCSK9 mAbs can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment, to produce a bispecific or a multispecific antibody with a second binding specificity.

An exemplary bi-specific antibody format that can be used in the context of the present invention involves the use of a first immunoglobulin (Ig) CH3 domain and a second Ig CH3 domain, wherein the first and second Ig CH3 domains differ from one another by at least one amino acid, and wherein at least one amino acid difference reduces binding of the bispecific antibody to Protein A as compared to a bi-specific antibody lacking the amino acid difference. In one embodiment, the first Ig CH3 domain binds Protein A and the second Ig CH3 domain contains a mutation that reduces or abolishes Protein A binding such as an H95R modification (by IMGT exon numbering; H435R by EU numbering). The second CH3 may further comprise a Y96F modification (by IMGT; Y436F by EU). Further modifications that may be found within the second CH3 include: D16E, L18M, N44S, K52N, V57M, and V82I (by IMGT; D356E, L358M, N384S, K392N, V397M, and V422I by EU) in the case of IgG1 antibodies; N44S, K52N, and V821 (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies. Variations on the bi-specific antibody format described above are contemplated within the scope of the present invention.

### Bioequivalents

The anti-PCSK9 antibodies and antibody fragments of the present invention encompass proteins having amino acid sequences that vary from those of the described mAbs, but that retain the ability to bind human PCSK9. Such variant mAbs and antibody fragments comprise one or more additions, deletions, or substitutions of amino acids when compared to parent sequence, but exhibit biological activity that is essentially equivalent to that of the described mAbs. Likewise, the anti-PCSK9 antibody-encoding DNA sequences of the present invention encompass sequences that comprise one or more additions, deletions, or substitutions of nucleotides when compared to the disclosed sequence, but that encode an anti-PCSK9 antibody or antibody fragment that is essentially bioequivalent to an anti-PCSK9 antibody or antibody fragment of the invention. Examples of such variant amino acid and DNA sequences are discussed above.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied. In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of anti-PCSK9 antibodies of the invention may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation.

### Treatment Population

The invention provides therapeutic methods for treating a human patient in need of a composition of the invention. While modifications in lifestyle and conventional drug treatment are often successful in reducing cholesterol levels, not all patients are able to achieve the recommended target cholesterol levels with such approaches. Various conditions, such as familial hypercholesterolemia (FH), appear to be resistant to lowering of LDL-C levels in spite of aggressive use of conventional therapy. Homozygous and heterozygous familial hypercholesterolemia (hoFH, heFH) is a condition associated with premature atherosclerotic vascular disease. However, patients diagnosed with hoFH are largely unresponsive to conventional drug therapy and have limited treatment options. Specifically, treatment with statins, which reduce LDL-C by inhibiting cholesterol synthesis and upregulating the hepatic LDL receptor, may have little effect in patients whose LDL receptors are non-existent or defective. A mean LDL-C reduction of only less than about 20% has been recently reported in patients with genotype-confirmed hoFH treated with the maximal dose of statins. The addition of ezetimibe 10 mg/day to this regimen resulted in a total reduction of LDL-C levels of 27%, which is still far from optimal. Likewise, many patients are statin non-responsive, poorly controlled with statin therapy, or cannot tolerate statin therapy; in general, these patients are unable to achieve cholesterol control with alternative treatments. There is a large unmet medical need for new treatments that can address the shortcomings of current treatment options.

Specific populations treatable by the therapeutic methods of the invention include patients indicated for LDL apheresis, subjects with PCSK9-activating (GOF) mutations, heterozygous Familial Hypercholesterolemia (heFH); subjects with primary hypercholesterolemia who are statin intolerant or statin uncontrolled; and subjects at risk for developing hypercholesterolemia who may be preventably treated.

### Therapeutic Administration and Formulations

The invention provides therapeutic compositions comprising the anti-PCSK9 antibodies or antigen-binding fragments thereof of the present invention. The administration of therapeutic compositions in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose may vary depending upon the age and the size of a subject to be administered, target disease, conditions, route of administration, and the like. When the antibody of the present invention is used for treating various conditions and diseases associated with PCSK9, including hypercholesterolemia, disorders associated with LDL and apolipoprotein B, and lipid metabolism disorders, and the like, in an adult patient, it is advantageous to intravenously administer the antibody of the present invention normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The pharmaceutical composition can be also delivered in a vesicle, in particular a liposome (see Langer (1990) Science 249:1527-1533; Treat et al. (1989) in Liposomes in the Therapy of Infectious Disease and Cancer, Lopez Berestein and Fidler (eds.), Liss, New York, pp. 353-365; Lopez-Berestein, ibid., pp. 317-327; see generally ibid.).

In certain situations, the pharmaceutical composition can be delivered in a controlled release system. In one embodiment, a pump may be used (see Langer, supra; Sefton (1987) CRC Crit. Ref. Biomed. Eng. 14:201). In another embodiment, polymeric materials can be used; see, Medical Applications of Controlled Release, Langer and Wise (eds.), CRC Pres., Boca Raton, Florida (1974). In yet another embodiment, a controlled release system can be placed in proximity of the composition's target, thus requiring only a fraction of the systemic dose (see, e.g., Goodson, in Medical Applications of Controlled Release, supra, vol. 2, pp. 115-138, 1984).

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule. A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™ and OPTICLIK™ (sanofi-aventis, Frankfurt, Germany), to name only a few. Examples of disposable pen delivery devices having applications in subcutaneous delivery of a pharmaceutical composition of the present invention include, but certainly are not limited to the SOLOSTAR™ pen (sanofi-aventis), the FLEXPEN™ (Novo Nordisk), and the KWIKPEN™ (Eli Lilly).

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

The invention provides therapeutic methods in which the antibody or antibody fragment of the invention is useful to treat hypercholesterolemia associated with a variety of conditions involving hPCSK9. The anti-PCSK9 antibodies or antibody fragments of the invention are particularly useful for the treatment of hypercholesterolemia and the like. Combination therapies may include the anti-PCSK9 antibody of the invention with, for example, one or more of any agent that (1) induces a cellular depletion of cholesterol synthesis by inhibiting 3-hydroxy-3-methylglutaryl (HMG)-coenzyme A (CoA) reductase, such as cerivastatin, atorvastatin, simvastatin, pitavastatin, rosuvastatin, fluvastatin, lovastatin, pravastatin; (2) inhibits cholesterol uptake and or bile acid re-absorption; (3) increase lipoprotein catabolismsuch as niacin); and activators of the LXR transcription factor that plays a role in cholesterol elimination such as 22-hydroxycholesterol or fixed combinations such as ezetimibe plus simvastatin; a statin with a bile resin (e.g., cholestyramine, colestipol, colesevelam), a fixed combination of niacin plus a statin (e.g., niacin with lovastatin); or with other lipid lowering agents such as omega-3-fatty acid ethyl esters (for example, omacor).

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used but some experimental errors and deviations should be accounted for. Unless indicated otherwise, molecular weight is average molecular weight, temperature is in degrees Centigrade, and pressure is at or near atmospheric.

### Example 1: Generation of Human Antibodies to Human PCSK9

VELOCIMMUNE™ mice were immunized with human PCSK9, and the antibody immune response monitored by antigen-specific immunoassay using serum obtained from these mice. Anti-hPCSK9 expressing B cells were harvested from the spleens of immunized mice shown to have elevated anti-hPCSK9 antibody titers were fused with mouse myeloma cells to form hybridomas. The hybridomas were screened and selected to identify cell lines expressing hPCSK9-specific antibodies using assays as described below. The assays identified several cell lines that produced chimeric anti-hPCSK9 antibodies designated as H1M300, H1M504, H1M505, H1M500, H1M497, H1M498, H1M494, H1M309, H1M312, H1M499, H1M493, H1M496, H1M503, H1M502, H1M508, H1M495 and H1M492.

Human PCSK9-specific antibodies were also isolated directly from antigen-immunized B cells without fusion to myeloma cells, as described in U.S. 2007/0280945A1, hereby incorporated by reference in its entirety. Heavy and light chain variable regions were cloned to generate fully human anti-hPCSK9 antibodies designated as H1H313, H1H314, H1H315, H1H316, H1H317, H1H318, H1H320, H1H321 and H1H334. Stable recombinant antibody-expressing CHO cell lines expressing these antibodies were established.

### Example 2. Gene Utilization Analysis

To analyze the structure of the mAbs produced, the nucleic acids encoding antibody variable regions were cloned and sequenced. The predicted amino acid sequences of the variable regions were confirmed by N-terminal amino acid sequencing. From the nucleic acid sequence and predicted amino acid sequence of the mAbs, gene usage was identified for each antibody chain.

TABLE 1

| | Heavy Chain | | | Light Chain | |
|---|---|---|---|---|---|
| | Variable | | | Variable | |
| | Region | | | Region | |
| Antibody | VH | D | JH | VK | JK |
| H1H313 | 3-13 | 1-26 | 4 | 3-15 | 3 |
| H1H314 | 3-33 | 3-3 | 4 | 1-5 | 2 |
| H1H315 | 3-33 | 3-3 | 4 | 4-1 | 1 |
| H1H316 | 3-23 | 7-27 | 2 | 4-1 | 2 |
| H1H317 | 3-13 | 1-26 | 4 | 1-6 | 1 |
| H1H318 | 4-59 | 3-10 | 6 | 1-9 | 1 |
| H1H320 | 1-18 | 2-2 | 6 | 2-30 | 1 |
| H1H321 | 2-5 | 1-7 | 6 | 2-28 | 4 |
| H1H334 | 2-5 | 6-6 | 6 | 2-28 | 4 |
| H1M300 | 3-7 | 2-8 | 6 | 2-28 | 4 |
| H1M504 | 3-30 | 2-8 | 6 | 2-28 | 4 |
| H1M505 | 3-30 | 2-8 | 6 | 2-28 | 4 |
| H1M500 | 2-5 | 5-5 | 6 | 2-28 | 4 |
| H1M497 | 1-18 | 2-2 | 6 | 2-30 | 2 |
| H1M498 | 3-21 | 2-2 | 4 | 1-5 | 2 |
| H1M494 | 3-11 | 5-12 | 6 | 3-20 | 4 |
| H1M309 | 3-21 | 6-13 | 4 | 1-5 | 1 |
| H1M312 | 3-21 | 6-13 | 4 | 1-5 | 1 |
| H1M499 | 3-21 | 6-13 | 4 | 1-5 | 1 |
| H1M493 | 3-21 | 6-13 | 4 | 1-5 | 1 |
| H1M496 | 3-13 | 6-19 | 4 | 3-15 | 3 |
| H1M503 | 1-18 | 2-2 | 6 | 2-28 | 1 |
| H1M502 | 3-13 | 6-13 | 4 | 3-15 | 3 |
| H1M508 | 3-13 | 6-13 | 4 | 3-15 | 3 |
| H1M495 | 3-9 | 4-17 | 6 | 1-9 | 3 |
| H1M492 | 3-23 | 3-3 | 2 | 3-20 | 4 |

### Example 3. Antigen Binding Affinity Determination

Equilibrium dissociation constants (K_{D}) for hPCSK9 binding to mAbs generated by hybridoma cell lines described above were determined by surface kinetics in a real-time biosensor surface plasmon resonance assay (BIACORE™ T100). Each antibody was captured at a flow rate of 4 μl/min for 90 sec on a goat anti-mouse IgG polyclonal antibody surface created through direct chemical coupling to a BIACORE™ chip to form a captured antibody surface. Human PCSK9-myc-myc-his (hPCSK9-mmh) at a concentration of 50 nM or 12.5 nM was injected over the captured antibody surfaces at a flowrate of 50 μl/min for 300 sec, and antigen-antibody dissociation was monitored for 15 min at either 25° C. or 37° C. (K_{D}=pM; T_{1/2}=min).

TABLE 2

| | 25° C. | | 37° C. | |
|---|---|---|---|---|
| Antibody | K_{D} | T_{1/2} | K_{D} | T_{1/2} |
| H1M300 | 399 | 170 | 1510 | 32 |
| H1M309 | 29.9 | 7461 | 537 | 326 |
| H1M312 | 0.225 | 15568 | 432 | 392 |
| H1M493 | 46.5 | 4921 | 522 | 341 |
| H1M494 | 870 | 114 | 2350 | 30 |
| H1M495 | 440 | 222 | 7500 | 19 |
| H1M496 | 254 | 257 | 421 | 118 |
| H1M497 | 20.1 | 5801 | 480 | 290 |
| H1M498 | 6400 | 30 | 7500 | 14 |
| H1M499 | 106 | 2253 | 582 | 316 |
| H1M500 | 1400 | 91 | 6010 | 15 |
| H1M502 | 78.3 | 958 | 411 | 151 |
| H1M503 | 510 | 118 | 1880 | 30 |
| H1M504 | 3470 | 35 | 11200 | 6 |
| H1M505 | 2740 | 42 | 9200 | 6 |
| H1M508 | 138 | 572 | 442 | 139 |
| H1M510 | 1070 | 68 | 3960 | 10 |

Equilibrium dissociation constants (K_{D}) for hPCSK9 binding to mAbs generated via direct isolation of splenocytes were determined by surface kinetics in a real-time biosensor surface plasmon resonance assay (BIACORE™ T100). Each selected antibody was captured at a flowrate of 2 μl/min for 6 min on a goat anti-human IgG polyclonal antibody surface created through direct chemical coupling to a BIACORE™ chip to form a captured antibody surface. Human PCSK9-mmh at a concentration of 50 nM or 12.5 nM was injected over the captured antibody surface at a flowrate of 70 μl/min for 5 min, and antigen-antibody dissociation was monitored for 15 min at either 25° C. or 37° C. (K_{D}=pM; T_{1/2}=min).

TABLE 3

| | 25° C. | | 37° C. | |
|---|---|---|---|---|
| Antibody | K_{D} | T_{1/2} | K_{D} | T_{1/2} |
| H1H313P | 244 | 230 | 780 | 60 |
| H1H314P | 3990 | 65 | 3560 | 43 |
| H1H315P | 129 | 151 | 413 | 35 |
| H1H316P | 377 | 42 | 1080 | 11 |
| H1H317P | 30400 | 137 | 18600 | 70 |
| H1H318P | 972 | 59 | 1690 | 28 |
| H1H320P | 771 | 28 | 1930 | 8 |
| H1H321P | 865 | 106 | 3360 | 23 |
| H1H334P | 3750 | 46 | 15900 | 8 |

Dissociation rate (kd) of selected mAbs for tagged rhesus monkey (Macaca mulata) PCSK9 (mmPCSK9; SEQ ID NO:756) (mmPCSK9-mmh) at 25° C. was determined as described above.

TABLE 4

| Antibody | kd (1/s) | T_{1/2} (min) |
|---|---|---|
| H1H313P | 2.92 × 10⁻⁵ | 396 |
| H1H318P | 3.69 × 10⁻³ | 3 |
| H1H334P | 8.06 × 10⁻³ | 1 |
| H1H315P | 2.29 × 10⁻⁴ | 51 |
| H1H316P | 2.29 × 10⁻⁴ | 51 |
| H1H320P | 3.17 × 10⁻⁴ | 36 |
| H1M300 | 1.52 × 10⁻⁴ | 76 |
| H1M504 | 5.04 × 10⁻⁴ | 23 |
| H1M497 | 6.60 × 10⁻⁵ | 175 |
| H1M503 | 8.73 × 10⁻⁵ | 132 |
| H1M496 | 4.45 × 10⁻⁵ | 260 |

### Example 4. Effect of pH on Antigen Binding Affinity

The effects of pH on antigen binding affinity for CHO cell-produced fully human anti-hPCSK9 mAbs was assessed as described above. The mAbs tested are fully human versions of H1H316P (“316P”) (HCVR/LCVR SEQ ID NO: 90/92; CDR sequences SEQ ID NO: 76/78/80 and 84/86/88) and H1M300N (“300N”) (HCVR/LCVR SEQ ID NO: 218/226; CDR sequences SEQ ID NO:220/222/224 and 228/232 and amino acid sequence LGS). Human PCSK9-myc-myc-his (hPCSK9-mmh) was captured on an anti-myc mAb surface either at a high density (about 35 to 45 resonance units) (RU) or at a low density (about 5 to 14 RU). Each antibody, at 50 nM in HBST (pH 7.4 or pH 5.5) was injected over the captured hPCSK9 surface at a flow rate of 100 μl/ml for 1.5 min at 25° C. and antigen-antibody dissociation was monitored for 10 min. Control I: anti-hPCSK9 mAb SEQ ID NO: 79/101 (WO 2008/063382) (K_{D}=pM; T_{1/2}=min).

TABLE 5

| | High hPCSK9 | | | | Low hPCSK9 | | | |
|---|---|---|---|---|---|---|---|---|
| | Density Surface | | | | Density Surface | | | |
| | pH 7.4 | | pH 5.5 | | pH 7.4 | | pH 5.5 | |
| Antibody | K_{D} | T_{1/2} | K_{D} | T_{1/2} | K_{D} | T_{1/2} | K_{D} | T_{1/2} |
| 316P | 191 | 74 | 144 | 83 | 339 | 45 | 188 | 58 |
| 300N | 65 | 507 | 1180 | 26 | 310 | 119 | 1380 | 13 |
| Control I | 20000 | 29 | ND | ND | ND | ND | ND | ND |

The antigen binding properties of 316P and 300N at pH 7.4 or pH 5.5 were determined by a modified BIACORE™ assay as described above. Briefly, mAbs were immobilized onto BIACORE™ CM5 sensor chips via amine coupling. Varying concentrations of myc-myc-his tagged hPCSK9, mouse PCSK9 (mPCSK9, SEQ ID NO: 757), hPCSK9 with a gain of function (GOF) point mutation of D374Y (hPCSK9 (D374Y), cynomolgus monkey (Macaca fascicularis) PCSK9 (mfPCSK9, SEQ ID NO: 761) (mfPCSK9), rat (Rattus norvegicus) PCSK9 (rPCSK9, SEQ ID NO:763), and his-tagged Syrian golden hamster (Mesocricetus auratus) PCSK9 (maPCSK9, SEQ ID NO: 762) (maPCSK9), ranging from 11 to 100 nM, were injected over the antibody surface at the flow rate of 100 μl/ml for 1.5 min and antigen-antibody dissociation was monitored in real time for 5 min at either 25° C. (Table 6) or 37° C. (Table 7). Control II: anti-hPCSK9 mAbs SEQ ID NO:67/12 (WO 2009/026558) (NB: no binding was observed under the experimental condition) (K_{D}=pM; T_{1/2}=min).

TABLE 6
pH Effect at 25° C.

| | pH 7.4 | | pH 5.5 | |
|---|---|---|---|---|
| Antigen | K_{D} | T_{1/2} | K_{D} | T_{1/2} |
| | 316P | | | |
| hPCSK9-mmh | 1260 | 36 | 22 | 39 |
| mPCSK9-mmh | 4460 | 10 | 63 | 11 |
| hPCSK9(D347Y)-mmh | 2490 | 15 | 166 | 13 |
| mfPCSK9-mmh | 1420 | 42 | 8 | 23 |
| maPCSK9-h | 8350 | 8 | 87 | 8 |
| rPCSK9-mmh | 24100 | 2 | 349 | 5 |
| | 300N | | | |
| hPCSK9-mmh | 1100 | 76 | 3100 | 5 |
| mPCSK9-mmh | NB | NB | NB | NB |
| hPCSK9(D347Y)-mmh | 1310 | 46 | 9030 | 3 |
| mfPCSK9-mmh | 2170 | 31 | 38500 | 0.4 |
| maPCSK9-h | NB | NB | NB | NB |
| rPCSK9-mmh | NB | NB | NB | NB |
| | Control I | | | |
| hPCSK9-mmh | 33100 | 14 | 1740 | 31 |
| mPCSK9-mmh | NB | NB | NB | NB |
| hPCSK9(D347Y)-mmh | 71000 | 11 | 7320 | 30 |
| mfPCSK9-mmh | 362000 | 0.2 | 67200 | 3 |
| maPCSK9-h | NB | NB | NB | NB |
| rPCSK9-mmh | NB | NB | NB | NB |
| | Control II | | | |
| hPCSK9-mmh | 143 | 266 | 2 | 212 |
| mPCSK9-mmh | 3500 | 11 | 33 | 12 |
| hPCSK9(D347Y)-mmh | 191 | 155 | 49 | 56 |
| mfPCSK9-mmh | 102 | 262 | 12 | 63 |
| maPCSK9-h | 6500 | 3 | ND | ND |
| rPCSK9-mmh | 22400 | 2 | 106 | 5 |

TABLE 7
pH Effect at 37° C.

| | pH 7.4 | | pH 5.5 | |
|---|---|---|---|---|
| Antigen | K_{D} | T_{1/2} | K_{D} | T_{1/2} |
| | 316P | | | |
| hPCSK9-mmh | 4000 | 9 | 142 | 11 |
| mPCSK9-mmh | 12200 | 3 | 13600 | 3 |
| hPCSK9(D347Y)-mmh | 6660 | 4 | 1560 | 5 |
| mfPCSK9-mmh | 3770 | 11 | 44 | 5 |
| maPCSK9-h | 21700 | 2 | ND | ND |
| rPCSK9-mmh | 55100 | 2 | 399 | 1 |
| | 300N | | | |
| hPCSK9-mmh | 2470 | 20 | 11900 | 1 |
| mPCSK9-mmh | NB | NB | NB | NB |
| hPCSK9(D347Y)-mmh | 2610 | 14 | 28000 | 1 |
| mfPCSK9-mmh | 4810 | 8 | 65200 | 0.1 |
| maPCSK9-h | NB | NB | NB | NB |
| rPCSK9-mmh | NB | NB | NB | NB |
| | Control I | | | |
| hPCSK9-mmh | 45900 | 0.1 | 11300 | 3 |
| mPCSK9-mmh | NB | NB | NB | NB |
| hPCSK9(D347Y)-mmh | 169000 | 0.4 | 27000 | 3 |
| mfPCSK9-mmh | 500000 | 0.6 | 5360 | 0.3 |
| maPCSK9-h | NB | NB | NB | NB |
| rPCSK9 | NB | NB | NB | NB |
| | Control II | | | |
| hPCSK9-mmh | 284 | 87 | 20 | 44 |
| mPCSK9-mmh | 8680 | 3 | 89 | 3 |
| hPCSK9(D347Y)-mmh | 251 | 57 | 483 | 26 |
| mfPCSK9-mmh | 180 | 127 | 214 | 65 |
| maPCSK9-h | 8830 | 0.5 | ND | ND |
| rPCSK9p-mmh | 30200 | 1 | 233 | 1 |

### Example 5. Anti-hPCSK9 mAbs Binding to hPCSK9 with Point Mutation D374Y

The binding affinity of selected anti-hPCSK9 mAbs to hPCSK9 with a gain of function (GOF) point mutation of D374Y (hPCSK9 (D374Y)-mmh) was determined as described above. Each antibody was captured at a flowrate of 40 μl/min for 8-30 sec on a goat anti-human IgG polyclonal antibody surface created through direct chemical coupling to a BIACORE™ chip to form a captured antibody surface. hPCSK9 (D374Y)-mmh at varying concentrations of 1.78 nM to 100 nM was injected over the captured antibody surface at a flowrate of 50 μl/min for 5 min, and the dissociation of hPCSK9 (D374Y)-mmh and antibody was monitored for 15 min at 25° C. Control III: anti-hPCSK9 mAbs SEQ ID NO: 49/23 (WO 2009/026558) (K_{D}=pM; T_{1/2}=min).

TABLE 8

| Antibody | K_{D} | T_{1/2} |
|---|---|---|
| 316P | 1780 | 14 |
| 300N | 1060 | 49 |
| Control I | 23600 | 25 |
| Control II | 66 | 216 |
| Control III | 1020 | 126 |

### Example 6. Binding Specificity of Anti-hPCSK9 mAbs

316P, 300N, and Control I anti-hPCSK9 mAbs were captured on an amine-coupled anti-hFc CM5 chip on BIACORE™ 2000. Tagged (myc-myc-his) human PCSK9, human PCSK1 (hPCSK1) (SEQ ID NO:759), human PCSK7 (hPCSK7) (SEQ ID NO:760), or mouse PCSK9 were injected (100 nM) over the captured mAb surface and allowed to bind at 25° C. for 5 min. Changes in RU were recorded. Results: 300N and Control I bound only to hPCSK9, and 316P bound both hPCSK9 and mPCSK9.

The binding specificities of anti-hPCSK9 mAbs were determined by ELISA. Briefly, anti-hPCSK9 antibody was coated on a 96-well plate. Human PCSK9-mmh, mPCSK9-mmh, maPCSK9-h, hPCSK1-mmh, or hPCSK7-mmh, at 1.2 nM, were added to antibody-coated plates and incubated at RT for 1 hr. Plate-bound PCSK protein was then detected by HRP-conjugated anti-His antibody. Results show that 316P binds human, mouse, and hamster PCSK9, whereas 300N and Control I only bound hPCSK9. None of the anti-hPCSK9 mAbs exhibited significant binding to hPCSK1 or hPCSK7.

### Example 7. Cross-Reactivity of Anti-hPCSK9 mAbs

Cross-reactivity of anti-hPCSK9 mAbs with mmPCSK9, mfPCSK9, mPCSK9, maPCSK9, or rPCSK9 was determined using BIACORE™ 3000. Briefly, anti-hPCSK9 mAbs were captured on an anti-hFc surface created through direct chemical coupling to a BIACORE™ chip. Purified tagged hPCSK9, hPCSK9 (D374Y), mmPCSK9, mfPCSK9, mPCSK9, maPCSK9, or rPCSK9, each at 1.56 nM to 50 nM, was injected over the antibody surface at either 25° C. or 37° C. Binding between 316P, 300N, Control I, Control II, or Control III and the PCSK9 proteins was determined (K_{D}=pM; T_{1/2}=min) (ND=not determined).

TABLE 9
316P mAb

| | 37° C. | | 25° C. | |
|---|---|---|---|---|
| Antigen | K_{D} | T_{1/2} | K_{D} | T_{1/2} |
| hPCSK9-mmh | 1800 | 9 | 580 | 36 |
| hPCSK9(D374Y)-mmh | 4200 | 4 | 1690 | 15 |
| mmPCSK9-mmh | 1800 | 21 | 550 | 92 |
| mfPCSK9-mmh | 1800 | 11 | 520 | 60 |
| mPCSK9-mmh | 4700 | 3 | 2300 | 11 |
| maPCSK9-h | 19000 | 1 | 6810 | 5 |
| rPCSK9-mmh | 37500 | 1 | 14500 | 2 |

TABLE 10
300N mAb

| | 37° C. | | 25° C. | |
|---|---|---|---|---|
| Antigen | K_{D} | T_{1/2} | K_{D} | T_{1/2} |
| hPCSK9-mmh | 2400 | 22 | 740 | 110 |
| hPCSK9(D374Y)-mmh | 2200 | 14 | 900 | 65 |
| mmPCSK9-mmh | 1600 | 26 | 610 | 79 |
| mfPCSK9-mmh | 3800 | 11 | 1500 | 45 |
| mPCSK9-mmh | NB | NB | NB | NB |
| maPCSK9-h | NB | NB | NB | NB |
| rPCSK9-mmh | NB | NB | NB | NB |

TABLE 11
Control I mAb

| | 37° C. | | 25° C. | |
|---|---|---|---|---|
| Antigen | K_{D} | T_{1/2} | K_{D} | T_{1/2} |
| hPCSK9-mmh | 226000 | 2 | 27500 | 16 |
| hPCSK9(D374Y)-mmh | ND | ND | 23600 | 25 |
| mmPCSK9-mmh | 420000 | 3 | 291000 | 2 |
| mfPCSK9-mmh | 14300 | 10 | 24900 | 14 |
| mPCSK9-mmh | NB | NB | NB | NB |
| maPCSK9-h | NB | NB | NB | NB |
| rPCSK9-mmh | NB | NB | NB | NB |

TABLE 12
Control II mAb

| | 37° C. | | 25° C. | |
|---|---|---|---|---|
| Antigen | K_{D} | T_{1/2} | K_{D} | T_{1/2} |
| hPCSK9-mmh | 91 | 162 | 61 | 372 |
| hPCSK9(D374Y)-mmh | 93 | 90 | 66 | 216 |
| mfPCSK9-mmh | 33 | 252 | 26 | 546 |
| mPCSK9-mmh | 4700 | 3 | 2300 | 11 |
| maPCSK9-h | 60800 | 0.4 | 25000 | 2 |
| rPCSK9-mmh | 14100 | 1 | 6900 | 3 |

TABLE 13
Control III mAb

| | 37° C. | | 25° C. | |
|---|---|---|---|---|
| Antigen | K_{D} | T_{1/2} | K_{D} | T_{1/2} |
| hPCSK9-mmh | 380 | 378 | 490 | 450 |
| hPCSK9(D374Y)-mmh | 130 | 660 | 1000 | 126 |
| mfPCSK9-mmh | 110 | 750 | 340 | 396 |
| mPCSK9-mmh | 33500 | 1 | 10900 | 4 |
| maPCSK9-h | 780 | 107 | 2100 | 67 |
| rPCSK9-mmh | NB | NB | 33200 | 2 |

### Example 8. Inhibition of Binding Between hPCSK9 and hLDLR Domains

The ability of selected anti-hPCSK9 mAbs to block hPCSK9 binding to human LDLR full-length extracellular domain (hLDLR-ecto SEQ ID NO:758), hLDLR EGF-A domain (amino acids 313-355 of SED ID NO: 758), or hLDLR EGF-AB domains (amino acids of 314-393 of SEQ ID NO:758) (LDLR Genbank number NM_000527) was evaluated using BIACORE™ 3000. Briefly, hLDLR-ecto, EGF-A-hFc, or EGF-AB-hFc protein was amine-coupled on a CM5 chip to create a receptor or receptor fragment surface. Selected anti-hPCSK9 mAbs, at 62.5 nM (2.5 fold excess over antigen), were premixed with 25 nM of hPCSK9-mmh, followed by 40 min incubation at 25° C. to allow antibody-antigen binding to reach equilibrium to form equilibrated solutions. The equilibrated solutions were injected over the receptor or receptor fragment surfaces at 2 μl/min for 40 min at 25° C. Changes in RU due to the binding of the anti-hPCSK9 mAbs to hLDLR-ecto, EGF-A-hFc, or EGF-AB-hFc were determined. Results show that H1H316P and H1M300N blocked the binding of hPCSK9-mmh to hLDLR-ecto, hLDLR EGF-A domain, and hLDLR EGF-AB domains; H1H320P blocked the binding of hPCSK9-mmh to hLDLR-ecto and hLDLR EGF-A domain; and H1H321P blocked the binding of hPCSK9-mmh to hLDLR EGF-A domain.

The ability of the mAbs to block hPCSK9 binding to hLDLR-ecto, hLDLR EGF-A domain, or hLDLR EGF-AB domains was also evaluated with an ELISA-based immunoassay. Briefly, hLDLR-ecto, hLDLR EGF-A-hFc or hLDLR EGF-AB-hFc, each at 2 μg/ml, was coated on a 96-well plate in PBS buffer overnight at 4° C., and nonspecific binding sites blocked with BSA. This plate was used to measure free hPCSK9-mmh in a PCSK9-mmh solution pre-equilibrated with varying concentrations of anti-hPCSK9 mAbs. A constant amount of hPCSK9-mmh (500 pM) was pre-mixed with varied amounts of antibody, ranging from 0 to ˜50 nM in serial dilutions, followed by 1 hr incubation at room temperature (RT) to allow antibody-antigen binding to reach equilibrium. The equilibrated sample solutions were transferred to receptor or receptor fragment coated plates. After 1 hour of binding, the plates were washed and bound hPCSK9-mmh detected using HRP conjugated anti-myc antibody. IC₅₀ values (in pM) were determined as the amount of antibody required to achieve 50% reduction of hPCSK9-mmh bound to the plate-coated receptor or receptor fragment. The results show that specific mAbs functionally block PCSK9 from binding the three receptors at both neutral pH (7.2) and acidic pH (5.5).

TABLE 14

| | pH 7.2 | | | pH 5.5 | | |
|---|---|---|---|---|---|---|
| | Plate Coating Surface | | | | | |
| Ab | hLDLR-ecto | EGF-A | EGF-AB | hLDLR-ecto | EGF-A | EGF-AB |
| 316P | <125 | <125 | <125 | <125 | <125 | <125 |
| 300N | 144 | 146 | <125 | 1492 | 538 | 447 |
| Control I | — | >100,000 | >100,000 | — | >100,000 | >100,000 |
| Control II | 288 | 510 | 274 | 411 | 528 | 508 |
| Control III | 303 | 635 | 391 | 742 | 787 | 1073 |

The ability of the mAbs to block hPCSK9 GOF mutant hPCSK9 (D374Y)-mmh binding to hLDLR EGF-A domain or hLDLR EGF-AB domain (IC₅₀ values in pM) was also evaluated with the ELISA-based immunoassay described above using a constant amount of 0.05 nM hPCSK9 (D374Y)-mmh.

TABLE 15

| | pH 7.2 | | pH 5.5 | |
|---|---|---|---|---|
| | Plate Coating Surface | | | |
| | EGF-A | EGF-AB | EGF-A | EGF-AB |
| 316P | 203 | 139 | 1123 | 1139 |
| 300N | 135 | 142 | 3463 | 3935 |
| Control I | >100,000 | >100,000 | >100,000 | >100,000 |
| Control II | 72 | 57 | 129 | 118 |
| Control III | 537 | 427 | 803 | 692 |

The ability of the mAbs to block either mmPCSK9 or mPCSK9 binding to hLDLR-ecto domain, hLDLR EGF-A domain, or hLDLR EGF-AB domain (IC₅₀ values in pM) was evaluated at neutral pH (7.2) with the ELISA-based immunoassay describe above using a constant amount of 1 nM of mmh-tagged mmPCSK9 or 1 nM of mPCSK9.

TABLE 16

| | 1 nM mmPCSK9-mmh | | | 1 nM mPCSK9-mmh | |
|---|---|---|---|---|---|
| | hLDLR- | EGF- | EGF- | EGF- | EGF- |
| | ecto | A | AB | A | AB |
| 316P | <250 | <250 | <250 | <250 | <250 |
| 300N | 255 | 256 | 290 | >33000 | >33000 |

The ability of the mAbs to block hPCSK9, mmPCSK9, rPCSK9, maPCSK9, mfPCSK9, or mPCSK9 binding to hLDLR EGF-A domain (IC₅₀ values in pM) was evaluated at neutral pH (7.2) (Table 17) acidic pH (5.5, Table 18) with the ELISA-based immunoassay described above using a constant amount of 0.5 nM of hPCSK9-mmh, 1 nM of mmPCSK9-mmh, 1 nM of rPCSK9-mmh, 1 nM of maPCSK9-h, 0.3 nM of mfPCSK9-mmh, or 1 nM of mPCSK9-mmh.

TABLE 17

| | hPCSK9 | mmPCSK9 | rPCSK9 | maPCSK9 | mfPCSK9 | mPCSK9 |
|---|---|---|---|---|---|---|
| 316P | <125 | <250 | 2662 | 349 | 75 | 305 |
| 300N | 182 | 460 | >100000 | >100000 | 473 | >100000 |
| Control I | — | >100000 | >100000 | >100000 | >100000 | >100000 |
| Control II | 146 | 83 | 2572 | 2038 | 361 | 855 |
| Control III | 249 | 293 | >100000 | 245 | 572 | >100000 |

TABLE 18

| | hPCSK9 | mmPCSK9 | rPCSK9 | maPCSK9 | mPCSK9 |
|---|---|---|---|---|---|
| 316P | <125 | <250 | 42880 | 1299 | 991 |
| 300N | 223 | 3704 | >100000 | >100000 | >100000 |
| Control I | >10000 | >100000 | >100000 | >100000 | >100000 |
| Control II | 154 | <250 | 11640 | 8339 | 2826 |
| Control | 390 | 376 | >100000 | 414 | >100000 |
| III | | | | | |

The ability of 316P and Control I to block hPCSK9 binding to hLDLR was also determined. Briefly, either recombinant hLDLR or hLDLR-EGFA-mFc was immobilized onto BIACORE™ CM5 chips via amine coupling. An antigen-antibody mixture of 100 nM hPCSK9-mmh and 316P, Control I mAb, or a non-hPCSK9 specific mAb (each at 250 nM) was incubated at RT for 1 hr, and then injected over the hLDLR or hLDLR-EGFA surface at the flow rate of 10 μl/ml for 15 min at 25° C. Changes in RU due to the binding between the free hPCSK9-mmh in the mixture to either hLDLR or hLDLR-EGFA were recorded. The binding of hPCSK9 to either hLDLR or hLDLR-EGFA was completely blocked by 316P and 300N but not by Control I mAb.

### Example 9. Epitope Mapping

In order to determine epitope-binding specificity, three chimeric PCSK9-mmh proteins were generated in which specific human PCSK9 domains were substituted with mouse PCSK9 domains. Chimeric protein #1 consists of a mouse PCSK9 pro-domain (amino acid residues 1-155 of SEQ ID NO:757) followed by a human PCSK9 catalytic domain (residues 153-425 of SEQ ID NO:755) and a mouse PCSK9 C-terminal domain (residues 429-694 SEQ ID NO:757) (mPro-hCat-mC-term-mmh). Chimeric protein #2 consists of a human PCSK9 pro-domain (residues 1-152 of SEQ ID NO:755) followed by a mouse PCSK9 catalytic domain (residues 156-428 of SEQ ID NO:757) and a mouse PCSK9 C-terminal (hPro-mCat-mC-term-mmh). Chimeric protein #3 consists of mouse PCSK9 pro-domain and a mouse PCSK9 catalytic domain followed by a human PCSK9 C-terminal domain (residues 426-692 of SEQ ID NO:755) (mPro-mCat-hC-term-mmh). In addition, hPCSK9 with a point mutation of D374Y (hPCSK9 (D374Y)-mmh) was generated.

Binding specificity of mAbs to test proteins hPCSK9-mmh, mouse PCSK9-mmh, chimeric proteins #1, #2, and #3, and hPCSK9 (D374Y)-mmh were tested as follows: the mAbs were coated on a 96-well plate overnight at 4° C., then each test protein (1.2 nM) was added to the plate. After 1 hr binding at RT, the plate was washed and bound test protein detected using HRP-conjugated anti-myc polyclonal antibody (++=OD>1.0; +=OD 0.4-1.0; −=OD<0.4).

TABLE 19

| | | | Chimeric Protein | | | hPCSK9 |
|---|---|---|---|---|---|---|
| Antibody | hPCSK9 | mPCSK9 | #1 | #2 | #3 | (D374Y) |
| H1M300 | ++ | − | ++ | + | − | ++ |
| H1M309 | ++ | − | − | − | ++ | ++ |
| H1M312 | ++ | − | − | − | ++ | ++ |
| H1M492 | ++ | − | − | − | − | + |
| H1M493 | ++ | − | − | − | ++ | ++ |
| H1M494 | ++ | − | − | + | ++ | ++ |
| H1M495 | ++ | − | − | − | ++ | ++ |
| H1M496 | ++ | − | − | − | ++ | ++ |
| H1M497 | ++ | − | − | ++ | + | ++ |
| H1M498 | ++ | − | − | − | + | ++ |
| H1M499 | ++ | − | − | − | ++ | ++ |
| H1M500 | ++ | − | ++ | − | − | ++ |
| H1M502 | ++ | − | − | − | ++ | ++ |
| H1M503 | ++ | − | − | ++ | − | ++ |
| H1M504 | ++ | − | − | − | − | + |
| H1M505 | ++ | − | ++ | + | − | ++ |
| H1M508 | ++ | − | − | − | ++ | ++ |
| H1H318P | ++ | − | ++ | − | − | ++ |
| H1H334P | ++ | − | ++ | − | − | ++ |
| H1H316P | ++ | ++ | ++ | ++ | ++ | ++ |
| H1H320P | ++ | − | − | ++ | − | ++ |
| Control I | ++ | − | − | − | ++ | ++ |

Binding specificity of 316P, 300N and control anti-hPCSK9 mAbs to hPCSK9-mmh, mPCSK9-mmh, mmPCSK9-mmh, mfPCSK9-mmh, rPCSK9-mmh, chimeric proteins #1, #2, and #3, and hPCSK9 (D374Y)-mmh were tested as described above except that the protein concentration is 1.7 nM (−=OD<0.7; +=OD 0.7-1.5; ++=OD>1.5).

TABLE 20

| | | | Control | Control | Control |
|---|---|---|---|---|---|
| | 316P | 300N | I | II | III |
| hPCSK9-mmh | ++ | ++ | ++ | ++ | ++ |
| mPCSK9-mmh | ++ | − | − | ++ | ++ |
| mmPCSK9-mmh | ++ | ++ | ++ | ++ | ++ |
| mfPCSK9-mmh | ++ | ++ | ++ | ++ | ++ |
| rPCSK9-mmh | ++ | − | − | ++ | + |
| Chimeric Protein #1 | ++ | ++ | − | ++ | ++ |
| Chimeric Protein #2 | ++ | ++ | − | ++ | ++ |
| Chimeric Protein #3 | ++ | + | ++ | ++ | ++ |
| hPCSK9 (D374Y) | ++ | ++ | ++ | ++ | ++ |

Similar results for selected mAbs were obtained by BIACORE™ binding assay. Briefly, 316P, 300N, or Control I mAb was captured on an amine-coupled anti-hFc CM5 chip and 100 nM of each protein injected over the mAb-captured surface. Changes in RU due to the binding of each protein to the mAb surface was determined.

TABLE 21

| | | | Chimeric Protein | | |
|---|---|---|---|---|---|
| Antibody | hPCSK9 | mPCSK9 | #1 | #2 | #3 |
| 316P | 500 | 505 | 529 | 451 | 467 |
| 300N | 320 | 13 | 243 | 76 | 10 |
| Control I | 65 | 7 | 4 | 3 | 69 |

To further assess the binding specificity of 316P, which cross-reacts with mPCSK9-mmh, a cross-competition ELISA assay was developed to determine binding domain specificity. Briefly, mAbs specific for chimeric protein #1, #2, or #3, were first coated on a 96-well plate overnight at 1 μg/ml. Human PCSK9-mmh (2 μg/ml) was then added to each well followed by 1 hr incubation at RT. 316P (1 μg/ml) was added and incubated for another hour at RT. Plate-bound 316P was detected using HRP-conjugated anti-hFc polyclonal antibody. Although 316P binding to hPCSK9-mmh was not affected by the presence of mAbs specific for either chimeric protein #2 or chimeric protein #3, 316P binding to hPCSK9-mmh was greatly reduced by the presence of antibody specific for chimeric protein #1.

### Example 10. BIACORE™-Based Antigen Binding Profile Assessment

Antibody binding profiles were also established for 316P, 300N, Control I, II, and III mAbs using BIACORE™ 1000. Briefly, hPCSK9-mmh was captured on an anti-myc surface. A first anti-hPCSK9 mAb (50 μg/ml) was injected over the PCSK9-bound surface for 10 min, at a flow rate of 10 μl/min at 25° C. A second anti-hPCSK9 mAb (50 μg/ml) was then injected over the first mAb-bound surface for 10 min, at a flow rate of 10 μl/min at 25° C. Ability of the first mAb to block binding of the second mAb was measured and is expressed as percent inhibition.

TABLE 22

| | Second mAb | | | | |
|---|---|---|---|---|---|
| First mAb | 316P | 300N | Control I | Control II | Control III |
| 316P | 100 | 101 | 27 | 99 | 101 |
| 300N | 77 | 100 | 12 | 82 | −2 |
| Control I | 6 | 12 | 100 | 6 | 9 |
| Control II | 91 | 102 | −6 | 100 | 3 |
| Control III | 73 | 10 | −12 | 1 | 100 |

### Example 11. Increase of LDL Uptake by Anti-hPCSK9 Antibodies

The ability of anti-hPCSK9 mAbs to increase LDL uptake in vitro was determined using a human hepatocellular liver carcinoma cell line (HepG2). HepG2 cells were seeded onto 96-well plates at 9×10⁴ cells/well in DMEM complete media and incubated at 37° C., 5% CO₂, for 6 hr to form HepG2 monolayers. Human PCSK9-mmh, at 50 nM in lipoprotein deficient medium (LPDS), and a test mAb was added in various concentrations from 500 nM to 0.98 nM in LPDS medium. Data are expressed as IC₅₀ values for each experiment (IC₅₀=antibody concentration at which increases LDL uptake by 50%). In addition, the experiment also showed that both 316P and 300N were able to completely reverse the inhibitory effect of hPCSK9 on LDL uptake, while Control I mAb or H1M508 anti-hPCSK9 mAb reversed the inhibitory effect by about 50%.

TABLE 23

| Antibody | IC₅₀ (nM) |
|---|---|
| 316P | 21.30 |
| 300N | 22.12 |
| Control I | >250 |
| H1M508 | >250 |

The ability of anti-hPCSK9 mAbs to reverse the inhibitory effect on LDL uptake by PCSK9 protein from different mammalian species was also tested in a HepG2 cell line as described above. Briefly, HepG2 cells were incubated overnight with serial dilutions of antibody in LPDS medium (beginning with 500 nM) and 50 nM of hPCSK9-mmh, mfPCSK9-mmh, mPCSK9-mmh, rPCSK9-mmh, or maPCSK9-h. HepG2 cells were also incubated overnight with serial dilutions of antibody in LPDS (beginning with 50 nM) and 1 nM hPCSK9 (D374Y). As shown in Table 24, while 316P was able to completely reverse the inhibitory effect on LDL by all PCSK9 proteins tested, 300N was only able to reverse the inhibitory effect on LDL uptake by hPCSK9, hPCSK9 (D374Y), and mfPCSK9. Values are expressed as nM IC₅₀.

TABLE 24

| | 316P | 300N | Control I | Control II | Control III |
|---|---|---|---|---|---|
| hPCSK9-mmh | 14.1 | 12.6 | >500 | 13.4 | 12.4 |
| hPCSK9(D374Y)- | 2.1 | 1.1 | >50 | 0.7 | 0.6 |
| mmh | | | | | |
| mfPCSK9-mmh | 14.7 | 13.4 | >500 | 14.2 | 13.6 |
| mPCSK9-mmh | 21.2 | >500 | >500 | 19 | >500 |
| rPCSK9-mmh | 27.7 | >500 | >500 | 21.9 | >500 |
| maPCSK9-h | 14.4 | >500 | >500 | 29.5 | 12.7 |

### Example 12. Neutralization of Biological Effect of hPCSK9 In Vivo

To assess the biological effect of neutralizing PCSK9, hPCSK9 was over-expressed in C57BL/6 mice by hydrodynamic delivery (HDD) of DNA constructs encoding full-length hPCSK9-mmh. 4 mice (C57BL/6) were injected with empty vector/saline (control), and 16 mice were injected with a 50 μg hPCSK9-mmh-DNA/saline mixture in the tail vein equal to 10% of their body weight. At day 7 after HDD, delivery of hPCSK9 resulted in a 1.6-fold elevation of total cholesterol, 3.4-fold elevation in LDL-cholesterol (LDL-C) and a 1.9-fold elevation in non-HDL cholesterol (relative to control). Serum hPCSK9 levels on day 7 were all greater than 1 μg/ml, as assessed by quantitative ELISA.

Administration of H1M300N on day 6 after HDD to 3 experimental groups (1, 5 or 10 mg/kg) (n=4 per group) via intraperitoneal (i.p.) injection resulted in a significant attenuation of serum cholesterol levels. At 18 hours after administration, total cholesterol was reduced by 9.8%, 26.3% and 26.8%, LDL-C was reduced by 5.1%, 52.3% and 56.7%, and non-HDL cholesterol was reduced by 7.4%, 33.8% and 28.6% in the 1, 5 or 10 mg/kg H1M300N treated groups, respectively.

### Example 13. Pharmacokinetic and Serum Chemistry Study in Monkeys

A pharmacokinetic (PK) study was conducted in naïve male cynomolgus monkeys (Macaca fascicularis) with a body weight range between 5-7 kg and aged between 3-5 years.

Group assignments. The monkeys were assigned into 5 treatment groups: Treatment Group 1 (n=3) received control buffer (10 mM sodium phosphate, pH 6, 1 ml/kg); Treatment Group 2 (n=3) received 1 ml/kg of 316P (5 mg/ml); Treatment Group 3 (n=3) received 1 ml/kg 300N (5 mg/ml); Treatment Group 4 (n=3) received 1 ml/kg 316P (15 mg/ml); and Treatment Group 5 (n=3) received 1 ml/kg 300N (15 mg/ml). All treatments were administered by IV bolus followed by a 1 ml saline flush. Total dose volume (ml) was calculated on the most recent body weight (each animal was weighed twice during acclimation and once weekly throughout the study). A single dose of test mAb or buffer control was administered on Day 1.

Animal care. Animals were housed in a temperature-and humidity-monitored environment. The targeted range of temperature and relative humidity was between 18-29° C. and 30-70%, respectively. An automatic lighting system provided a 12-hour diurnal cycle. The dark cycle could be interrupted for study-or facility-related activities. The animals were individually housed in cages that comply with the Animal Welfare Act and recommendations set forth in The Guide for the Care and Use of Laboratory Animals (National Research Council 1996).

Diet and Feeding. Animals were fed twice per day according to SNBL USA SOPs. Animals were fasted when required by specific procedures (e.g., prior to blood draws for serum chemistry, urine collection, or when procedures involving sedation are performed). The diet was routinely analyzed for contaminants and found to be within manufacturer's specifications. No contaminants were expected to be present at levels that would interfere with the outcome of the study.

Experimental Design. An appropriate number of animals were selected from SNBL USA stock. Animals were examined for health by veterinary staff, and had undergone serum chemistry, hematology, and coagulation screening. Sixteen males, confirmed healthy, were assigned to the study. Fifteen males were assigned to specific study groups and the remaining animal was available as a spare. A stratified randomization scheme incorporating serum cholesterol level (based on the average of two draws in acclimation) was used to assign animals to study groups.

Acclimation Period. Previously quarantined animals were acclimated to the study room for a minimum of 14 days prior to initiation of dosing. Acclimation phase data was collected from all animals, including the spare. All animals were assessed for behavioral abnormalities that could affect performance on study. The spare animal was returned to stock after day 1.

Blood collection. Blood was collected by venipuncture from a peripheral vein from restrained, conscious animals. Whenever possible, blood was collected via a single draw and then divided appropriately.

PK Study. Blood samples (1.5 ml) were collected at pre-dose, 2 min, 15, min, 30 min, 1 hr, 2 hr, 4 hr, 8 hr, 12 hr, 24 hr, and subsequently once every 24 hr in serum separator tubes (SST). Specimen storage serum is transferred to 2 vials and stored at −60° C. or below.

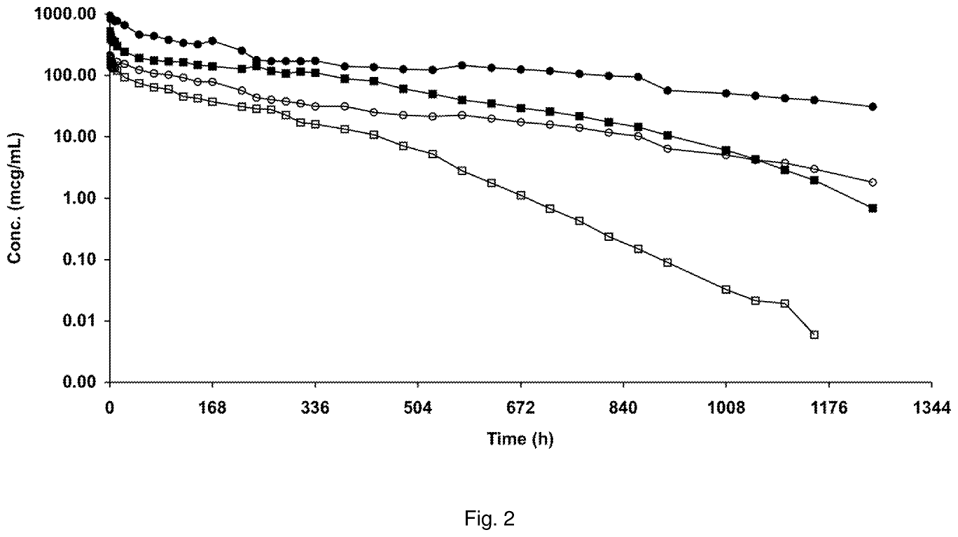

Serum samples were analyzed using an optimized ELISA (enzyme-linked immunosorbant assay) procedure. Briefly, a microtiter plate was first coated with hPCSK9-mmh. Test mAb 316P or 300N was then captured on the hPCSK9-mmh plate. The captured 316P or 300N was detected using a biotinylated mouse anti-hIgG4 followed by binding to NeutrAvidin-HRP. Varying concentrations of 316P or 300N, ranging from 100 to 1.56 ng/ml, were used as standards. One percent monkey serum (assay matrix) in the absence of 316P or 300N was used as the zero (0 ng/ml) standard. The results, shown in FIG. 2, indicate a dose-dependent increase in serum 316P and 300N levels. PK parameters were analyzed using WinNonlin software (Noncompartmental analysis, Model 201-IV bolus administration).

TABLE 25

| | 316P | | 300N | |
|---|---|---|---|---|
| PK Parameter | 5 mg/kg | 15 mg/kg | 5 mg/kg | 15 mg/kg |
| Tₘₐₓ (h) | 0.428 | 0.105 | 4.02 | 0.428 |
| Cₘₐₓ (μg/ml) | 184 | 527 | 226 | 1223 |
| T_{1/2} (h) | 83 | 184 | 215 | 366 |

Serum Chemistry. Blood samples were collected at pre-dose, 12 hr, 48 hr, and subsequently once every 48 hr, for clinical chemistry analysis, in particular lipid profiles (i.e. cholesterol, LDL-C, HDL-C, triglycerides). With the exception of the 12 hr post-dose sample, all animals were subject to an overnight fast prior to sample collection. The sample volume was approximately 1 ml. Chemistry parameters were determined using an Olympus automated analyzer. Parameters measured (Xybion code): Albumin (ALB); Alkaline Phosphatase (ALP); Alanine Aminotransferase (ALT); Aspartate Transaminase (AST); Total Bilirubin (TBIL); Calcium (Ca); Total Cholesterol (TCho); Creatine Kinase (CK); Creatinine (CRN); Gamma Glutamyltransaminase (GGT); Glucose (GLU); Inorganic Phosphorus (IP); Total Protein (TP); Triglyceride (TRIG); Blood Urea Nitrogen (BUN); Globulin (GLOB); Albumin/Globulin Ratio (A/G); Chloride (CI); Potassium (K); Sodium (Na); LDL and HDL cholesterol. Residual serum was stored at −20° C. or below and disposed of no sooner than one week after analysis.

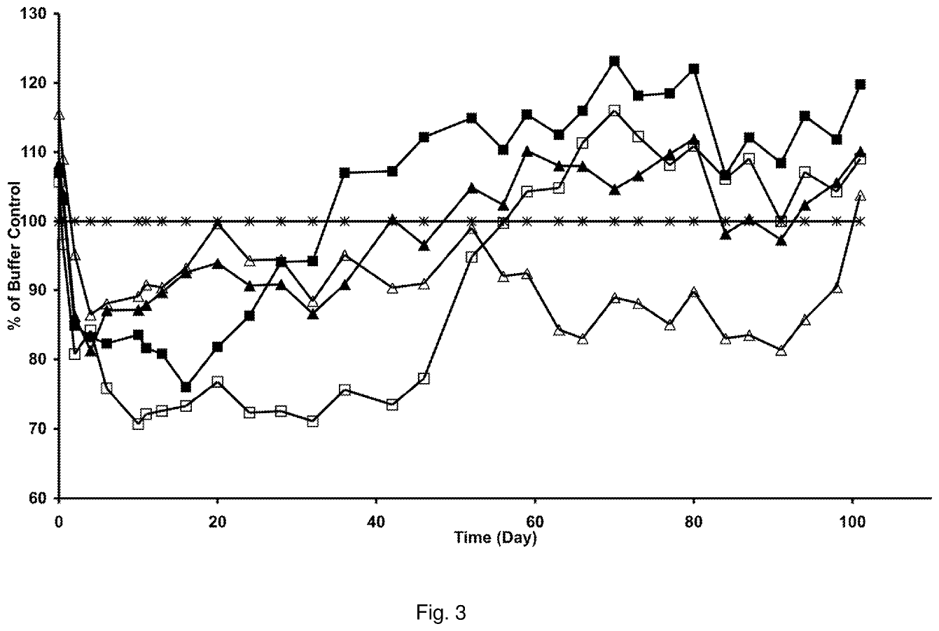

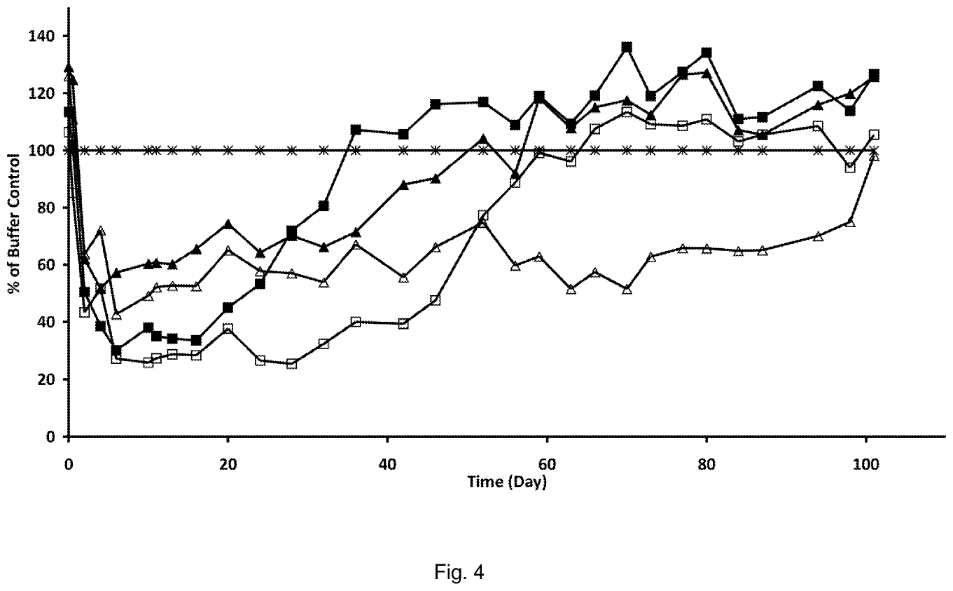

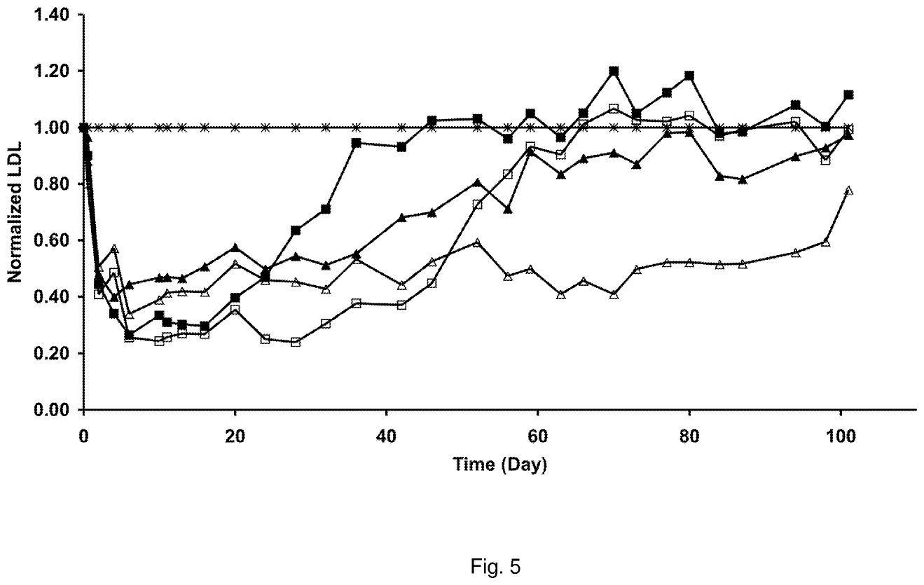

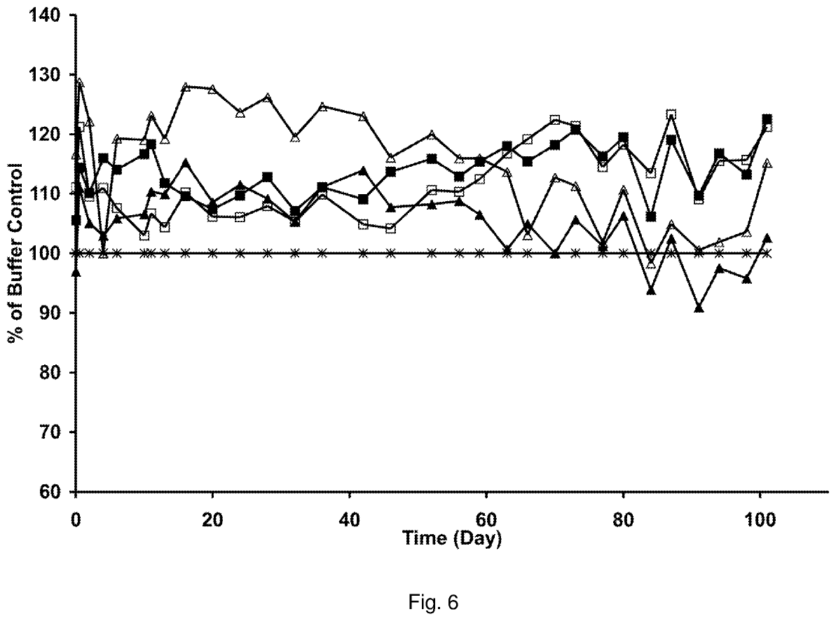

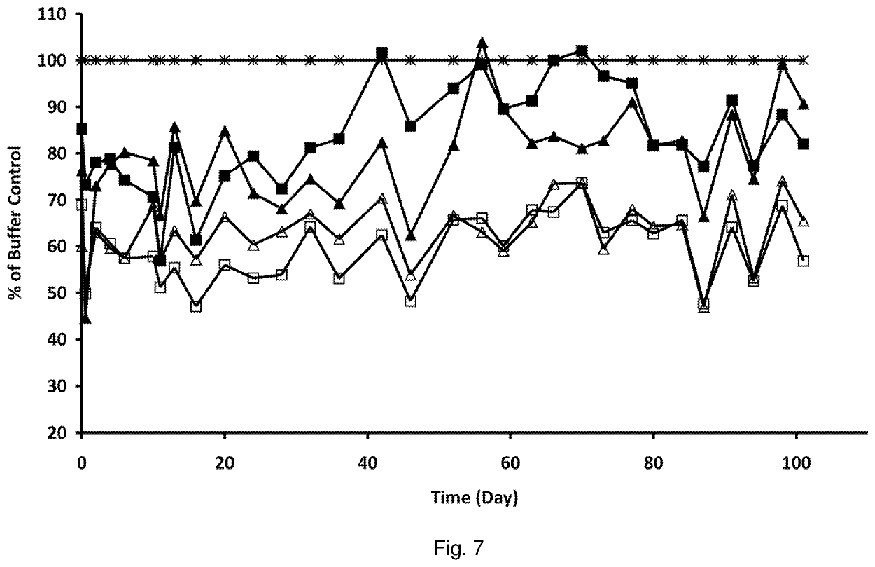

Results from samples through Day 105 post-dose time point are shown in FIGS. 3-7. There was a reduction in total cholesterol and LDL-C in animals receiving 316P and 300N, regardless of dose, within 24 hours of the first dose. Serum total cholesterol reduced rapidly and robustly (˜35%, FIG. 3). A robust decrease of ˜80% was seen in LDL-C (FIGS. 4-5) by day 6. In animals that received a 15 mg/kg dose of 300N, the reduction in both total cholesterol (˜10-15% reduction) and LDL-C (˜40% reduction) continued to at least day 80 of the study. In addition, HDL-C was elevated in animals that received 316P at 15 mg/kg (FIG. 6). Animals that received a higher dose (15 mg/kg) of either 316P or 300N also showed a reduction in triglycerides during the course of study (FIG. 7). 316P exhibited maximal suppression of LDL-C levels of up to 80% relative to baseline. The length of this suppression was dose-dependent with at least 60% suppression (relative to baseline LDL-C levels) lasting approximately 18 days (5 mg/kg dose) and approximately 45 days (15 mg/kg dose). 300N exhibits a distinct pharmacodynamic profile from 316P. LDL-C suppression by 300N was sustained for a much longer period of time at comparable doses (50% LDL-C suppression for 28 days following a 5 mg/kg dose and 50% LDL-C suppression for approximately 90 days following a 15 mg/kg dose). There was little or no measurable change in liver function as determined by ALT and AST measurements. All animals receiving an anti-PCSK9 antibody in the study exhibited a rapid suppression If LDL-C and total cholesterol.

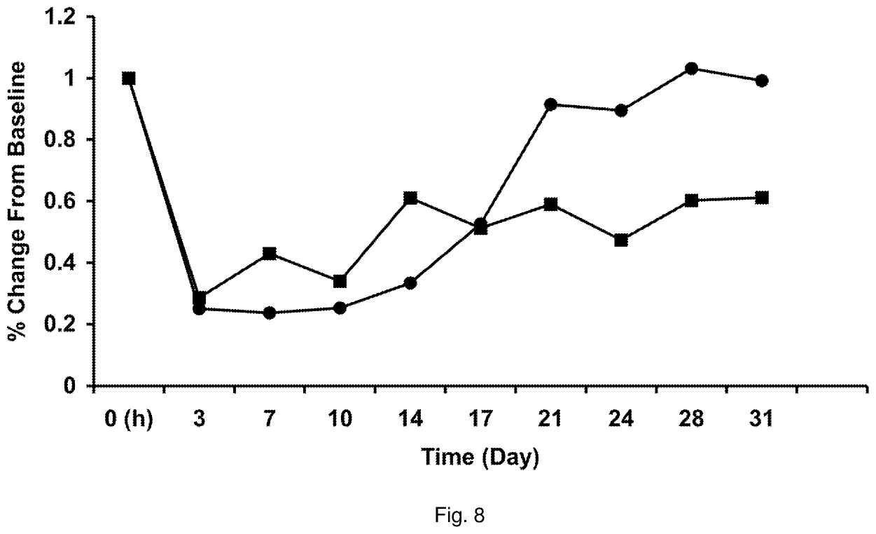

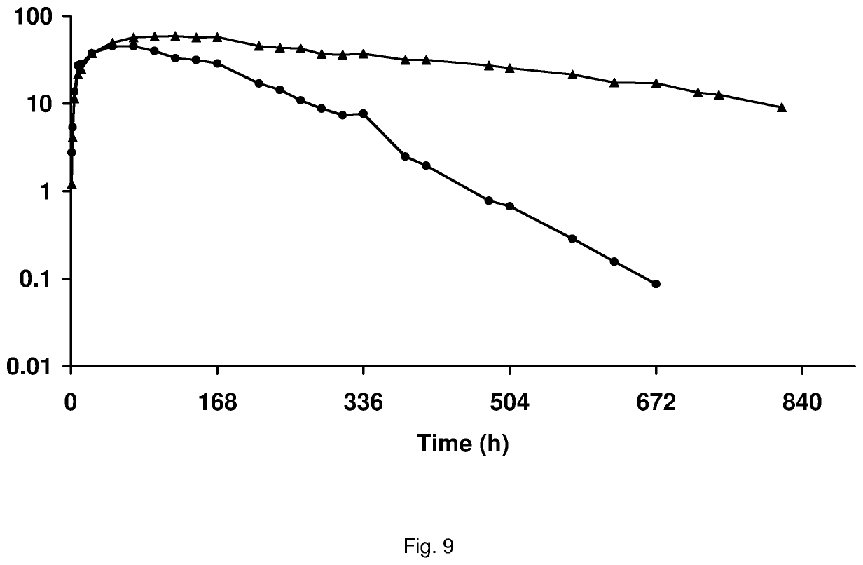

A similar LDL-C lowering effect of 316P and 300N was also observed in cynomolgous monkeys that received a single subcutaneous (SC) administration of either 5 mg/kg 316P or 5 mg/kg 300N (FIG. 8). Both 316P and 300N dramatically suppressed LDL-C levels and maintained an LDL-C lowering effect for approximately 15 and 30 days, respectively (FIG. 8). The pharmacodynamic effect (approximately 40% LDL-C suppression) approximately correlates with functional antibody levels in monkey serum (FIG. 9). As antibody levels decrease below 10 μg/ml, LDL-C suppression appeared to diminish as well. In addition, 300N demonstrated a substantially longer circulating half-life than 316P and hence a longer observed LDL-C suppression.

TABLE 26

| PK Parameter | 316P | 300N |
|---|---|---|
| Tₘₐₓ (h) | 60 | 84 |
| Cₘₐₓ (μg/ml) | 46 | 63 |
| T_{1/2} (h) | 64 | 286 |

### Example 14. Attenuation of LDL Receptor Degradation by Anti-hPCSK9 Antibodies

To assess the biological effect of PCSK9 on hepatic LDL receptor levels and subsequent effects on serum LDL-C levels, hPCSK9 was administered to mice expressing hPCSK9 but not mPCSK9 (PCSK9^{hu/hu} mice) by intravenous injection. Specifically, PCSK9^{hu/hu} mice were injected with PBS (control), or 1.2 mg/kg hPCSK9-mmh via the tail vein. Six hours after delivery of hPCSK9, a 1.4-fold elevation (relative to baseline level) in total cholesterol and a 2.3-fold elevation in LDL-C) in serum were observed. Analysis of hepatic LDL receptor levels in a separate cohort (n=3) of animals 4 hours after hPCSK9 administration revealed a significant reduction in detectable LDL receptor in liver homogenates.

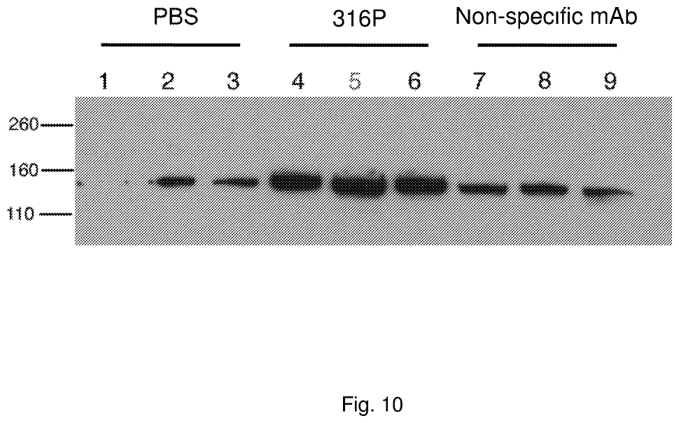

To assess the biological effect of anti-hPCSK9 on hepatic LDL receptor levels and subsequent effects on serum LDL-C levels, 316P and a non-hPCSK9 specific mAb were administered to PCSK9^{hu/hu} mice at equivalent dose (5 mg/kg i.p.) 20 hours prior to the hPCSK9-mmh protein injection described above. Four hours after the hPCSK9 administration, mice were sacrificed, a total of eight tissues (liver, brain, lung, kidney, heart, ileum, adrenal, and pancreas) were collected, and levels of LDL receptor were determined by Western blot. Changes in LDL receptor levels were only observed in liver. In comparison to PBS control dosing, administration of 316P significantly blocked the PCSK9-mediated increases in total cholesterol and LDL cholesterol (LDL-C=2.49 mg/dl at baseline and 3.1 mg/dl 6 hours after PCSK9; a 25% increase compared to 135% with vehicle). Prior administration of the non-hPCSK9 specific mAb blocked LDL-C increases by approximately 27% from PBS alone (LDL-C=4.1 mg/dl compared to PBS 5.6 mg/dl). Analysis of LDL receptor levels in a separate cohort of mice (n=3 per treatment group) revealed a significant reduction in LDL receptor levels with PCSK9 administration, which was blocked by 316P but not by the non-hPCSK9 specific mAb (FIG. 10).

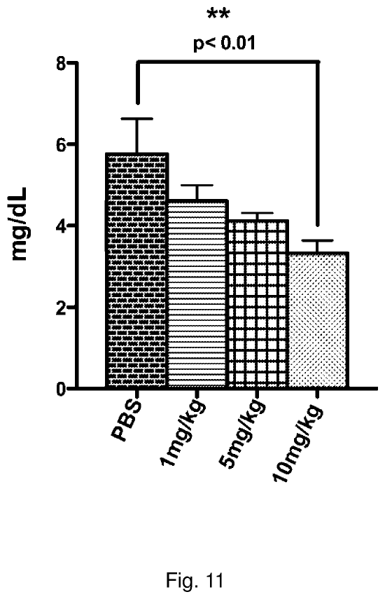

Effect of different doses of 316P was also evaluated in PCSK9^{hu/hu} mice with both elevated LDL-C and elevated hPCSK9 levels. PCSK9^{hu/hu} mice were first placed on a high carbohydrate diet for 8 weeks, resulting in a ˜2-fold elevation in both LDL-C and hPCSK9 levels. Either 316P or a non-hPCSK9 specific mAb, each at 1 mg/kg, 5 mg/kg, or 10 mg/kg, were administered to the mice. Sera were collected 24 hours later and LDL-C levels were analyzed. 316P was effective in decreasing LDL-C levels in a dose-dependent manner (FIG. 11). In addition, 316P administered at a dose of 10 mg/kg, rapidly reduced LDL-C levels back to original (pre-diet) values within 24 hours.

### Example 15. Mouse PK Studies

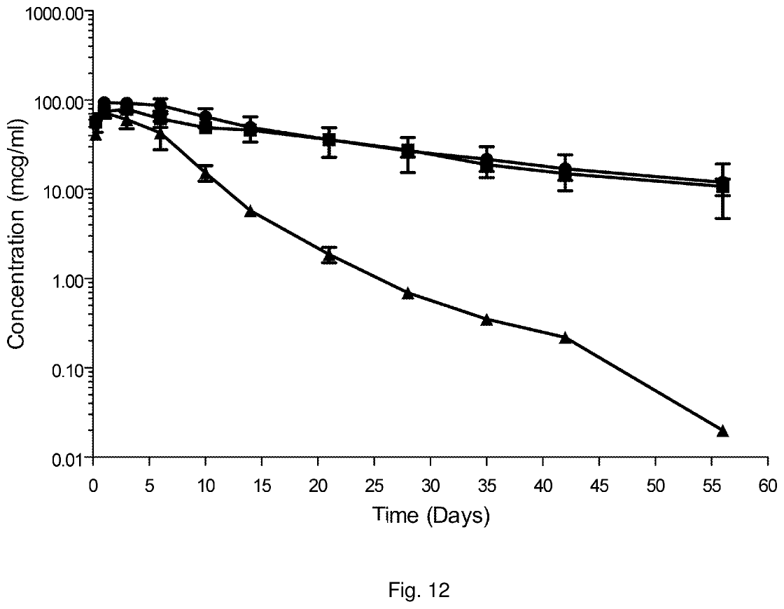

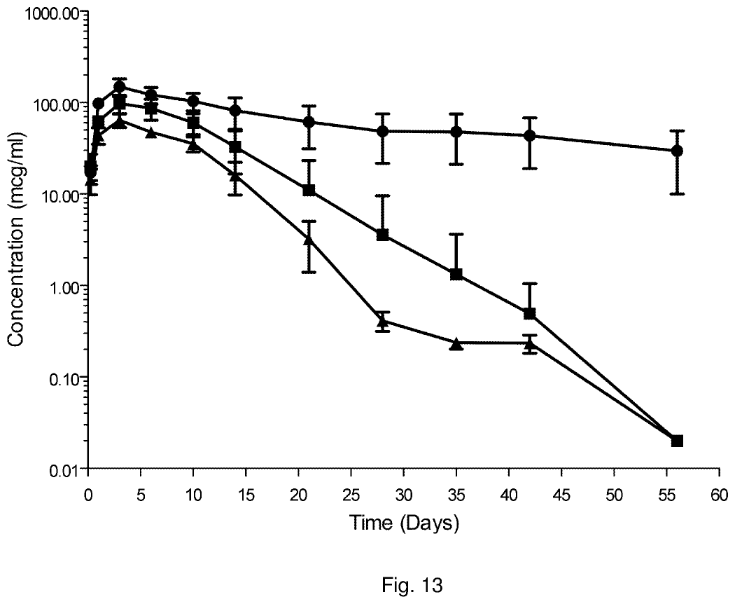

A PK study was conducted in 6-week-old C57BL/6 mice and 11-15 week old hPCSK9 heterozygous mice. A single injection of Control I, 316P, or 300N, each at 10 mg/kg, was administered SC. Serum bleeds were measured for hIgG levels at 0 hr (pre-bleed), 6 hr, day 1, 3, 6, 10, 14, 21, 28, 35, 42 and 56, for a total of 12 time points, using an anti-hFc capture and anti-hFc detection sandwich ELISA (FIGS. 12 and 13). All mAbs achieved their Tₘₐₓ at approximately 3 days with corresponding Cₘₐₓ levels of approximately 47-115 μg/ml for C57BL/6 mice and 55-196 μg/ml for hPCSK9 heterozygous mice. At Day 56, Control I mAb levels were about 12 μg/ml and 300N levels were about 11 μg/ml whereas 316P levels were about less than 0.02 μg/ml in C57BL/6 mice. At Day 56 in hPCSK9 heterozygous mice, Control I mAb levels were about 29 μg/ml, while both 300N and 316P levels were below the quantifiable limit (BQL) of 0.02 μg/ml.

### Example 16. Anti-hPCSK9 Antibody Binding to Mutant/Variant hPCSK9

To further assess binding between hPCSK9 and anti-hPCSK9 mAbs, 21 variant hPCSK9 proteins in which each variant contained a single point mutation and two variant hPCSK9 proteins each contained a double mutation were generated. Each selected antibody was captured on a F(ab′)₂ anti-hIgG surface created through direct chemical coupling to a BIACORE™ chip to form a captured antibody surface. Each mmh-tagged variant hPCSK9 at varying concentrations from 100 nM to 25 nM was then injected over the captured antibody surface at a flowrate of 60 μl/min for 240 sec, and the dissociation of variant hPCSK9 and antibody was monitored in real time for 20 min at 25° C. nb: no binding was observed under these experimental conditions (K_{D}=M×10⁻⁹; T_{1/2}=min; WT=wildtype).

TABLE 27

| | 316P | | 300N | | Control I | | Control II | | Control III | |
|---|---|---|---|---|---|---|---|---|---|---|
| | K_{D} | T_{1/2} | K_{D} | T_{1/2} | K_{D} | T_{1/2} | K_{D} | T_{1/2} | K_{D} | T_{1/2} |
| WT | 1.00 | 37 | 0.69 | 120 | 30.6 | 16 | 0.10 | 333 | 0.60 | 481 |
| P70A | 1.42 | 32 | 1.68 | 80 | 19.0 | 16 | 0.24 | 168 | 0.90 | 325 |
| S127R | 2.40 | 36 | 1.87 | 110 | 25.0 | 18 | 0.26 | 288 | 0.55 | 550 |
| D129G | 1.27 | 36 | 1.40 | 88 | 22.9 | 18 | 0.19 | 257 | 0.75 | 445 |
| S147F | 1.29 | 32 | 9.07 | 24 | 21.1 | 15 | 0.22 | 178 | 0.23 | 1468 |
| S153R | 5.64 | 4 | 0.56 | 141 | 36.6 | 17 | 0.09 | 322 | 3.33 | 60 |
| E159R | 6.96 | 5 | 0.82 | 94 | 31.7 | 16 | 0.08 | 350 | 2.97 | 68 |
| T162R | 0.98 | 43 | 0.58 | 140 | 29.0 | 17 | 0.09 | 322 | 0.48 | 362 |
| D192R | 1.35 | 28 | 0.75 | 119 | 30.2 | 15 | 0.09 | 326 | nb | nb |
| R194E | 0.38 | 71 | 0.65 | 129 | 31.4 | 16 | 0.07 | 389 | nb | nb |
| E197R | 1.42 | 27 | 0.67 | 115 | 30.2 | 17 | 0.09 | 339 | nb | nb |
| R215H | 0.86 | 41 | 1.03 | 98 | 37.8 | 17 | 0.65 | 49 | 0.74 | 272 |
| R215E | 0.90 | 43 | 1.81 | 77 | 44.0 | 16 | 4.48 | 12 | 0.78 | 276 |
| F216L | 1.83 | 32 | 0.99 | 121 | 21.2 | 15 | 1.35 | 39 | 0.33 | 880 |
| R237E | 2.48 | 15 | 1.03 | 109 | 29.6 | 15 | 0.07 | 481 | 5.89 | 43 |
| D238R | 410 | 1 | 0.78 | 123 | 25.9 | 19 | 0.24 | 144 | 0.14 | 1273 |
| A341R | 1.54 | 21 | 0.34 | 190 | 28.7 | 18 | 0.08 | 340 | 0.88 | 200 |
| D343R | 7.88 | 6 | 1.18 | 89 | 27.0 | 16 | 0.08 | 402 | 4.13 | 66 |
| R357H | 6.26 | 30 | 6.53 | 66 | 26.4 | 13 | 0.63 | 165 | 1.91 | 896 |
| E366K | 2.92 | 13 | 36.0 | 2 | 28.8 | 18 | 0.46 | 69 | 0.38 | 808 |
| D374Y | 2.04 | 15 | 0.66 | 83 | 25.0 | 17 | 0.08 | 285 | 1.02 | 161 |
| V380M | 0.48 | 63 | 2.82 | 28 | 25.9 | 17 | 0.15 | 177 | 0.35 | 711 |
| P70A, | 1.18 | 34 | 7.87 | 24 | 23.5 | 18 | 0.23 | 164 | 0.79 | 348 |
| S147F | | | | | | | | | | |
| E366K, | 3.33 | 12 | 78.3 | 1 | 25.5 | 18 | 0.59 | 60 | 0.52 | 551 |
| V380M | | | | | | | | | | |

The results show that when residue D238 was mutated, the binding affinity of 316P for hPCSK9 was reduced >400-fold, from a K_{D} of 1×10⁻⁹ M to 410×10⁻⁹ M; and T_{1/2} shortened about 30-fold, from 37 to 1 min, indicating that 316P binds an epitope on hPCSK9 comprising D238 of hPCSK9 (SEQ ID NO:755). Additionally, BIACORE™ assays show that 316P binding affinity and T_{1/2} were reduced about 5-to 10-fold when a residue at 153, 159 or 343 was mutated. Specifically, K_{D} was reduced from about 1×10⁻⁹ M to between about 5-8×10⁻⁹ M when any one of S153, E159 or D343 were mutated; while T_{1/2} was decreased from about 37 min to between about 4-6 min.

300N binding to hPCSK9 was reduced about 50-fold when the residue at position 366 was mutated, resulting in a decreased K_{D} of from about 0.7×10⁻⁹ M to about 36×10⁻⁹ M and a shorter T_{1/2} from about 120 to 2 min. These results indicate that 300N binds an epitope on hPCSK9 comprising E366 of hPCSK9 (SEQ ID NO:755). Additionally, the BIACORE™ assays show that 300N binding affinity and T_{1/2} were reduced between 2- to >10-fold when a residue at 147 or 380 was mutated. Specifically, K_{D} was reduced from about 0.69×10⁻⁹ M to between about 2-9×10⁻⁹ M when any of S147 or V380 were mutated; while T_{1/2} was shortened from about 120 min to between about 24-66 min. Compared to 316P, 300N binding to hPCSK9 was not reduced by a mutation at residue 238.

In contrast, Control I antibody did not exhibit an altered binding affinity or T_{1/2} in response to any of the positional mutations tested; Control IU antibody exhibited a 40-fold decreased affinity when residue 215 was mutated (R215E) (from ˜0.1×10⁻⁹ to ˜4.5×10⁻⁹), and T_{1/2} was about 27-fold shorter (from ˜333 to 12 min); while Control III antibody exhibited a decreased affinity when residue 237 was mutated (K_{D} decreased from ˜0.6×10⁻⁹ to ˜5.9×10⁻⁹, and T_{1/2} decreased from ˜481 to ˜43 min).

Binding specificity of 316P, 300N, and control anti-hPCSK9 mAbs to hPCSK9 variants was tested using an ELISA-based immunoassay. Anti-PCSK9 mAbs were coated on a 96-well plate overnight at 4° C. Each mmh-tagged variant hPCSK9 in CHO-k1 transient transfection lysate supernatants was added to the antibody-coated plate at various concentrations ranging from 0 to 5 nM. After 1 hr binding at RT, the plate was washed and bound variant hPCSK9 was detected using HRP-conjugated anti-myc polyclonal antibody (−=OD <0.7; +=OD 0.7-1.5; ++=OD>1.5).

TABLE 28

| | | | Con- | Con- | Con- |
|---|---|---|---|---|---|
| | | | trol | trol | trol |
| hPCSK9 or Variant | 316P | 300N | I | II | III |
| hPCSK9(WT) | ++ | ++ | ++ | ++ | ++ |
| hPCSK9(S127R) | ++ | ++ | ++ | ++ | ++ |
| hPCSK9(D129G) | ++ | ++ | ++ | ++ | ++ |
| hPCSK9(S153R) | ++ | ++ | ++ | ++ | ++ |
| hPCSK9(R215H) | ++ | ++ | ++ | ++ | ++ |
| hPCSK9(F216L) | ++ | ++ | ++ | ++ | ++ |
| hPCSK9(R237E) | ++ | ++ | ++ | ++ | ++ |
| hPCSK9(D238R) | − | ++ | ++ | ++ | ++ |
| hPCSK9(A341R) | ++ | ++ | ++ | ++ | ++ |
| hPCSK9(D343R) | ++ | ++ | ++ | ++ | ++ |
| hPCSK9(R357H) | ++ | ++ | ++ | ++ | ++ |
| hPCSK9(E159R) | ++ | ++ | ++ | ++ | ++ |
| hPCSK9(T162R) | ++ | ++ | ++ | ++ | ++ |
| HPCSK9(D192R) | ++ | ++ | ++ | ++ | − |
| hPCSK9(R194E) | ++ | ++ | ++ | ++ | − |
| hPCSK9(E197R) | ++ | ++ | ++ | ++ | − |
| hPCSK9(R215E) | ++ | ++ | ++ | ++ | ++ |
| hPCSK9(P70A) | ++ | ++ | ++ | ++ | ++ |
| hPCSK9(S147F) | ++ | ++ | ++ | ++ | ++ |
| hPCSK9(E366K) | ++ | + | ++ | ++ | ++ |
| hPCSK9(V380M) | ++ | ++ | ++ | ++ | ++ |
| hPCSK9(P70A, S147F) | ++ | ++ | ++ | ++ | ++ |
| hPCSK9(E366K, | ++ | + | ++ | ++ | ++ |
| V380M) | | | | | |

### Example 17. Effect of 316P on Normolipemic and Hyperlipemic Hamster

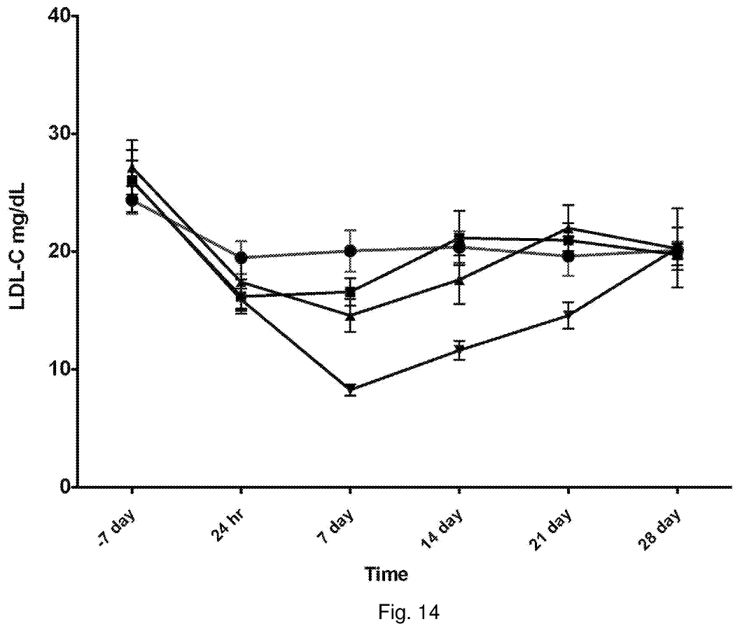

The ability of anti-PCSK9 mAb 316P to reduce serum LDL-C was tested in normolipemic or hyperlipemic Gold Syrian hamsters (Mesocricetus auratus). Male Syrian Hamsters, age 6-8 weeks, weighing between 80-100 grams, were allowed to acclimate for a period of 7 days before entry into the study. All animals were placed on either a standard chow diet or a hyperlipemic diet of chow supplemented with 0.1% cholesterol and 10% coconut oil. The 316P mAb was delivered to hamsters by a single subcutaneous injection at doses of 1, 3, or 10 mg/kg for normolipemic hamsters and at doses of 3, 10, or 30 mg/kg for hyperlipemic hamsters. Serum samples were taken from all groups at 24 hr and 7, 14, and 22 days post injection, at which time serum lipid levels were assessed and compared to baseline levels taken 7 days prior to the administration of the mAbs. Circulating total cholesterol and LDL-C in normolipemic hamsters was significantly reduced in a dose-dependent manner compared to vehicle injection. As shown in FIG. 14, administration of 316P effectively reduced LDL-C levels by up to 60% seven days post injection at the highest dose (10 mg/kg) tested. Similar cholesterol reducing effect of 316P was not observed in hyperlipemic hamsters.

We claim:

1. A human antibody or antigen-binding fragment of a human antibody that specifically binds human proprotein convertase subtilisin/kexin type 9 (hPCSK9), characterized by one or more of the following:
(i) capable of reducing serum total cholesterol at least about 25 to 35% and sustaining the reduction over at least a 24 day period relative to a predose level;
(ii) capable of reducing serum LDL cholesterol at least about 65-80% and sustaining the reduction over at least a 24 day period relative to a predose level or capable of reducing serum LDL cholesterol at least about 40-70% and sustaining the reduction over at least a 60 to 90 day period relative to a predose level;
(iii) capable of reducing serum triglyceride at least about 25-40% relative to predose level;
(iv) achieves one or more of (i)-(iii) without reducing serum HDL cholesterol or reducing serum HDL cholesterol no more than 5% relative to predose level;
(v) achieves one or more of (i)-(iii) with little or no measurable effect on liver function, as determined by ALT and AST measurements.

2. The antibody or antigen-binding fragment according to claim 1, comprising
a heavy chain CDR3 (HCDR3) domain selected from the group consisting of SEQ ID NO:8, 32, 56, 80, 104, 128, 152, 176, 200, 224, 248, 272, 296, 320, 344, 368, 392, 416, 440, 464, 488, 512, 536, 560, 584, 608, 632, 656, 680, 704 and 728; and
a light chain CDR3 (LCDR3) domain selected from the group consisting of SEQ ID NO:16, 40, 64, 88, 112, 136, 160, 184, 208, 232, 256, 280, 304, 328, 352, 376, 400, 424, 448, 472, 496, 520, 544, 568, 592, 616, 639, 664, 688, 712 and 736.

3. The antibody or antigen-binding fragment according to claim 2, wherein the HCDR3/LCDR3 is SEQ ID NO:80/88 or 224/232.

4. The antibody or antigen-binding fragment according to claim 2, further comprising
a heavy chain CDR1 (HCDR1) domain selected from the group consisting of SEQ ID NO:4, 28, 52, 76, 100, 124, 148, 172, 196, 220, 244, 268, 292, 316, 340, 364, 388, 412, 436, 460, 484, 508, 532, 556, 580, 604, 628, 652, 676, 7000 and 724;
a heavy chain CDR2 (HCDR2) domain selected from the group consisting of SEQ ID NO:6, 30, 54, 78, 102, 126, 150, 174, 198, 222, 246, 270, 294, 318, 342, 366, 390, 414, 438, 462, 486, 510, 534, 558, 582, 606, 630, 654, 678, 702 and 726;
a light chain CDR1 (LCDR1) domain selected from the group consisting of SEQ ID NO:12, 36, 60, 84, 108, 132, 156, 180, 204, 228, 252, 276, 300, 324, 348, 372, 396, 420, 444, 468, 492, 516, 540, 564, 588, 612, 636, 660, 684, 708 and 732; and
a light chain CDR2 (LCDR2) domain selected from the group consisting of SEQ ID NO:14, 38, 62, 86, 110, 134, 158, 182, 206, 230, 254, 278, 302, 326, 350, 374, 398, 422, 446, 470, 494, 518, 542, 566, 590, 614, 638, 662, 686, 710 and 734.

5. The antibody or antigen-binding fragment according to claim 4, wherein the light and heavy chain CDR sequences are selected from the group consisting of SEQ ID NO:76, 78, 80, 84, 86, 88;
SEQ ID NO:220, 222, 224, 228, 230, 232.

6. The antibody or antigen-binding fragment according to any one of the above claims, wherein the HCVR is SEQ ID NO:90 or 218, and the LCVR is SEQ ID NO:90 or 218.

7. An antibody or antigen-binding fragment according to claim 1, comprising a HCDR3 and a LCDR3 sequence, wherein
the HCDR3 comprises an amino acid sequence of the formula X¹-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸-X¹⁹-X²⁰ (SEQ ID NO:747) wherein X¹ is Ala, X² is Arg or Lys, X³ is Asp, X⁴ is Ser or Ile, X⁵ is Asn or Val, X⁶ is Leu or Trp, X⁷ is Gly or Met, X⁸ is Asn or Val, X⁹ is Phe or Tyr, X¹⁰ is Asp, X¹¹ is Leu or Met, X¹² is Asp or absent, X¹³ is Tyr or absent, X¹⁴ is Tyr or absent, X¹⁵ is Tyr or absent, X¹⁶ is Tyr or absent, X¹⁷ is Gly or absent, X¹⁸ is Met or absent, X¹⁹ is Asp or absent, and X²⁰ is Val or absent; and
the LCDR3 comprises an amino acid sequence of the formula X¹-X²-X³-X⁴-X⁵-X⁶-X⁷-X⁸-X⁹ (SEQ ID NO:750) wherein X¹ is Gln or Met, X² is Gln, X³ is Tyr or Thr, X⁴ is Tyr or Leu, X⁵ is Thr or Gln, X⁶ is Thr, X⁷ is Pro, X⁸ is Tyr or Leu, and X⁹ is Thr.

8. The antibody or antigen-binding fragment according to claim 7, further comprising a HCDR1, HCDR2, LCDR1 and LCDR2, wherein
the HCDR1 comprises an amino acid sequence of the formula X¹-X²-X³-X⁴-X⁵-X⁶-X⁷-X⁸ (SEQ ID NO:745), wherein X¹ is Gly, X² is Phe, X³ is Thr, X⁴ is Phe, X⁵ is Ser or Asn, X⁶ is Ser or Asn, X⁷ is Tyr or His, and X⁸ is Ala or Trp;
the HCDR2 comprises an amino acid sequence of the formula X¹-X²-X³-X⁴-X⁵-X⁶-X⁷ X⁸ (SEQ ID NO:746), wherein X¹ is Ile, X² is Ser or Asn, X³ is Gly or Gln, X⁴ is Asp or Ser, X⁵ is Gly, X⁶ is Ser or Gly, X⁷ is Thr or Glu, and X⁸ is Thr or Lys;
the LCDR1 comprises an amino acid sequence of the formula X¹-X²-X³-X⁴-X⁵-X⁶-X⁷-X⁸-X⁹-X¹⁰-X¹¹-X¹² (SEQ ID NO:748) wherein X¹ is Gln, X² is Ser, X³ is Val or Leu, X⁴ is Leu, X⁵is His or Tyr, X⁶ is Arg or Ser, X⁷ is Ser or Asn, X⁸ is Asn or Gly, X⁹ is Asn, X¹⁰ is Arg or Asn, X¹¹ is Asn or Tyr, and X¹² is Phe or absent;
the LCDR2 comprises an amino acid sequence of the formula X¹-X²-X³ (SEQ ID NO:749) wherein X¹ is Trp or Leu, X² is Ala or Gly, and X³ is Ser.

9. The antibody or antigen-binding fragment according to any one of the above claims, wherein the binding of the antibody or fragment thereof to a variant PCSK9 protein is less than 50% of the binding between the antibody or fragment thereof and the PCSK9 protein of SEQ ID NO:755.

10. The antibody or antigen-binding fragment according to claim 9, wherein the variant PCSK9 protein comprises at least one mutation of a residue at a position selected from the group of
S153, E159, D238 and D343; or
S147, E366 and V380.

11. The antibody or antigen-binding fragment according to any one of the above claims, comprising a heavy chain variable region (HCVR) encoded by nucleotide sequence segments derived from V_{H}, D_{H} and J_{H} germline gene segments, and a light chain variable region (LCVR) encoded by nucleotide sequence segments derived from V and J_{K} germline gene segments, wherein the germline sequences are:
a) V_{H} gene 3-23, D_{H} gene 7-27, J_{H} gene 2, V_{K} gene 4-1, J_{K} gene 2, or
b) V_{H} gene 3-7, D_{H} gene 2-8, J_{H} gene 6, V_{K} gene 2-28, J_{K} gene 4.

12. An isolated nucleic acid molecule encoding the antibody or antigen-binding fragment according to any one of the above claims.

13. An expression vector comprising the nucleic acid molecule according to claim 11.

14. A method of producing an anti-human PCSK9 antibody or antigen-binding fragment of an antibody comprising the steps of introducing the expression vector according to claim 13 into an isolated host cell, growing the cell under conditions permitting production of the antibody or fragment thereof, and recovering the antibody or fragment so produced.

15. A pharmaceutical composition comprising the antibody or antigen-binding fragment according to claim 1 to claim 11, and a pharmaceutically acceptable carrier.

16. The pharmaceutical composition according to claim 15, further comprising a second therapeutic agent, wherein the second therapeutic agent is selected from an inhibitor of 3-hydroxy-3-methylglutaryl (HMG)-coenzyme A (CoA) reductase, a statin, an inhibitor of cholesterol uptake and or bile acid re-absorption, an agent which increases lipoprotein catabolismor an activator of LXR transcription factor.

17. A method for treating a disease or condition which is ameliorated, improved, inhibited or prevented with a PCSK9 antagonist, comprising administering a therapeutic amount of the pharmaceutical composition according to claim 15 or 16 to a subject in need thereof.

18. The method according to claim 17, wherein the subject is a human subject suffering from hypercholesterolemia, hyperlipidemia, indicated for LDL apheresis, identified as heterozygous for Familial Hypercholesterolemia, statin intolerant. statin uncontrolled, at risk for developing hypercholesterolemia, dyslipidemia, cholestatic liver disease, nephrotic syndrome, hypothyroidism, obesity, atherosclerosis and cardiovascular diseases.

19. An antibody or antigen-binding fragment of an antibody according to any one of claims 1 to 11 for use to attenuate or inhibit a PCSK9-mediated disease or condition.

20. Use of an antibody or antigen-binding fragment of an antibody according to any one of claims 1 to 11 in the manufacture of a medicament for use to attenuate or inhibit a PCSK9-mediated disease or condition.

21. Use according to claim 20, wherein the PCSK9-mediated disease or condition is hypercholesterolemia, hyperlipidemia, LDL apheresis, heterozygous for Familial Hypercholesterolemia, statin intolerant. statin uncontrolled, risk for developing hypercholesterolemia, dyslipidemia, cholestatic liver disease, nephrotic syndrome, hypothyroidism, obesity, atherosclerosis and cardiovascular diseases.
